(19) United States
(12) Patent Application Publication
(10) Pub. No.: US 2016/0251341 A1
(43) Pub. Date: Sep. 1, 2016

(54) SUBSTITUTED TRIAZOLE COMPOUNDS AS SERINE PROTEASE INHIBITORS

(71) Applicant: Verseon Corporation, Fremont, CA (US)

(72) Inventor: Kevin Michael Short, Fremont, CA (US)

(21) Appl. No.: 15/056,901

(22) Filed: Feb. 29, 2016

Related U.S. Application Data
(60) Provisional application No. 62/126,432, filed on Feb. 27, 2015.

(51) Int. Cl. C07D 409/14; C07D 403/04; C07D 249/14; A61K 9/00; C07D 249/08

(52) U.S. Cl. CPC C07D 409/14; A61K 9/0048; C07D 249/08; C07D 249/14; C07D 403/04

(57) ABSTRACT

There are provided inter alia multisubstituted aromatic compounds useful for the inhibition of thrombin and/or kallikrein, which compounds include substituted triazolyl. There are additionally provided pharmaceutical compositions. There are additionally provided methods of treating and preventing certain diseases or disorders, which diseases or disorders are amenable to treatment or prevention by the inhibition of thrombin and/or kallikrein.

### CROSS-REFERENCE TO RELATED APPLICATIONS

The present application claims the benefit of priority under 35 U.S.C. §119(e) to U.S. Provisional Application No. 62/126,432, SUBSTITUTED TRIAZOLE COMPOUNDS AS SERINE PROTEASE INHIBITORS, filed on Feb. 27, 2015, which is currently co-pending herewith and which is incorporated by reference in its entirety and for all purposes.

### BACKGROUND OF THE INVENTION

The present disclosure relates to compounds, e.g., certain substituted triazole compounds, which exhibit biological activity, e.g., inhibitory action, against serine proteases, including thrombin and plasma kallikrein.

Serine proteases are a large family of enzymes with diverse biological functions, their commonality being the presence and critical function of the active-site serine residue. Their central function is the catalytic scission of peptide bond substrates via a Ser, His, Asp triad within the active site (Kraut, J. Annual Review of Biochemistry 1977, 46, 331-358). The present disclosure relates to compounds, e.g., heterocycloalkyl-substituted triazolyl compounds, which exhibit biological activity, e.g., inhibitory action, against serine proteases, including thrombin and various kallikreins.

In mammalian systems, blood vessel injuries result in bleeding events, which are dealt with by the blood coagulation cascade. The cascade includes the Extrinsic and Intrinsic pathways, involving the activation of at least 13 interconnected factors and a variety of co-factors and other regulatory proteins. Upon vascular injury, plasma factor VII interacts with exposed Tissue Factor (TF), and the resultant TF-fVIIa complex initiates a complex series of events. Factor fXa is produced directly ‘downstream’ from the TF-fVIIa complex, and amplified manifold via the Intrinsic Pathway. FXa then serves as the catalyst for formation of thrombin (ffIa), which in turn is the direct precursor to fibrinolysis. The outcome is a fibrinolytic clot, which stops the bleeding. Fibrinolysis of the polymeric clot into fibrin monomers leads to dissolution and a return of the system to the pre-clot state. The cascade is a complex balance of factors and co-factors and is tightly regulated.

In disease states, undesired up- or down-regulation of any factor leads to conditions such as bleeding or thrombosis. Historically, anticoagulants have been used in patients at risk of suffering from thrombotic complications, such as angina, stroke and heart attack. Warfarin has enjoyed dominance as a first-in-line anticoagulant therapeutic. Developed in the 1940s, it is a Vitamin K antagonist and inhibits factors II, VII, IX and X, amongst others. It is administered orally, but its ease of use is tempered by other effects: it has a very long half life (>2 days) and has serious drug-drug interactions. Importantly, since Vitamin K is a ubiquitous cofactor within the coagulation cascade, antagonism results in the simultaneous inhibition of many clotting factors and thus can lead to significant bleeding complications.

Much attention has been focused on heparin, the naturally-occurring polysaccharide that activates AT III, the endogenous inhibitor of many of the factors in the coagulation cascade. The need for parenteral administration for the heparin-derived therapeutics, and the inconvenient requirements for close supervision for the orally available warfarin, has resulted in a drive to discover and develop orally available drugs with wide therapeutic windows for safety and efficacy.

Indeed, the position of thrombin in the coagulation cascade has made it a popular target for drug discovery. Thrombin is a central protein in the coagulation process, which is activated and amplified upon vascular injury. Thrombin generation prompts a cascade in various factors in the coagulation cascade, ultimately depositing fibrin, the framework for a clot. The clot causes cessation of the bleeding event accompanying the vascular injury. Thrombin and associated protein ultimately cause dissolution of the clot through ‘fibrinolysis’, returning the system back to the pre-injury state. In a ‘normal’ state of injury, this thrombin generation and clot deposition is desired. In a disease state, clot deposition is undesired. General thrombotic events are the clinical result of clot deposition and accumulation in the arteries, veins or within the heart. Eventual break-off of the accumulated clot structure into the vascular system causes the clot to travel to the brain and/or lungs, resulting in a stroke, myocardial infarction (heart attack), pulmonary embolism, paralysis and consequent death. Compounds that inhibit thrombin have been shown in the literature to be useful as anticoagulants in vitro and in vivo, and in the clinic in patients have been shown to fulfil a critically unmet medical need. A thorough discussion of thrombin and its roles in the coagulation process can be found in a variety of references, including the following which are incorporated herein by reference in their entireties and for all purposes: Wieland, H. A., et al., 2003, Curr Opin Investig Drugs, 4:264-71; Gross, P. L. & Weitz, J. I., 2008, Arterioscler Thromb Vasc Biol, 28:380-6; Hirsh, J., et al., 2005, Blood, 105:453-63; Prezelj, A., et al., 2007, Curr Pharm Des, 13:287-312. Without further wishing to be bound by any theory, it is believed that the use of direct thrombin inhibitors (DTIs) is very well precedented, such as with the hirudin-based anticoagulants, and thus there is strong interest in the discovery and development of novel DTIs, particularly those with selectivity for inhibiting thrombin over other related serine proteases. Kallikreins are a subgroup of serine proteases, divided into plasma kallikrein and tissue kallikreins. Plasma kallikrein (KLKB1) liberates kinins (bradykinin and kallidin) from the kininogens, peptides responsible for the regulation of blood pressure and activation of inflammation. In the contact activation pathway of the coagulation cascade, plasma kallikrein assists in the conversion of factor XII to factor XIIa (Keel, M.; Trentz, O. Injury 2005, 36, 691-709). Factor XIIa converts factor XI into factor XIa, which in turn activates factor IX, which with its co-factor factor VIIIa forms the tenase complex, which finally activates factor X to factor Xa. In the fibrinolysis part of the coagulation cascade, plasma kallikrein serves to convert plasminogen to plasmin. Thus, it has been proposed that plasma kallikrein inhibitors can be useful in the treatment of thrombotic and fibrinolytic diseases and disease conditions (U.S. Pat. No. 7,625,944; Bird et al. Thrombosis and Hemostasis 2012, 107, Dhaval Kolte, M D. et al., Cardiology in Review, 2015).

Tissue kallikreins (KLKs, for example, KLK1) are subdivided into various types, and have been extensively investigated in cancer and inflammation biology. Various kallikrein KLKs have been found to be up- or down-regulated in various cancer types, such as cervical-, testicular-, and non-small-cell lung adenocarcinoma (Caliendo et al. J. Med. Chem., 2012, 55, 6669). Furthermore, overexpression of various KLKs in the skin has led to the recognition that certain kallikrein inhibitors can be useful for certain dermatological conditions, including atopic dermatitis, psoriasis and rare skin diseases such as Netherton Syndrome (Freitas et al. Bioorganic & Medicinal Chemistry Letters 2012, 22, 6072-6075). A thorough discussion of tissue kallikreins, plasma kallikrein, their functions and potential roles in various diseases can be found in a variety of references, including the following which are incorporated herein by reference in their entireties and for all purposes: Renné, T.; Gruber, A. Thromb Haemost 2012, 107, 1012-3; Sotiropoulou, G.; Pampalakis, G. Trends in Pharmacological Sciences 2012, 33, 623-634; Pampalakis, G.; Sotiropoulou, G. Chapter 9 Pharmacological Targeting of Human Tissue Kallikrein-Related Peptidases. In Proteinases as Drug Targets, Dunn, B., Ed. The Royal Society of Chemistry: 2012; pp 199-228; Caliendo, G.; Santagada, V.; Perissutti, E.; Severino, B.; Fiorino, F.; Frecentese, F.; Juliano, L. J Med Chem 2012, 55, 6669-86.

### BRIEF SUMMARY OF THE INVENTION

Embodiments of the invention encompass compounds with the following structure:

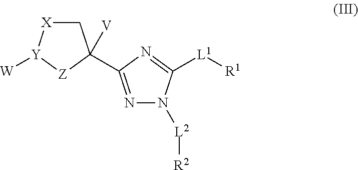

or pharmaceutically acceptable salt, ester, solvate, or prodrug thereof, wherein:
L¹ and L² are independently a bond, substituted or unsubstituted alkylene, substituted or unsubstituted heteroalkylene, —C(O)—, —S—, —SO—, —SO₂—, —O—, —NHSO₂—, —NHC(O)—, or —NR⁴—;
R¹ and R² are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted fused ring aryl, or substituted or unsubstituted heteroaryl;
R⁴ is hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted fused ring aryl, or substituted or unsubstituted heteroaryl;
V is hydrogen or substituted or unsubstituted alkyl;
W is absent, hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted heteroaryl, —C(O)R⁶, —C(O)OR⁶, —C(O)NR⁶R⁷, —SR⁶, —SOR⁶, —SO₂R⁶, —SO₂NR⁶R⁷, —OR⁶, —NHSO₂R⁶, or —NR⁶R⁷, where R⁶ and R⁷ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, or substituted or unsubstituted heteroaryl, wherein R⁶ and R⁷ can be combined if both are present to form a substituted or unsubstituted alkylene, or substituted or unsubstituted heteroalkylene;
X is a bond, substituted or unsubstituted alkylene, —O—, or —NR⁸—;
Y is a bond, substituted or unsubstituted alkylene, —O—, or —N—, provided that when Y is —O—, W is absent; and
Z is a bond, —C(O)—, substituted or unsubstituted alkylene, —O—, or —NR⁹—;
wherein R⁸ and R⁹ are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted heteroaryl, —C(O)R⁶, —C(O)OR⁶, —C(O)NR⁶R⁷, —SR⁶, —SOR⁶, —SO₂R⁶, —SO₂NR⁶R⁷, —OR⁶, —NHSO₂R⁶, or —NR⁶R⁷, wherein R⁶ and R⁷ are as defined above; and
provided that either at least one of X is —O— or —NR⁸—, Y is —O— or —N—, or Z is —O— or —NR⁹—.

In some embodiments, the compound can be a pharmaceutically acceptable salt, ester, solvate, or prodrug of a compound of Formula (III). In some embodiments, the compound is not an ester, not a solvate, and not a prodrug.

In some embodiments, X can be a bond or substituted or unsubstituted alkylene. In some embodiments, Z can be a bond or substituted or unsubstituted alkylene. In some embodiments, X can be a bond or substituted or unsubstituted alkylene, and Z can be a bond or substituted or unsubstituted alkylene.

Some embodiments include compounds where X is a bond or substituted or unsubstituted alkylene and Z is a bond or substituted or unsubstituted alkylene, Y can be —N—, and W can be hydrogen, substituted or unsubstituted alkyl, substituted or unsubstituted heteroalkyl, substituted or unsubstituted aryl, —C(O)R⁶, —C(O)OR⁶, —C(O)NR⁶R⁷′—SO₂R⁶, or SO₂NR⁶R⁷, wherein R⁶ and R⁷ can be independently substituted or unsubstituted alkyl, substituted or unsubstituted cycloalkyl, or substituted or unsubstituted heterocycloalkyl, or R⁶ and R⁷ can be combined if both are present to form a substituted or unsubstituted alkylene, or substituted or unsubstituted heteroalkylene.

Some embodiments include compounds where X can be substituted or unsubstituted methylene, substituted or unsubstituted ethylene, substituted or unsubstituted propylene, substituted or unsubstituted butylene, or substituted or unsubstituted pentylene, and Z can be a bond. In some embodiments, X can be a bond, and Z can be substituted or unsubstituted methylene, substituted or unsubstituted ethylene, substituted or unsubstituted propylene, substituted or unsubstituted butylene, or substituted or unsubstituted pentylene. In some embodiments, X can be substituted or unsubstituted methylene, substituted or unsubstituted ethylene, substituted or unsubstituted propylene, substituted or unsubstituted butylene, or substituted or unsubstituted pentylene, and Z can be substituted or unsubstituted methylene, substituted or unsubstituted ethylene, substituted or unsubstituted propylene, substituted or unsubstituted butylene, or substituted or unsubstituted pentylene. In some embodiments, X and Z can both be branched alkylene, and X and Z can be covalently attached. In some embodiments, Z can be substituted methylene, substituted ethylene, substituted propylene, substituted butylene, or substituted pentylene, having one or more substituent groups which can be —OH, —NH₂, —SH, —CN, —CF₃, —NO₂, oxo, halogen, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Some embodiments include compounds where X can be substituted or unsubstituted methylene, substituted or unsubstituted ethylene, substituted or unsubstituted propylene, substituted or unsubstituted butylene, or substituted or unsubstituted pentylene, and Z can be —C(O)—. In some embodiments, W can be hydrogen. In some embodiments, W can be substituted or unsubstituted alkyl, substituted or unsubstituted heteroalkyl, substituted or unsubstituted alkenyl, substituted or unsubstituted heteroalkenyl, substituted or unsubstituted cycloalkyl, substituted or unsubstituted heterocycloalkyl, —C(O)R⁶, —C(O)OR⁶, —C(O)NR⁶R⁷′—SR⁶, —SOR⁶, —SO₂R⁶, or —SO₂NR⁶. In some embodiments, W can be substituted alkyl, substituted heteroalkyl, substituted alkenyl, substituted heteroalkenyl, substituted cycloalkyl, or substituted heterocycloalkyl, having one or more substituent which can be —OH, —NH₂, —SH, —CN, —CF₃, —NO₂, oxo, halogen, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, W can be —COR⁶, —C(O)OR⁶, —C(O)NR⁶R⁷, —SO₂R⁶ or —SO₂NR⁶R⁷, where R⁶ and R⁷ can be selected from the group consisting of substituted or unsubstituted alkyl, substituted or unsubstituted heteroalkyl, substituted or unsubstituted aryl, or substituted or unsubstituted heteroaryl, or R⁶ and R⁷ can be combined to form a substituted or unsubstituted alkylene.

Some embodiments include compounds where W can be absent, X can be —NR⁸—, Y can be a bond or substituted or unsubstituted alkylene, and Z can be —NR⁹—. In some embodiments wherein W can be absent, X can be —NR⁸—, Y can be a bond or substituted or unsubstituted alkylene, and Z can be —NR⁹—, Y can be substituted or unsubstituted methylene, substituted or unsubstituted ethylene, substituted or unsubstituted propylene, substituted or unsubstituted butylene, or substituted or unsubstituted pentylene. In some embodiments, R⁸ can be substituted or unsubstituted alkyl, substituted or unsubstituted heteroalkyl, substituted or unsubstituted alkenyl, substituted or unsubstituted heteroalkenyl, —COR⁶, —C(O)OR⁶, —C(O)NR⁶R⁷, —SR⁶, —SOR⁶, —SO₂R⁶, or —SO₂NR⁶R⁷, and wherein R⁹ can be substituted or unsubstituted alkyl, substituted or unsubstituted heteroalkyl, substituted or unsubstituted alkenyl, substituted or unsubstituted heteroalkenyl, —COR⁶, —C(O)OR⁶, —C(O)NR⁶R⁷, —SR⁶, —SOR⁶, —SO₂R⁶, or —SO₂NR⁶R⁷.

Some embodiments include compounds where Y can be —O—, and W can be absent. In some embodiments wherein Y can be —O—, and W can be absent, X can be substituted or unsubstituted methylene, substituted or unsubstituted ethylene, substituted or unsubstituted propylene, substituted or unsubstituted butylene, or substituted or unsubstituted pentylene, and Z can be a bond. In some embodiments, X can be a bond, and Z can be substituted or unsubstituted methylene, substituted or unsubstituted ethylene, substituted or unsubstituted propylene, substituted or unsubstituted butylene, or substituted or unsubstituted pentylene. In some embodiments, X can be substituted or unsubstituted methylene, substituted or unsubstituted ethylene, substituted or unsubstituted propylene, substituted or unsubstituted butylene, or substituted or unsubstituted pentylene, and wherein Z can be substituted or unsubstituted methylene, substituted or unsubstituted ethylene, substituted or unsubstituted propylene, substituted or unsubstituted butylene, or substituted or unsubstituted pentylene.

In some embodiments, V can be hydrogen or substituted or unsubstituted methyl.

In some embodiments, L¹ can be —S—, —O—, —NR⁴—, substituted or unsubstituted alkylene, or substituted or unsubstituted heteroalkylene; R¹ can be substituted or unsubstituted alkyl, substituted or unsubstituted aryl, substituted or unsubstituted fused ring aryl, substituted or unsubstituted heteroaryl, or substituted or unsubstituted heterocycloalkyl; and R⁴ can be hydrogen, substituted or unsubstituted alkyl, or substituted or unsubstituted heterocycloalkyl. In some embodiments, L¹ can be —NR⁴— or substituted or unsubstituted heteroalkyl, and R¹ can be substituted or unsubstituted alkyl or substituted or unsubstituted heteroaryl. In some embodiments, L¹ can be —NR⁴—, and R¹ can be substituted alkyl having one or more substituent groups which can be substituted or unsubstituted heteroaryl or substituted or unsubstituted heterocycloalkyl. In some embodiments, R¹ can be substituted alkyl substituted by chloro-substituted thiophenyl. In some embodiments, L¹ can be substituted or unsubstituted heteroalkyl, and R¹ can be substituted or unsubstituted heteroaryl.

In some embodiments, L² can be bond, substituted or unsubstituted alkylene, —C(O)—, or —SO₂—, and R² can be hydrogen, substituted or unsubstituted alkyl, substituted or unsubstituted heteroalkyl, substituted or unsubstituted cycloalkyl, substituted or unsubstituted aryl, substituted or unsubstituted fused ring aryl, substituted or unsubstituted heteroaryl, or substituted or unsubstituted heterocycloalkyl. In some embodiments, L² can be bond, and R² is hydrogen. In some embodiments, L² can be —C(O)—, and R² can be substituted or unsubstituted alkyl, substituted or unsubstituted heteroalkyl, substituted or unsubstituted cycloalkyl, substituted or unsubstituted aryl, substituted or unsubstituted fused ring aryl, substituted or unsubstituted heteroaryl, or substituted or unsubstituted heterocycloalkyl.

In some embodiments, the compound(s) can be included among those set forth in Table A, Table B, or Table C.

Embodiments of the invention also relate to pharmaceutical compositions comprising one or more compounds as set forth above, or one or more compound(s) included among those set forth in Table A, Table B, or Table C, and a pharmaceutically acceptable excipient.

Embodiments of the invention also include methods for treating and/or preventing one or more diseases or disorders in a subject, comprising administering a compound as set forth above, or a pharmaceutical composition including such a compound, to a subject in need thereof in an amount effective to treat or prevent said disease(s) or disorder(s).

In some embodiments of the methods described herein, the disease or disorder to be treated can include one or more thrombotic diseases or disorders and/or can involve a blood clot thrombus or the potential formation of a blood clot thrombus. In some embodiments, the thrombotic disease or disorder can be acute coronary syndrome, thromboembolism, and/or thrombosis. In some embodiments, the thromboembolism can be venous thromboembolism, arterial thromboembolism, and/or cardiogenic thromboembolism. In some embodiments, the venous thromboembolism can include deep vein thrombosis and/or pulmonary embolism. In some embodiments, the deep vein thrombosis and/or pulmonary embolism can occur following a medical procedure. In some embodiments, the thrombotic disease or disorder can involve dysfunctional coagulation or disseminated intravascular coagulation. In some embodiments, the subject with dysfunctional coagulation can be undergoing percutaneous coronary intervention (PCI). In some embodiments, the thrombotic disease or disorder can involve a blood clot thrombus or the potential formation of a blood clot thrombus and further can involve stroke and/or one or more transient ischemic attacks (TIA). In some embodiments, the thrombotic disease or disorder involving a blood clot thrombus or the potential formation of a blood clot thrombus can further involve stroke, wherein the subject can have non-valvular atrial fibrillation. In some embodiments, the thrombotic disease or disorder can involve a blood clot thrombus or the potential formation of a blood clot thrombus and further can involve pulmonary hypertension. In some embodiments, the pulmonary hypertension can be caused by one or more left heart disorder and/or chronic thromboembolic disease. In some embodiments, the pulmonary hypertension can be associated with one or more lung disease, including pulmonary fibrosis (idiopathic or otherwise), and/or hypoxia.

In some embodiments, the venous thromboembolism can be associated with formation of a thrombus within a vein associated with one or more acquired or inherited risk factors and/or embolism of peripheral veins caused by a detached thrombus. In some embodiments, the one or more risk factors can include a previous venous thromboembolism. In some embodiments, the cardiogenic thromboembolism can be due to formation of a thrombus in the heart associated with cardiac arrhythmia, heart valve defect, prosthetic heart valves or heart disease, and/or embolism of peripheral arteries caused by a detached thrombus. In some embodiments, the detached thrombus can be in the brain (ischemic stroke). In some embodiments, the detached thrombus can cause a transient ischemic attack (TIA). In some embodiments, the cardiogenic thromboembolism can be due to non-valvular atrial fibrillation. In some embodiments, the thrombosis can be arterial thrombosis. In some embodiments, the arterial thrombosis can be due to one or more underlying atherosclerotic processes in the arteries. In some embodiments, the one or more underlying atherosclerotic processes in the arteries can obstruct or occlude an artery, cause myocardial ischemia (angina pectoris, acute coronary syndrome), cause myocardial infarction, obstruct or occlude a peripheral artery (ischemic peripheral artery disease), and/or obstruct or occlude the artery after a procedure on a blood vessel (reocclusion or restenosis after transluminal coronary angioplasty, reocclusion or restenosis after percutaneous transluminal angioplasty of peripheral arteries).

In some embodiments, the disease or disorder can include fibrosis, Alzheimer's Disease, multiple sclerosis, pain, cancer, inflammation, and/or Type I diabetes mellitus. In some embodiments, the disease or disorder can involve recurrent cardiac events after myocardial infarction.

In some embodiments, the treatment or prevention can include an adjunct therapy. In some embodiments, the subject can have myocardial infarction, and the adjunct therapy can be in conjunction with thrombolytic therapy. In some embodiments, the subject can have unstable angina pectoris, thrombosis, and/or heparin-induced thrombocytopenia, and the adjunct therapy can be in combination with antiplatelet therapy. In some embodiments, the subject can have non-valvular atrial fibrillation, and the adjunct therapy can be in conjunction with one or more other therapies.

In some embodiments of the methods described herein, the disease or disorder can be a kallikrein-related disorder. In some embodiments, the kallikrein-related disorder can be a thrombotic disease, a fibrinolytic disease, a fibrotic disorder, a type of cancer, an inflammatory condition, or a dermatological condition.

In some embodiments, the kallikrein-related disorder can be an ophthalmic disease. In some embodiments, the compound or pharmaceutical composition can be administered in the form of an ophthalmic composition applied topically to the eye. In some embodiments, the ophthalmic composition can be in the form of eye drops. In some embodiments, the compound or pharmaceutical composition can be administered in the form of an ophthalmic composition via intravitreal injection. In some embodiments, the ophthalmic disease can be diabetic macular edema, hereditary angioedema, age-related macular degeneration, or diabetic retinopathy.

In some embodiments wherein the disease or disorder can be a type of cancer, said type of cancer can be cervical-, testicular-, or non-small-cell lung adenocarcinoma. In some embodiments, the cancer can be limited small cell lung cancer. In some embodiments, the cancer can be a glioma. In some embodiments, the cancer can be malignant breast cancer. In some embodiments, the cancer can be a micrometastasis. In some embodiments, the micrometastasis can be of the blood or liver. In some embodiments, the cancer can be a lung metastasis. In some embodiments, the cancer can be prostatic cancer.

In some embodiments wherein the disease or disorder can be an inflammatory condition, said inflammatory condition can be sepsis, inflammatory bowel disease, systemic inflammatory response syndrome, inflammatory arthritis, or rheumatoid arthritis.

In some embodiments wherein the disease or disorder can be a dermatological condition, said dermatological condition can be atopic dermatitis, psoriasis, or Netherton Syndrome.

In some embodiments, the compound can act by inhibiting thrombin and/or kallikrein. In some embodiments, the compound can act by inhibiting tissue kallikrein and/or plasma kallikrein. In some embodiments, the compound can have inhibitory activity against thrombin and/or plasma kallikrein within a range of 1-10 nM, 10-100 nM, 0.1-1 μM, 1-10 μM, 10-100 μM, 100-200 μM, 200-500 μM, or 500-1000 μM, or greater.

In some embodiments, the amount of compound administered can be a therapeutically effective dose sufficient to achieve a plasma concentration of the compound or its active metabolite(s) within a range of 1-10 nM, 10-100 nM, 0.1-1 μM, 1-10 μM, 10-100 μM, 100-200 μM, 200-500 μM, or 500-1000 μM, or greater.

Embodiments of the invention also relate to compounds or pharmaceutical compositions as described herein, for use in methods for treating and/or preventing one or more diseases or disorders in a subject, as described herein.

### BRIEF DESCRIPTION OF THE DRAWINGS

Not applicable.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, the term “attached” signifies a stable covalent bond, certain preferred points of attachment being apparent to those of ordinary skill in the art.

The terms “halogen” or “halo” include fluorine, chlorine, bromine, and iodine. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “alkyl,” by itself or as part of another substituent, means, unless otherwise stated, a straight (i.e., unbranched) or branched chain, or combination thereof, which can be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Accordingly, the term “alkyl” can refer to C₁-C₁₆ straight chain saturated, C₁-C₁₆ branched saturated, C₃-C₈ cyclic saturated, C₃-C₈ cyclic unsaturated, and C₁-C₁₆ straight chain or branched saturated or unsaturated aliphatic hydrocarbon groups substituted with C₃-C₈ cyclic saturated or unsaturated aliphatic hydrocarbon groups having the specified number of carbon atoms, and the like. Examples of cyclic alkyl groups include, but are not limited to, cyclopropyl, cyclobutyl, cyclopentyl, cyclohexyl, cycloheptyl, cyclooctyl, cyclopropylmethyl, and the like.

The term “alkylene,” by itself or as part of another substituent, means, unless otherwise stated, a divalent radical derived from a saturated or unsaturated alkyl, as defined above and as exemplified, but not limited by, —CH₂CH₂CH₂CH₂—, and the like. Typically, an alkyl (or alkylene) group will have from 1 to 24 carbon atoms, with those groups having 10 or fewer carbon atoms being preferred in the compounds disclosed herein. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms can optionally be oxidized, and the nitrogen heteroatom can optionally be quaternized. The heteroatom(s) O, N, P, S, and Si can be placed at any interior position of the heteroalkyl group or at the position at which the alkyl group is attached to the remainder of the molecule. The heteroalkyl group can be fully saturated, mono- or polyunsaturated and can include di- and multivalent radicals, having the number of atoms designated. Accordingly, the term “heteroalkyl” can refer to saturated or unsaturated straight or branched chains containing two through 16 atoms along the chain, cyclic saturated or unsaturated groups containing 3-8 atoms in the cycle, and the like. Examples include, but are not limited

to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —C—H═CH—N(CH₃)—CH₃, —O—CH₃, —O—CH₂—CH₃, —CN, and the like. Up to two heteroatoms can be consecutive, such as, for example, —CH₂—NH—OCH₃.

Similarly, the term “heteroalkylene,” by itself or as part of another substituent, means, unless otherwise stated, a divalent radical derived from heteroalkyl, as defined above and as exemplified, but not limited by, —CH₂—CH₂—S—CH₂—CH₂— and —CH₂—S—CH₂—CH₂—NH—CH₂—, and the like. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SW, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. The “cycloalkyl” and “heterocycloalkyl” groups include, for example, monocyclic rings having 3-8 ring members, as well as bicyclic rings having 4-16 ring members, tricyclic rings having 5-24 ring members, and so on. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The term “alkenyl” includes C₂-C₁₆ straight chain unsaturated, C₂-C₁₁ branched unsaturated, C₅-C₈ unsaturated cyclic, and C₂-C₁₆ straight chain or branched unsaturated aliphatic hydrocarbon groups substituted with C₃-C₈ cyclic saturated and unsaturated aliphatic hydrocarbon groups having the specified number of carbon atoms. Double bonds can occur in any stable point along the chain and the carbon-carbon double bonds can have either the cis or trans configuration. For example, this definition shall include but is not limited to ethenyl, propenyl, butenyl, pentenyl, hexenyl, heptenyl, octenyl, nonenyl, decenyl, undecenyl, 1,5-octadienyl, 1,4,7-nonatrienyl, cyclopentenyl, cyclohexenyl, cycloheptenyl, cyclooctenyl, ethylcyclohexenyl, butenylcyclopentyl, 1-pentenyl-3-cyclohexenyl, and the like. Similarly, “heteroalkenyl” refers to heteroalkyl having one or more double bonds, wherein heteroalkyl is as defined above.

The term “alkynyl” refers in the customary sense to alkyl, as defined above, additionally having one or more triple bonds. The term “cycloalkenyl” refers to cycloalkyl, as defined above, additionally having one or more double bonds. The term “heterocycloalkenyl” refers to heterocycloalkyl, as defined above, additionally having one or more double bonds.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently, wherein each ring contains between 4-20 atoms, and preferably between 5-10 atoms. A fused ring aryl refers to multiple rings fused together wherein at least one of the fused rings is an aryl ring. The term “heteroaryl” refers to aryl groups (or rings), as defined above, that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl, and the like. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Accordingly, the term “aryl” can represent an unsubstituted, mono-, di- or trisubstituted monocyclic, polycyclic, biaryl and heterocyclic aromatic groups covalently attached at any ring position capable of forming a stable covalent bond, certain preferred points of attachment being apparent to those skilled in the art (e. g. 3-indolyl, 4-imidazolyl). The aryl substituents are independently selected from the group consisting of halo, nitro, cyano, trihalomethyl, C₁₋₁₆alkyl, arylC₁₋₁₆alkyl, C₀₋₁₆alkyloxyC₀₋₁₆alkyl, arylC₀₋₁₆alkyloxyC₀₋₁₆alkyl, C₀₋₁₆alkylthioC₀₋₁₆alkyl, arylC₀₋₁₆alkylthioC₀₋₁₆alkyl, C₀₋₁₆alkylaminoC₀₋₁₆alkyl, arylC₀₋₁₆alkylaminoC₀₋₁₆alkyl, di(arylC₁₋₁₆alkyl)aminoC₀₋₁₆alkyl, C₁₋₁₆alkylcarbonylC₀₋₁₆alkyl, arylC₁₋₁₆alkylcarbonylC₀₋₁₆alkyl, C₁₋₁₆alkylcarboxyC₀₋₁₆alkyl, arylC₁₋₁₆alkylcarboxyC₀₋₁₆alkyl, C₁₋₁₆alkylcarbonylaminoC₀₋₁₆alkyl, arylC₁₋₁₆alkylcarbonylaminoC₀₋₁₆alkyl, —C₀₋₁₆alkylCOOR₄, —C₀₋₁₆alkylCONR₅R₆ wherein R₄, R₅ and R₆ are independently selected from hydrogen, C₁-C₁₁alkyl, arylC₀-C₁₁alkyl, or R₅ and R₆ are taken together with the nitrogen to which they are attached forming a cyclic system containing 3 to 8 carbon atoms with or without one C₁₋₁₆alkyl, arylC₀-C₁₁alkyl, or C₀-Cl₁₆alkylaryl substituent. Aryl includes but is not limited to pyrazolyl and triazolyl.

For brevity, the term “aryl” when used in combination with other terms (e.g., aryloxy, arylthioxy, arylalkyl) includes both aryl and heteroaryl rings as defined above. Thus, the terms “arylalkyl,” “aralkyl” and the like are meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like), or a sulfur atom. Accordingly, the terms “arylalkyl” and the like (e.g. (4-hydroxyphenyl)ethyl, (2-aminonaphthyl)hexyl, pyridylcyclopentyl) represents an aryl group as defined above attached through an alkyl group as defined above having the indicated number of carbon atoms.

Each of the above terms (e.g., “alkyl,” “heteroalkyl,” “aryl,” and “heteroaryl”) includes both substituted and unsubstituted forms of the indicated radical. Preferred substituents for each type of radical are provided herein.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)W, —C(O)R′, —OC₂R′, —CONR′R″, —OC(O) NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, and R′″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound disclosed herein includes more than one R group, for example, each of the R groups is independently selected as are each R′, R″, and R′″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound disclosed herein includes more than one R group, for example, each of the R groups is independently selected as are each R′, R″, and R′″ groups when more than one of these groups is present.

Two or more substituents can optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring can optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring can optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed can optionally be replaced with a double bond. Alternatively, two of the substituents on adjacent atoms of the aryl or heteroaryl ring can optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

The term “alkyloxy” (e.g. methoxy, ethoxy, propyloxy, allyloxy, cyclohexyloxy) represents an alkyl group as defined above having the indicated number of carbon atoms attached through an oxygen bridge (—O—).

The term “alkylthio” (e.g. methylthio, ethylthio, propylthio, cyclohexylthio and the like) represents an alkyl group as defined above having the indicated number of carbon atoms attached through a sulfur bridge (—S—).

The term “alkylamino” represents one or two alkyl groups as defined above having the indicated number of carbon atoms attached through an amine bridge. The two alkyl groups can be taken together with the nitrogen to which they are attached forming a cyclic system containing 3 to 8 carbon atoms with or without one C₁-C₁₆alkyl, arylC₀-C₁₆alkyl, or C₀-C₁₆alkylaryl substituent.

The term “alkylaminoalkyl” represents an alkylamino group attached through an alkyl group as defined above having the indicated number of carbon atoms.

The term “alkyloxy(alkyl)amino” (e.g. methoxy(methyl)amine, ethoxy(propyl)amine) represents an alkyloxy group as defined above attached through an amino group, the amino group itself having an alkyl substituent.

The term “alkylcarbonyl” (e.g. cyclooctylcarbonyl, pentylcarbonyl, 3-hexylcarbonyl) represents an alkyl group as defined above having the indicated number of carbon atoms attached through a carbonyl group.

The term “alkylcarboxy” (e.g. heptylcarboxy, cyclopropylcarboxy, 3-pentenylcarboxy) represents an alkylcarbonyl group as defined above wherein the carbonyl is in turn attached through an oxygen.

The term “alkylcarboxyalkyl” represents an alkylcarboxy group attached through an alkyl group as defined above having the indicated number of carbon atoms.

The term “alkylcarbonylamino” (e.g. hexylcarbonylamino, cyclopentylcarbonylaminomethyl, methylcarbonylaminophenyl) represents an alkylcarbonyl group as defined above wherein the carbonyl is in turn attached through the nitrogen atom of an amino group.

The nitrogen group can itself be substituted with an alkyl or aryl group.

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ can have a specified number of carbons (e.g., “C₁-C₄ alkylsulfonyl”).

The term “carbonyloxy” represents a carbonyl group attached through an oxygen bridge.

In the above definitions, the terms “alkyl” and “alkenyl” can be used interchangeably in so far as a stable chemical entity is formed, as would be apparent to those skilled in the art.

The term “linker” refers to attachment groups interposed between substituents, e.g., R¹, R², R³ or R⁴ described herein, e.g., Formula (Ia) and generically referred to as Rⁿ, and the group which is substituted, e.g., “ring A” group of e.g., Formula (Ia). In some embodiments, the linker includes amido (—CONH—Rⁿ or —NHCO—Rⁿ), thioamido (—CSNH—Rⁿ or —NHCS—Rⁿ), carboxyl (—CO₂—Rⁿ or —OCORⁿ), carbonyl (—CO—Rⁿ), urea (—NHCONH—Rⁿ), thiourea (—NHCSNH—Rⁿ), sulfonamido (—NHSO₂—Rⁿ or —SO₂NH—Rⁿ), ether (—O—Rⁿ), sulfonyl (—SO₂—Rⁿ), sulfoxyl (—SO—Rⁿ), carbamoyl (—NHCO₂—Rⁿ or —OCONH—Rⁿ), or amino (—NHRⁿ) linking moieties.

A “substituent group,” as used herein, means a group selected from the following moieties:
(A) —OH, —NH₂, —SH, —CN, —CF₃, —NO₂, oxo, halogen, —COOH, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, —COOH, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, —COOH, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from:
      oxo, —OH, —NH₂, —SH, —CN, —CF₃, —NO₂, halogen, —COOH, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2-20-membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4-8-membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2-8-membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5-7-membered heterocycloalkyl.

The term “about” used in the context of a numeric value indicates a range of +/−10% of the numeric value, unless expressly indicated otherwise.

### II. Compounds

Embodiments of the invention encompass compounds with structure of Formula (I):

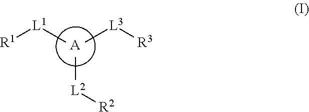

or pharmaceutical compositions including the compound and a pharmaceutically acceptable excipient, to a subject in need thereof in an amount effective to treat or prevent said disease or disorder, wherein: Ring A can be substituted or unsubstituted triazolyl; L¹ and L² can be independently a bond, substituted or unsubstituted alkylene, substituted or unsubstituted heteroalkylene, —S—, —SO—, —SO₂—, —O—, —NHSO₂—, or —NR⁴—; L³ can be a bond, substituted or unsubstituted alkylene, or substituted or unsubstituted heteroalkylene; R¹ and R² can be independently hydrogen, halogen, substituted or unsubstituted alkyl, substituted or unsubstituted heteroalkyl, substituted or unsubstituted cycloalkyl, substituted or unsubstituted cycloalkenyl, substituted or unsubstituted heterocycloalkyl, substituted or unsubstituted heterocycloalkenyl, substituted or unsubstituted aryl, substituted or unsubstituted fused ring aryl, or substituted or unsubstituted heteroaryl; R³ can be substituted or unsubstituted cycloalkyl, substituted or unsubstituted cycloalkenyl, or substituted or unsubstituted heterocycloalkyl; and R⁴ can be hydrogen, substituted or unsubstituted alkyl, substituted or unsubstituted heteroalkyl, substituted or unsubstituted alkylene, substituted or unsubstituted heteroalkylene, substituted or unsubstituted cycloalkyl, substituted or unsubstituted cycloalkenyl, substituted or unsubstituted heterocycloalkyl, substituted or unsubstituted heterocycloalkenyl, substituted or unsubstituted aryl, substituted or unsubstituted fused ring aryl, or substituted or unsubstituted heteroaryl.

In some embodiments, the compound can have the structure of Formula (II):

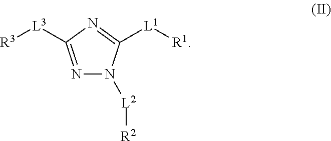

In some embodiments, L³ can be a bond or substituted or unsubstituted alkylene, and R³ can be substituted or unsubstituted cycloalkyl, substituted or unsubstituted cycloalkenyl, or substituted or unsubstituted heterocycloalkyl. In some embodiments, the cycloalkyl can be cyclopentyl, cyclohexyl, or cycloheptyl. In some embodiments, the cycloalkenyl can be cyclohexenyl. In some embodiments, the heterocycloalkyl can be azetidinyl, pyrrolidinyl, piperidinyl, oxolanyl, or oxanyl.

In some embodiments, L¹ can be —S—, —O—, —NR⁴—, substituted or unsubstituted alkylene, or substituted or unsubstituted heteroalkylene, and R¹ can be hydrogen, substituted or unsubstituted alkyl, substituted or unsubstituted aryl, substituted or unsubstituted fused ring aryl, substituted or unsubstituted heteroaryl, or substituted or unsubstituted heterocycloalkyl. In some embodiments, the heteroaryl is pyridyl, pyridazinyl, pyrimidinyl, thienyl, or furyl. In some embodiments, R¹ can be chloro-substituted thienyl. In some embodiments, R¹ can be substituted or unsubstituted phenyl, substituted or unsubstituted morpholinyl, substituted or unsubstituted oxanyl, substituted or unsubstituted oxetanyl, substituted or unsubstituted naphthyl or substituted or unsubstituted benzodioxinyl. In some embodiments, L² and R² can be absent. In some embodiments, L² can be substituted or unsubstituted alkylene or —C(O)—, and R² can be hydrogen, substituted or unsubstituted alkyl, substituted or unsubstituted heteroalkyl, substituted or unsubstituted cycloalkyl, substituted or unsubstituted aryl, substituted or unsubstituted fused ring aryl, substituted or unsubstituted heteroaryl, or substituted or unsubstituted heterocycloalkyl. In some embodiments, the heteroaryl can be pyridyl, pyridazinyl, pyrimidinyl, thienyl, or furyl. In some embodiments, R² can be substituted or unsubstituted phenyl, substituted or unsubstituted morpholinyl, substituted or unsubstituted oxanyl, substituted or unsubstituted oxetanyl, substituted or unsubstituted naphthyl or substituted or unsubstituted benzodioxinyl.

In some embodiments, L³ can be a bond, R³ can be substituted or unsubstituted cycloalkyl, substituted or unsubstituted cycloalkenyl, or substituted or unsubstituted heterocycloalkyl, and said compound can have the structure of Formula (III):

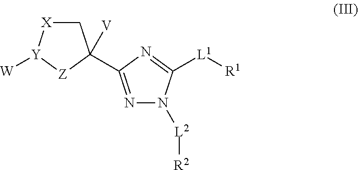

wherein: L¹ and L² are independently a bond, substituted or unsubstituted alkylene, substituted or unsubstituted heteroalkylene, —C(O)—, —S—, —SO—, —SO₂—, —O—, —NHSO₂—, —NHC(O)—, or —NR⁴—; R¹ and R² are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted fused ring aryl, or substituted or unsubstituted heteroaryl; R⁴ is hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted fused ring aryl, or substituted or unsubstituted heteroaryl; V is hydrogen or substituted or unsubstituted alkyl; W is absent, hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted heteroaryl, —C(O)R⁶, —C(O)OR⁶, —C(O)NR⁶R⁷, —SR⁶, —SOR⁶, —SO₂R⁶, —SO₂NR⁶R⁷, —OR⁶, —NHSO₂R⁶, or —NR⁶R⁷, where R⁶ and R⁷ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, or substituted or unsubstituted heteroaryl, wherein R⁶ and R⁷ can be combined if both are present to form a substituted or unsubstituted alkylene, or substituted or unsubstituted heteroalkylene; X is a bond, substituted or unsubstituted alkylene, —O—, or —NR⁸—; Y is a bond, substituted or unsubstituted alkylene, —O—, or —N—, provided that when Y is —O—, W is absent; and Z is a bond, —C(O)—, substituted or unsubstituted alkylene, —O—, or —NR⁹—; wherein R⁸ and R⁹ are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted heteroaryl, —C(O)R⁶, —C(O)OR⁶, —SR⁶, —SOR⁶, —SO₂R⁶, —SO₂NR⁶R⁷, —OR⁶, —NHSO₂R⁶, or —NR⁶R⁷, wherein R⁶ and R⁷ are as defined above; and provided that either at least one of X is —O— or —NR⁸—, Y is —O— or —N—, or Z is —O— or —NR⁹—. In some embodiments, the compound is a pharmaceutically acceptable salt, ester, solvate, or prodrug of a compound of Formula (IV). In some embodiments, the compound is not an ester, not a solvate, and not a prodrug.

In some embodiments, X is a bond or substituted or unsubstituted alkylene. In some embodiments, Z is a bond or substituted or unsubstituted alkylene. In some embodiments, X is a bond or substituted or unsubstituted alkylene, and Z is a bond or substituted or unsubstituted alkylene.

In some embodiments, wherein X is a bond or substituted or unsubstituted alkylene and Z is a bond or substituted or unsubstituted alkylene, Y is —N—, and W is hydrogen, substituted or unsubstituted alkyl, substituted or unsubstituted heteroalkyl, substituted or unsubstituted aryl, —C(O)R⁶, —C(O)OR⁶, —C(O)NR⁶R⁷′—SO₂R⁶, or SO₂NR⁶R⁷, wherein R⁶ and R⁷ are independently substituted or unsubstituted alkyl, substituted or unsubstituted cycloalkyl, or substituted or unsubstituted heterocycloalkyl, or R⁶ and R⁷ can be combined if both are present to form a substituted or unsubstituted alkylene, or substituted or unsubstituted heteroalkylene.

In some embodiments, X is selected from the group consisting of substituted or unsubstituted methylene, substituted or unsubstituted ethylene, substituted or unsubstituted propylene, substituted or unsubstituted butylene, and substituted or unsubstituted pentylene, and Z is a bond. In some embodiments, X is a bond, and Z is selected from the group consisting of substituted or unsubstituted methylene, substituted or unsubstituted ethylene, substituted or unsubstituted propylene, substituted or unsubstituted butylene, and substituted or unsubstituted pentylene. In some embodiments, X is selected from the group consisting of substituted or unsubstituted methylene, substituted or unsubstituted ethylene, substituted or unsubstituted propylene, substituted or unsubstituted butylene, and substituted or unsubstituted pentylene, and wherein Z is selected from the group consisting of substituted or unsubstituted methylene, substituted or unsubstituted ethylene, substituted or unsubstituted propylene, substituted or unsubstituted butylene, and substituted or unsubstituted pentylene. In some embodiments, X and Z are both branched alkylene and X and Z are covalently attached. In some embodiments, Z is can be substituted methylene, substituted ethylene, substituted propylene, substituted butylene, or substituted pentylene, having one or more substituent groups selected from the group consisting of —OH, —NH₂, —SH, —CN, —CF₃, —NO₂, oxo, halogen, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, X is selected from the group consisting of substituted or unsubstituted methylene, substituted or unsubstituted ethylene, substituted or unsubstituted propylene, substituted or unsubstituted butylene, and substituted or unsubstituted pentylene, and wherein Z is —C(O)—. In some embodiments, W is hydrogen. In some embodiments, W is selected from the group consisting of substituted or unsubstituted alkyl, substituted or unsubstituted heteroalkyl, substituted or unsubstituted alkenyl, substituted or unsubstituted heteroalkenyl, substituted or unsubstituted cycloalkyl, substituted or unsubstituted heterocycloalkyl, —C(O)R⁶, —C(O)OR⁶, —C(O)NR⁶R⁷′—SR⁶, —SOR⁶, —SO₂R⁶, and —SO₂NR⁶. In some embodiments, W is substituted alkyl, substituted heteroalkyl, substituted alkenyl, substituted heteroalkenyl, substituted cycloalkyl, or substituted heterocycloalkyl, having one or more substituent groups selected from the group consisting of —OH, —NH₂, —SH, —CN, —CF₃, —NO₂, oxo, halogen, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, W is —COR⁶, —C(O)OR⁶, —C(O)NR⁶R⁷, —SO₂R⁶ or —SO₂NR⁶R⁷, and wherein R⁶ and R⁷ are selected from the group consisting of substituted or unsubstituted alkyl, substituted or unsubstituted heteroalkyl, substituted or unsubstituted aryl, and substituted or unsubstituted heteroaryl, or R⁶ and R⁷ combine to form a substituted or unsubstituted alkylene.

In some embodiments, W is absent, X is —NR^{B}—, Y is a bond or substituted or unsubstituted alkylene, and Z is —NR⁹—. In some embodiments wherein W is absent, X is —NR⁸—, Y is a bond or substituted or unsubstituted alkylene, and Z is —NR⁹—, Y is selected from the group consisting of substituted or unsubstituted methylene, substituted or unsubstituted ethylene, substituted or unsubstituted propylene, substituted or unsubstituted butylene, and substituted or unsubstituted pentylene. In some embodiments, R⁸ is selected from the group consisting of substituted or unsubstituted alkyl, substituted or unsubstituted heteroalkyl, substituted or unsubstituted alkenyl, substituted or unsubstituted heteroalkenyl, —COR⁶, —C(O)OR⁶, —C(O)NR⁶R⁷, —SR⁶, —SOR⁶, —SO₂R⁶, and —SO₂NR⁶R⁷, and wherein R⁹ is selected from the group consisting of substituted or unsubstituted alkyl, substituted or unsubstituted heteroalkyl, substituted or unsubstituted alkenyl, substituted or unsubstituted heteroalkenyl, —COR⁶, —C(O)OR⁶, —C(O)NR⁶R⁷, —SR⁶, —SOR⁶, —SO₂R⁶, and —SO₂NR⁶R⁷.

In some embodiments, Y is —O—, and W is absent. In some embodiments wherein Y is —O—, and W is absent, X is selected from the group consisting of substituted or unsubstituted methylene, substituted or unsubstituted ethylene, substituted or unsubstituted propylene, substituted or unsubstituted butylene, and substituted or unsubstituted pentylene, and Z is a bond. In some embodiments, X is a bond, and Z is selected from the group consisting of substituted or unsubstituted methylene, substituted or unsubstituted ethylene, substituted or unsubstituted propylene, substituted or unsubstituted butylene, and substituted or unsubstituted pentylene. In some embodiments, X is selected from the group consisting of substituted or unsubstituted methylene, substituted or unsubstituted ethylene, substituted or unsubstituted propylene, substituted or unsubstituted butylene, and substituted or unsubstituted pentylene, and wherein Z is selected from the group consisting of substituted or unsubstituted methylene, substituted or unsubstituted ethylene, substituted or unsubstituted propylene, substituted or unsubstituted butylene, and substituted or unsubstituted pentylene.

In some embodiments, V is hydrogen or substituted or unsubstituted methyl.

Further to any embodiment above wherein the compound has the structure of Formula (IV), in some embodiments, L¹ is —S—, —O—, —NR⁴—, substituted or unsubstituted alkylene, or substituted or unsubstituted heteroalkylene; R¹ is substituted or unsubstituted alkyl, substituted or unsubstituted aryl, substituted or unsubstituted fused ring aryl, substituted or unsubstituted heteroaryl, or substituted or unsubstituted heterocycloalkyl; and R⁴ is hydrogen, substituted or unsubstituted alkyl, or substituted or unsubstituted heterocycloalkyl. In some embodiments, L¹ is —NR⁴— or substituted or unsubstituted heteroalkyl, and R¹ is substituted or unsubstituted alkyl or substituted or unsubstituted heteroaryl. In some embodiments, L¹ is —NR⁴—, and R¹ is substituted alkyl having one or more substituent groups selected from the group consisting of substituted or unsubstituted heteroaryl and substituted or unsubstituted heterocycloalkyl. In some embodiments, R¹ is substituted alkyl substituted by chloro-substituted thiophenyl. In some embodiments, L¹ is substituted or unsubstituted heteroalkyl, and R¹ is substituted or unsubstituted heteroaryl. In some embodiments, L¹ and R¹ can be any specific group as set forth above for any of Formulae (I) or (II).

Further to any embodiment above wherein the compound has the structure of Formula (IV), in some embodiments, L² is bond, substituted or unsubstituted alkylene, —C(O)—, or —SO₂—, and R² is hydrogen, substituted or unsubstituted alkyl, substituted or unsubstituted heteroalkyl, substituted or unsubstituted cycloalkyl, substituted or unsubstituted aryl, substituted or unsubstituted fused ring aryl, substituted or unsubstituted heteroaryl, or substituted or unsubstituted heterocycloalkyl. In some embodiments, L² is bond, and R² is hydrogen. In some embodiments, L² is —C(O)—, and R² is substituted or unsubstituted alkyl, substituted or unsubstituted heteroalkyl, substituted or unsubstituted cycloalkyl, substituted or unsubstituted aryl, substituted or unsubstituted fused ring aryl, substituted or unsubstituted heteroaryl, or substituted or unsubstituted heterocycloalkyl. In some embodiments, L² and R² can be any specific group as set forth above for any of Formulae (I) or (II). In some embodiments, the compound can be among those listed in Table A, Table B, or Table C.

Exemplary compounds, e.g., multisubstituted aromatic compounds, in accordance with the present disclosure are provided herein. In Table A and B following, entry number, chemical name (i.e., International Union of Pure and Applied Chemistry [IUPAC] name), calculated molecular weight (MW) and biological activity (i.e., inhibition activity in thrombin and KLKB1 assays) are disclosed. In Table C following, chemical names are disclosed.

For Table A following, the disclosed compounds were assayed for inhibition of the protease activity of thrombin as described herein. In Table A, the level of inhibition in the thrombin assay is indicated as follows: a IC₅₀≦0.1 μM; b: 0.1 μM<IC₅₀<1 μM; c: 1 μM<IC₅₀<10 μM; d: 10 μM<IC₅₀<100 μM; e: IC₅₀≧100 μM. Accordingly, in some embodiments, there is provided a compound as expressly set forth in Table A following.

TABLE A

| | | | Thrombin |
|---|---|---|---|
| Entry | IUPAC name | MW | Activity |
| 1 | 1-(5-[(5-chlorothiophen-2-yl)methyl]amino-3-(oxan-4- | 383 | a |
| | yl)-1H-1,2,4-triazol-1-yl)-2,2-dimethylpropan-1-one | | |
| 2 | 1-(5-[(5-chlorothiophen-2-yl)methyl]amino-3-(oxolan-2- | 369 | a |
| | yl)-1H-1,2,4-triazol-1-yl)-2,2-dimethylpropan-1-one | | |
| 3 | 1-(5-[(5-chlorothiophen-2-yl)methyl]amino-3-(oxolan-3- | 369 | a |
| | yl)-1H-1,2,4-triazol-1-yl)-2,2-dimethylpropan-1-one | | |
| 4 | 1-(5-[(5-chlorothiophen-2-yl)methyl]amino-3-(piperidin- | 412 | a |
| | 4-yl)-1H-1,2,4-triazol-1-yl)-3-methoxy-2,2- | | |
| | dimethylpropan-1-one | | |
| 5 | 1-[2-(5-[(5-chlorothiophen-2-yl)methyl]amino-1-(2- | 502 | a |
| | methoxybenzoyl)-1H-1,2,4-triazol-3-yl)pyrrolidin-1-yl]- | | |
| | 2,2-dimethylpropan-1-one | | |
| 6 | 1-[2-(5-[(5-chlorothiophen-2-yl)methyl]amino-1-(2- | 488 | a |
| | methoxybenzoyl)-1H-1,2,4-triazol-3-yl)pyrrolidin-1-yl]- | | |
| | 2-methylpropan-1-one | | |
| 7 | 1-[2-(5-[(5-chlorothiophen-2-yl)methyl]amino-1-(2- | 460 | a |
| | methoxybenzoyl)-1H-1,2,4-triazol-3-yl)pyrrolidin-1- | | |
| | yl]ethan-1-one | | |
| 8 | 1-[3-(5-[(5-chlorothiophen-2-yl)methyl]amino-1-(2- | 488 | a |
| | methoxybenzoyl)-1H-1,2,4-triazol-3-yl)azetidin-1-yl]- | | |
| | 2,2-dimethylpropan-1-one | | |
| 9 | 1-[3-(5-[(5-chlorothiophen-2-yl)methyl]amino-1-(2- | 446 | a |
| | methoxybenzoyl)-1H-1,2,4-triazol-3-yl)azetidin-1- | | |
| | yl]ethan-1-one | | |
| 10 | 1-[3-(5-[(5-chlorothiophen-2-yl)methyl]amino-1H-1,2,4- | 312 | c |
| | triazol-3-yl)azetidin-1-yl]ethan-1-one | | |
| 11 | 1-[4-(5-[(5-chlorothiophen-2-yl)methyl]amino-1-(2- | 516 | a |
| | methoxybenzoyl)-1H-1,2,4-triazol-3-yl)piperidin-1-yl]- | | |
| | 2,2-dimethylpropan-1-one | | |
| 12 | 1-[4-(5-[(5-chlorothiophen-2-yl)methyl]amino-1-(2- | 474 | a |
| | methoxybenzoyl)-1H-1,2,4-triazol-3-yl)piperidin-1- | | |
| | yl]ethan-1-one | | |
| 13 | 1-[4-(5-[(5-chlorothiophen-2-yl)methyl]amino-1H-1,2,4- | 382 | e |
| | triazol-3-yl)piperidin-1-yl]-2,2-dimethylpropan-1-one | | |
| 14 | 1-[4-(5-[(5-chlorothiophen-2-yl)methyl]amino-1H-1,2,4- | 340 | e |
| | triazol-3-yl)piperidin-1-yl]ethan-1-one | | |
| 15 | 1-benzoyl-N-[(5-chlorothiophen-2-yl)methyl]-3- | 402 | a |
| | (piperidin-4-yl)-1H-1,2,4-triazol-5-amine hydrochloride | | |
| 16 | 2-[5-(benzylamino)-1-(2-methoxybenzoyl)-1H-1,2,4- | 404 | e |
| | triazol-3-yl]cyclohex-1-en-1-ol | | |
| 17 | 2-[5-(benzylamino)-1H-1,2,4-triazol-3-yl]cyclohex-1-en- | 270 | e |
| | 1-ol | | |
| 18 | 3-(2-methoxycyclohexyl)-1H-1,2,4-triazol-5-amine | 196 | e |
| 19 | 3-(5-[(5-chlorothiophen-2-yl)methyl]amino-1-(2- | 511 | a |
| | methoxybenzoyl)-1H-1,2,4-triazol-3-yl)-N,N- | | |
| | dimethylazetidine-1-sulfonamide | | |
| 20 | 3-(azetidin-3-yl)-1-benzoyl-N-[(5-chlorothiophen-2- | 374 | e |
| | yl)methyl]-1H-1,2,4-triazol-5-amine | | |
| 21 | 3-(azetidin-3-yl)-N-[(5-chlorothiophen-2-yl)methyl]-1- | 432 | a |
| | (2,3-dihydro-1,4-benzodioxine-5-carbonyl)-1H-1,2,4- | | |
| | triazol-5-amine; trifluoroacetic acid | | |
| 22 | 3-(azetidin-3-yl)-N-[(5-chlorothiophen-2-yl)methyl]-1- | 380 | b |
| | cyclohexanecarbonyl-1H-1,2,4-triazol-5-amine; | | |
| | trifluoroacetic acid | | |
| 23 | 3-(azetidin-3-yl)-N-[(5-chlorothiophen-2-yl)methyl]-1- | 366 | e |
| | cyclopentanecarbonyl-1H-1,2,4-triazol-5-amine | | |
| 24 | 3-(oxan-4-yl)-1H-1,2,4-triazol-5-amine | 168 | e |
| 25 | 3-(oxolan-2-yl)-1H-1,2,4-triazol-5-amine | 154 | e |
| 26 | 3-(oxolan-3-yl)-1H-1,2,4-triazol-5-amine | 154 | e |
| 27 | 3-[1-(benzenesulfonyl)azetidin-3-yl]-N-[(5- | 544 | a |
| | chlorothiophen-2-yl)methyl]-1-(2-methoxybenzoyl)-1H- | | |
| | 1,2,4-triazol-5-amine | | |
| 28 | 3-[1-(benzenesulfonyl)piperidin-4-yl]-N-[(5- | 572 | a |
| | chlorothiophen-2-yl)methyl]-1-(2-methoxybenzoyl)-1H- | | |
| | 1,2,4-triazol-5-amine | | |
| 29 | 3-2-[(5-chlorothiophen-2-yl)methoxy]cyclohex-1-en-1- | 311 | e |
| | yl-1H-1,2,4-triazol-5-amine | | |
| 30 | 4-(5-[(5-chlorothiophen-2-yl)methyl]amino-1-(2- | 503 | a |
| | methoxybenzoyl)-1H-1,2,4-triazol-3-yl)-N,N- | | |
| | dimethylpiperidine-1-carboxamide | | |
| 31 | 4-(5-[(5-chlorothiophen-2-yl)methyl]amino-1-(2- | 475 | a |
| | methoxybenzoyl)-1H-1,2,4-triazol-3-yl)piperidine-1- | | |
| | carboxamide | | |
| 32 | N-[(5-chlorothiophen-2-yl)methyl]-1-(2,3-dihydro-1,4- | 461 | a |
| | benzodioxine-5-carbonyl)-3-(oxan-4-yl)-1H-1,2,4- | | |
| | triazol-5-amine | | |
| 33 | N-[(5-chlorothiophen-2-yl)methyl]-1-(2,3-dihydro-1,4- | 447 | a |
| | benzodioxine-5-carbonyl)-3-(oxolan-2-yl)-1H-1,2,4- | | |
| | triazol-5-amine | | |
| 34 | N-[(5-chlorothiophen-2-yl)methyl]-1-(2,3-dihydro-1,4- | 447 | a |
| | benzodioxine-5-carbonyl)-3-(oxolan-3-yl)-1H-1,2,4- | | |
| | triazol-5-amine | | |
| 35 | N-[(5-chlorothiophen-2-yl)methyl]-1-(2,3-dihydro-1,4- | 460 | a |
| | benzodioxine-5-carbonyl)-3-(piperidin-4-yl)-1H-1,2,4- | | |
| | triazol-5-amine hydrochloride | | |
| 36 | N-[(5-chlorothiophen-2-yl)methyl]-1-(2,4- | 463 | a |
| | dimethoxybenzoyl)-3-(oxan-4-yl)-1H-1,2,4-triazol-5- | | |
| | amine | | |
| 37 | N-[(5-chlorothiophen-2-yl)methyl]-1-(2,4- | 449 | a |
| | dimethoxybenzoyl)-3-(oxolan-2-yl)-1H-1,2,4-triazol-5- | | |
| | amine | | |
| 38 | N-[(5-chlorothiophen-2-yl)methyl]-1-(2,4- | 449 | a |
| | dimethoxybenzoyl)-3-(oxolan-3-yl)-1H-1,2,4-triazol-5- | | |
| | amine | | |
| 39 | N-[(5-chlorothiophen-2-yl)methyl]-1-(2- | 461 | a |
| | methoxybenzoyl)-3-(2-methoxycyclohexyl)-1H-1,2,4- | | |
| | triazol-5-amine | | |
| 40 | N-[(5-chlorothiophen-2-yl)methyl]-1-(2- | 433 | a |
| | methoxybenzoyl)-3-(oxan-4-yl)-1H-1,2,4-triazol-5- | | |
| | amine | | |
| 41 | N-[(5-chlorothiophen-2-yl)methyl]-1-(2- | 419 | a |
| | methoxybenzoyl)-3-(oxolan-2-yl)-1H-1,2,4-triazol-5- | | |
| | amine | | |
| 42 | N-[(5-chlorothiophen-2-yl)methyl]-1-(2- | 419 | a |
| | methoxybenzoyl)-3-(oxolan-3-yl)-1H-1,2,4-triazol-5- | | |
| | amine | | |
| 43 | N-[(5-chlorothiophen-2-yl)methyl]-1-(2- | 432 | a |
| | methoxybenzoyl)-3-(piperidin-4-yl)-1H-1,2,4-triazol-5- | | |
| | amine | | |
| 44 | N-[(5-chlorothiophen-2-yl)methyl]-1-(2- | 543 | a |
| | methoxybenzoyl)-3-[1-(piperidine-1-carbonyl)piperidin- | | |
| | 4-yl]-1H-1,2,4-triazol-5-amine | | |
| 45 | N-[(5-chlorothiophen-2-yl)methyl]-1-(2- | 551 | a |
| | methoxybenzoyl)-3-[1-(piperidine-1-sulfonyl)azetidin-3- | | |
| | yl]-1H-1,2,4-triazol-5-amine | | |
| 46 | N-[(5-chlorothiophen-2-yl)methyl]-1-(2- | 510 | a |
| | methoxybenzoyl)-3-[1-(propane-2-sulfonyl)azetidin-3- | | |
| | yl]-1H-1,2,4-triazol-5-amine | | |
| 47 | N-[(5-chlorothiophen-2-yl)methyl]-1-(2- | 538 | a |
| | methoxybenzoyl)-3-[1-(propane-2-sulfonyl)piperidin-4- | | |
| | yl]-1H-1,2,4-triazol-5-amine | | |
| 48 | N-[(5-chlorothiophen-2-yl)methyl]-1-(2- | 529 | a |
| | methoxybenzoyl)-3-[1-(pyrrolidine-1-carbonyl)piperidin- | | |
| | 4-yl]-1H-1,2,4-triazol-5-amine | | |
| 49 | N-[(5-chlorothiophen-2-yl)methyl]-1-(2- | 537 | a |
| | methoxybenzoyl)-3-[1-(pyrrolidine-1-sulfonyl)azetidin- | | |
| | 3-yl]-1H-1,2,4-triazol-5-amine | | |
| 50 | N-[(5-chlorothiophen-2-yl)methyl]-1-(2-methylbenzoyl)- | 417 | a |
| | 3-(oxan-4-yl)-1H-1,2,4-triazol-5-amine | | |
| 51 | N-[(5-chlorothiophen-2-yl)methyl]-1-(furan-3-carbonyl)- | 393 | a |
| | 3-(oxan-4-yl)-1H-1,2,4-triazol-5-amine | | |
| 52 | N-[(5-chlorothiophen-2-yl)methyl]-1-(furan-3-carbonyl)- | 379 | a |
| | 3-(oxolan-2-yl)-1H-1,2,4-triazol-5-amine | | |
| 53 | N-[(5-chlorothiophen-2-yl)methyl]-1-(furan-3-carbonyl)- | 379 | a |
| | 3-(oxolan-3-yl)-1H-1,2,4-triazol-5-amine | | |
| 54 | N-[(5-chlorothiophen-2-yl)methyl]-3-(1- | 482 | a |
| | methanesulfonylazetidin-3-yl)-1-(2-methoxybenzoyl)- | | |
| | 1H-1,2,4-triazol-5-amine | | |
| 55 | N-[(5-chlorothiophen-2-yl)methyl]-3-(1- | 510 | a |
| | methanesulfonylpiperidin-4-yl)-1-(2-methoxybenzoyl)- | | |
| | 1H-1,2,4-triazol-5-amine | | |
| 56 | N-[(5-chlorothiophen-2-yl)methyl]-3-(2- | 327 | c |
| | methoxycyclohexyl)-1H-1,2,4-triazol-5-amine | | |
| 57 | N-[(5-chlorothiophen-2-yl)methyl]-3-(oxan-4-yl)-1- | 409 | a |
| | (thiophene-3-carbonyl)-1H-1,2,4-triazol-5-amine | | |
| 58 | N-[(5-chlorothiophen-2-yl)methyl]-3-(oxan-4-yl)-1H- | 299 | e |
| | 1,2,4-triazol-5-amine | | |
| 59 | N-[(5-chlorothiophen-2-yl)methyl]-3-(oxolan-2-yl)-1- | 395 | a |
| | (thiophene-3-carbonyl)-1H-1,2,4-triazol-5-amine | | |
| 60 | N-[(5-chlorothiophen-2-yl)methyl]-3-(oxolan-2-yl)- | 285 | e |
| | 1H-1,2,4-triazol-5-amine | | |
| 61 | N-[(5-chlorothiophen-2-yl)methyl]-3-(oxolan-3-yl)-1- | 395 | a |
| | (thiophene-3-carbonyl)-1H-1,2,4-triazol-5-amine | | |
| 62 | N-[(5-chlorothiophen-2-yl)methyl]-3-(oxolan-3-yl)-1H- | 285 | e |
| | 1,2,4-triazol-5-amine | | |
| 63 | N-[(5-chlorothiophen-2-yl)methyl]-3-[1- | 550 | a |
| | (cyclohexanesulfonyl)azetidin-3-yl]-1-(2- | | |
| | methoxybenzoyl)-1H-1,2,4-triazol-5-amine | | |
| 64 | N-[(5-chlorothiophen-2-yl)methyl]-3-[1- | 578 | a |
| | (cyclohexanesulfonyl)piperidin-4-yl]-1-(2- | | |
| | methoxybenzoyl)-1H-1,2,4-triazol-5-amine | | |
| 65 | N-benzyl-1-(2-methoxybenzoyl)-3-(oxan-4-yl)-1H-1,2,4- | 392 | c |
| | triazol-5-amine | | |
| 66 | N-benzyl-3-(oxan-4-yl)-1H-1,2,4-triazol-5-amine | 258 | e |
| 67 | tert-butyl 2-(5-amino-1H-1,2,4-triazol-3-yl)pyrrolidine- | 253 | e |
| | 1-carboxylate | | |
| 68 | tert-butyl 2-(5-[(5-chlorothiophen-2-yl)methyl]amino-1- | 518 | b |
| | (2-methoxybenzoyl)-1H-1,2,4-triazol-3-yl)pyrrolidine-1- | | |
| | carboxylate | | |
| 69 | tert-butyl 2-(5-[(5-chlorothiophen-2-yl)methyl]amino- | 384 | e |
| | 1H-1,2,4-triazol-3-yl)pyrrolidine-1-carboxylate | | |
| 70 | tert-butyl 3-(5-amino-1H-1,2,4-triazol-3-yl)azetidine-1- | 239 | e |
| | carboxylate | | |
| 71 | tert-butyl 3-(5-[(5-chlorothiophen-2-yl)methyl]amino-1- | 504 | a |
| | (2-methoxybenzoyl)-1H-1,2,4-triazol-3-yl)azetidine-1- | | |
| | carboxylate | | |
| 72 | tert-butyl 3-(5-[(5-chlorothiophen-2-yl)methyl]amino- | 370 | e |
| | 1H-1,2,4-triazol-3-yl)azetidine-1-carboxylate | | |
| 73 | tert-butyl 4-(5-amino-1H-1,2,4-triazol-3-yl)piperidine-1- | 267 | e |
| | carboxylate | | |
| 74 | tert-butyl 4-(5-[(5-chlorothiophen-2-yl)methyl]amino-1- | 532 | a |
| | (2-methoxybenzoyl)-1H-1,2,4-triazol-3-yl)piperidine-1- | | |
| | carboxylate | | |
| 75 | tert-butyl 4-(5-[(5-chlorothiophen-2-yl)methyl]amino- | 398 | e |
| | 1H-1,2,4-triazol-3-yl)piperidine-1-carboxylate | | |
| 76 | tert-butyl 4-[5-(benzylamino)-1-(2-methoxybenzoyl)-1H- | 492 | a |
| | 1,2,4-triazol-3-yl]piperidine-1-carboxylate | | |

For Table B following, the disclosed compounds were assayed for inhibition of the protease activity of KLKB1 as described herein. In Table B following, the level of inhibition in the KLKB1 assay is indicated as follows: a: IC₅₀≦1 μM; b: 1 μM<IC₅₀<10 μM; c: IC₅₀≧10 μM. Accordingly, in some embodiments, there is provided a compound as expressly set forth in Table B following.

TABLE B

| | | | KLKB1 |
|---|---|---|---|
| Entry | IUPAC name | MW | Activity |
| 1 | 1-(5-[(5-chlorothiophen-2-yl)methyl]amino-3-(oxolan-2- | 369 | c |
| | yl)-1H-1,2,4-triazol-1-yl)-2,2-dimethylpropan-1-one | | |
| 2 | 1-[4-(5-[(5-chlorothiophen-2-yl)methyl]amino-1-(2- | 516 | b |
| | methoxybenzoyl)-1H-1,2,4-triazol-3-yl)piperidin-1-yl]- | | |
| | 2,2-dimethylpropan-1-one | | |
| 3 | 3-(5-[(5-chlorothiophen-2-yl)methyl]amino-1-(2- | 511 | a |
| | methoxybenzoyl)-1H-1,2,4-triazol-3-yl)-N,N- | | |
| | dimethylazetidine-1-sulfonamide | | |
| 4 | N-[(5-chlorothiophen-2-yl)methyl]-1-(2,4- | 463 | b |
| | dimethoxybenzoyl)-3-(oxan-4-yl)-1H-1,2,4-triazol-5- | | |
| | amine | | |
| 5 | N-[(5-chlorothiophen-2-yl)methyl]-1-(2- | 433 | b |
| | methoxybenzoyl)-3-(oxan-4-yl)-1H-1,2,4-triazol-5- | | |
| | amine | | |
| 6 | N-[(5-chlorothiophen-2-yl)methyl]-1-(2- | 419 | b |
| | methoxybenzoyl)-3-(oxolan-2-yl)-1H-1,2,4-triazol-5- | | |
| | amine | | |
| 7 | N-[(5-chlorothiophen-2-yl)methyl]-1-(2- | 432 | a |
| | methoxybenzoyl)-3-(piperidin-4-yl)-1H-1,2,4-triazol-5- | | |
| | amine | | |
| 8 | N-[(5-chlorothiophen-2-yl)methyl]-1-(2- | 537 | a |
| | methoxybenzoyl)-3-[1-(pyrrolidine-1-sulfonyl)azetidin- | | |
| | 3-yl]-1H-1,2,4-triazol-5-amine | | |
| 9 | N-[(5-chlorothiophen-2-yl)methyl]-1-(furan-3-carbonyl)- | 379 | b |
| | 3-(oxolan-2-yl)-1H-1,2,4-triazol-5-amine | | |
| 10 | N-[(5-chlorothiophen-2-yl)methyl]-1-(furan-3-carbonyl)- | 379 | b |
| | 3-(oxolan-3-yl)-1H-1,2,4-triazol-5-amine | | |
| 11 | N-[(5-chlorothiophen-2-yl)methyl]-3-(oxolan-2-yl)-1- | 395 | a |
| | (thiophene-3-carbonyl)-1H-1,2,4-triazol-5-amine | | |
| 12 | N-[(5-chlorothiophen-2-yl)methyl]-3-(oxolan-3-yl)-1- | 395 | a |
| | (thiophene-3-carbonyl)-1H-1,2,4-triazol-5-amine | | |
| 13 | tert-butyl 3-(5-[(5-chlorothiophen-2-yl)methyl]amino-1- | 504 | b |
| | (2-methoxybenzoyl)-1H-1,2,4-triazol-3-yl)azetidine-1- | | |
| | carboxylate | | |
| 14 | tert-butyl 4-(5-[(5-chlorothiophen-2-yl)methyl]amino-1- | 532 | b |
| | (2-methoxybenzoyl)-1H-1,2,4-triazol-3-yl)piperidine-1- | | |
| | carboxylate | | |

In some embodiments, there is provided a compound as expressly set forth in Table C following.

TABLE C
1-(2,2-dimethylpropanoyl)-3-(5-{[(4-fluorophenyl)methyl]amino}-1-(furan-3-carbonyl)-
1H-1,2,4-triazol-3-yl)-4-(trifluoromethyl)piperidine-2-carboxylic acid
1-(2,2-dimethylpropanoyl)-4-(5-{[(4-fluorophenyl)methyl](methyl)amino}-1-(3-methoxy-
2,2-dimethylpropanoyl)-1H-1,2,4-triazol-3-yl)-3-methylpyrrolidin-2-one
1-(2-chlorobenzoyl)-N-[(4-fluorophenyl)methyl]-3-[1-(pyrrolidine-1-carbonyl)piperidin-3-
yl]-1H-1,2,4-triazol-5-amine
1-(2-chlorobenzoyl)-N-[(4-fluorophenyl)methyl]-N-methyl-3-(piperidin-4-yl)-1H-1,2,4-
triazol-5-amine
1-(2-chlorobenzoyl)-N-[(4-fluorophenyl)methyl]-N-methyl-3-[1-(morpholine-4-
carbonyl)piperidin-3-yl]-1H-1,2,4-triazol-5-amine
1-(2-chlorobenzoyl)-N-[(5-chlorothiophen-2-yl)methyl]-3-(piperazin-2-yl)-1H-1,2,4-
triazol-5-amine
1-(2-chlorobenzoyl)-N-[(5-chlorothiophen-2-yl)methyl]-3-[1-(pyrrolidine-1-
sulfonyl)piperidin-3-yl]-1H-1,2,4-triazol-5-amine
1-(2-chlorobenzoyl)-N-[(5-chlorothiophen-2-yl)methyl]-3-[4-(pyrrolidine-1-
sulfonyl)piperazin-2-yl]-1H-1,2,4-triazol-5-amine
1-(3-{1-benzoyl-5-[(5-chlorothiophen-2-yl)methoxy]-1H-1,2,4-triazol-3-yl}piperazine-1-
carbonyl)pyrrolidin-3-ol
1-(3-{5-[(4-fluorophenyl)methoxy]-1-(1,3-thiazole-4-carbonyl)-1H-1,2,4-triazol-3-yl}-2-
methylpiperidine-1-carbonyl)pyrrolidin-3-ol
1-(3-{5-[(4-fluorophenyl)methoxy]-1H-1,2,4-triazol-3-yl}-2-methylpyrrolidin-1-yl)-2,2-
dimethylpropan-1-one
1-(3-{5-[(5-chlorothiophen-2-yl)methoxy]-1-(thiophene-2-carbonyl)-1H-1,2,4-triazol-3-
yl}piperazin-1-yl)-2-(morpholin-4-yl)ethan-1-one
1-(5-{[(4-fluorophenyl)methyl](methyl)amino}-3-(4-methyloxolan-3-yl)-1H-1,2,4-triazol-
l-yl)-3-methoxy-2,2-dimethylpropan-1-one
1-(5-{[(4-fluorophenyl)methyl](methyl)amino}-3-[5-hydroxy-1-(3-hydroxypyrrolidine-1-
carbonyl)-2-methylpyrrolidin-3-yl]-1H-1,2,4-triazol-1-yl)-3-methoxy-2,2-dimethylpropan-
1-one
1-(5-{[(4-fluorophenyl)methyl]amino}-3-{1-[(3-hydroxypyrrolidin-1-yl)sulfonyl]-2-
methylazetidin-3-yl}-1H-1,2,4-triazol-1-yl)-3-methoxy-2,2-dimethylpropan-1-one
1-(5-{[(4-fluorophenyl)methyl]sulfanyl}-3-[1-(morpholine-4-carbonyl)-3-
(trifluoromethyl)piperidin-4-yl]-1H-1,2,4-triazol-1-yl)-2,2-dimethylpropan-1-one
1-(5-{[(4-fluorophenyl)methyl]sulfanyl}-3-[1-(pyrrolidine-1-carbonyl)-3-
(trifluoromethyl)piperidin-4-yl]-1H-1,2,4-triazol-1-yl)-2,2-dimethylpropan-1-one
1-(5-{[(4-fluorophenyl)methyl]sulfanyl}-3-{1-[2-(morpholin-4-yl)acetyl]-3-
(trifluoromethyl)piperazin-2-yl}-1H-1,2,4-triazol-1-yl)-2,2-dimethylpropan-1-one
1-(5-{[(5-chlorothiophen-2-yl)methyl](methyl)amino}-3-[1-(2,2-dimethylpropanoyl)-4-
methylpiperidin-3-yl]-1H-1,2,4-triazol-1-yl)-3-hydroxy-2,2-dimethylpropan-1-one
1-(5-{[(5-chlorothiophen-2-yl)methyl](methyl)amino}-3-[1-(3-hydroxypyrrolidine-1-
carbonyl)-3-(trifluoromethyl)piperazin-2-yl]-1H-1,2,4-triazol-1-yl)-2,2-dimethylpropan-1-
one
1-(5-{[(5-chlorothiophen-2-yl)methyl](methyl)amino}-3-[1-methanesulfonyl-3-
(trifluoromethyl)piperazin-2-yl]-1H-1,2,4-triazol-1-yl)-2,2-dimethylpropan-1-one
1-(5-{[(5-chlorothiophen-2-yl)methyl](methyl)amino}-3-{1-[(3-hydroxypyrrolidin-1-
yl)sulfonyl]-2-methylazetidin-3-yl}-1H-1,2,4-triazol-1-yl)-3-hydroxy-2,2-dimethylpropan-
1-one
1-(5-{[(5-chlorothiophen-2-yl)methyl]amino}-3-[4-methyl-1-(pyrrolidine-1-
sulfonyl)pyrrolidin-3-yl]-1H-1,2,4-triazol-1-yl)-3-methoxy-2,2-dimethylpropan-1-one
1-(5-{[(5-chlorothiophen-2-yl)methyl]sulfanyl}-3-(4-methyloxan-3-yl)-1H-1,2,4-triazol-1-
yl)-3-methoxy-2,2-dimethylpropan-1-one
1-(5-{[(5-chlorothiophen-2-yl)methyl]sulfanyl}-3-[3-methyl-1-(pyrrolidine-1-
sulfonyl)piperidin-4-yl]-1H-1,2,4-triazol-1-yl)-3-methoxy-2,2-dimethylpropan-1-one
1-(5-{[(5-chlorothiophen-2-yl)methyl]sulfanyl}-3-{1-[(3-hydroxypyrrolidin-1-
yl)sulfonyl]-2-methylpiperidin-3-yl}-1H-1,2,4-triazol-1-yl)-3-methoxy-2,2-
dimethylpropan-1-one
1-(5-{[4-(aminomethyl)phenyl]methoxy}-3-(2-methylpyrrolidin-3-yl)-1H-1,2,4-triazol-1-
yl)-3-methoxy-2,2-dimethylpropan-1-one
1-(5-{[4-(aminomethyl)phenyl]methoxy}-3-{3-methyl-1-[2-(morpholin-4-
yl)acetyl]azetidin-2-yl}-1H-1,2,4-triazol-1-yl)-3-methoxy-2,2-dimethylpropan-1-one
1-(dimethylcarbamoyl)-3-(5-{[(4-fluorophenyl)methyl](methyl)amino}-1-(thiophene-3-
carbonyl)-1H-1,2,4-triazol-3-yl)-4-methylpyrrolidine-2-carboxylic acid
1-({3-[5-({[4-(aminomethyl)phenyl]methyl}amino)-1-(furan-3-carbonyl)-1H-1,2,4-triazol-
3-yl]-2-(trifluoromethyl)piperazin-1-yl}sulfonyl)pyrrolidin-3-ol
1-[2-(5-{[(4-fluorophenyl)methyl]amino}-1-(5-methylfuran-3-carbonyl)-1H-1,2,4-triazol-
3-yl)-3-(trifluoromethyl)azetidin-1-yl]-2,2-dimethylpropan-1-one
1-[2-(5-{[(4-fluorophenyl)methyl]amino}-1-(furan-3-carbonyl)-1H-1,2,4-triazol-3-yl)-3-
(trifluoromethyl)piperazine-1-carbonyl]pyrrolidin-3-ol
1-[2-(5-{[(5-chlorothiophen-2-yl)methyl](methyl)amino}-1-(2-methoxybenzoyl)-1H-
1,2,4-triazol-3-yl)-3-(trifluoromethyl)azetidin-1-yl]-2,2-dimethylpropan-1-one
1-[2-(5-{[(5-chlorothiophen-2-yl)methyl]amino}-1-(furan-3-carbonyl)-1H-1,2,4-triazol-3-
yl)-3-(trifluoromethyl)piperazin-1-yl]-2-(morpholin-4-yl)ethan-1-one
1-[2-(5-{[4-(aminomethyl)phenyl]methoxy}-1-(2-chlorobenzoyl)-1H-1,2,4-triazol-3-
yl)azetidine-1-carbonyl]pyrrolidin-3-ol
1-[3-(5-{[(4-fluorophenyl)methyl](methyl)amino}-1-(2-methylfuran-3-carbonyl)-1H-
1,2,4-triazol-3-yl)pyrrolidin-1-yl]-2,2-dimethylpropan-1-one
1-[3-(5-{[(4-fluorophenyl)methyl]amino}-1-(furan-2-carbonyl)-1H-1,2,4-triazol-3-yl)-2-
(trifluoromethyl)piperazin-1-yl]-2,2-dimethylpropan-1-one
1-[3-(5-{[(4-fluorophenyl)methyl]amino}-1-(thiophene-3-carbonyl)-1H-1,2,4-triazol-3-
yl)-2-methylpyrrolidine-1-carbonyl]pyrrolidin-3-ol
1-[3-(5-{[(4-fluorophenyl)methyl]sulfanyl}-1-(1,3-thiazole-4-carbonyl)-1H-1,2,4-triazol-
3-yl)-2-methylazetidine-1-carbonyl]pyrrolidin-3-ol
1-[3-(5-{[(4-fluorophenyl)methyl]sulfanyl}-1-(thiophene-2-carbonyl)-1H-1,2,4-triazol-3-
yl)pyrrolidine-1-carbonyl]pyrrolidin-3-ol
1-[3-(5-{[(5-chlorothiophen-2-yl)methyl](methyl)amino}-1-(2-fluorobenzoyl)-1H-1,2,4-
triazol-3-yl)-2-(trifluoromethyl)piperazin-1-yl]-2,2-dimethylpropan-1-one
1-[3-(5-{[(5-chlorothiophen-2-yl)methyl](methyl)amino}-1H-1,2,4-triazol-3-yl)-2-
methylpyrrolidine-1-carbonyl]pyrrolidin-3-ol
1-[3-(5-{[(5-chlorothiophen-2-yl)methyl]amino}-1-(4-methylfuran-3-carbonyl)-1H-1,2,4-
triazol-3-yl)-2-methylazetidine-1-carbonyl]pyrrolidin-3-ol
1-[3-(5-{[4-(aminomethyl)phenyl]methoxy}-1-(4-methylfuran-3-carbonyl)-1H-1,2,4-
triazol-3-yl)-4-methylpyrrolidin-1-yl]-2-(morpholin-4-yl)ethan-1-one
1-[3-(5-{[4-(aminomethyl)phenyl]methoxy}-1-(thiophene-3-carbonyl)-1H-1,2,4-triazol-3-
yl)-2-methylazetidin-1-yl]-2,2-dimethylpropan-1-one
1-[4-(5-{[(4-fluorophenyl)methyl]sulfanyl}-1-(1,3-thiazole-4-carbonyl)-1H-1,2,4-triazol-
3-yl)-3-methylpiperidin-1-yl]-2-(morpholin-4-yl)ethan-1-one
1-[4-(5-{[(4-fluorophenyl)methyl]sulfanyl}-1-(thiophene-2-carbonyl)-1H-1,2,4-triazol-3-
yl)-2-hydroxypyrrolidin-1-yl]-2-(morpholin-4-yl)ethan-1-one
1-[4-(5-{[(5-chlorothiophen-2-yl)methyl]amino}-1-(4-methylfuran-3-carbonyl)-1H-1,2,4-
triazol-3-yl)-3-methylpiperidin-1-yl]-2-(morpholin-4-yl)ethan-1-one
1-[4-(5-{[4-(aminomethyl)phenyl]methoxy}-1-(4-methylfuran-3-carbonyl)-1H-1,2,4-
triazol-3-yl)-3-methylpiperidin-1-yl]-2,2-dimethylpropan-1-one
1-[5-({[4-(aminomethyl)phenyl]methyl}(methyl)amino)-3-[1-(pyrrolidine-1-carbonyl)-2-
(trifluoromethyl)pyrrolidin-3-yl]-1H-1,2,4-triazol-1-yl]-2,2-dimethylpropan-1-one
1-[5-({[4-(aminomethyl)phenyl]methyl}(methyl)amino)-3-[2-methyl-1-(pyrrolidine-1-
sulfonyl)pyrrolidin-3-yl]-1H-1,2,4-triazol-1-yl]-3-hydroxy-2,2-dimethylpropan-1-one
1-[5-({[4-(aminomethyl)phenyl]methyl}(methyl)amino)-3-{3-methyl-1-[2-(morpholin-4-
yl)acetyl]azetidin-2-yl}-1H-1,2,4-triazol-1-yl]-3-hydroxy-2,2-dimethylpropan-1-one
1-[5-({[4-(aminomethyl)phenyl]methyl}amino)-3-[3-methyl-1-(pyrrolidine-1-
sulfonyl)piperazin-2-yl]-1H-1,2,4-triazol-1-yl]-3-methoxy-2,2-dimethylpropan-1-one
1-[5-({[4-(aminomethyl)phenyl]methyl}amino)-3-{1-[(3-hydroxypyrrolidin-1-
yl)sulfonyl]-3-methylpiperazin-2-yl}-1H-1,2,4-triazol-1-yl]-3-methoxy-2,2-
dimethylpropan-1-one
1-[5-({[4-(aminomethyl)phenyl]methyl}amino)-3-{1-[(3-hydroxypyrrolidin-1-
yl)sulfonyl]-4-methylpyrrolidin-3-yl}-1H-1,2,4-triazol-1-yl]-3-methoxy-2,2-
dimethylpropan-1-one
1-[5-({[4-(aminomethyl)phenyl]methyl}sulfanyl)-3-(4-methyloxan-3-yl)-1H-1,2,4-triazol-
1-yl]-3-hydroxy-2,2-dimethylpropan-1-one
1-[5-({[4-(aminomethyl)phenyl]methyl}sulfanyl)-3-[1-(3-hydroxypyrrolidine-1-carbonyl)-
4-(trifluoromethyl)piperidin-3-yl]-1H-1,2,4-triazol-1-yl]-2,2-dimethylpropan-1-one
1-[5-({[4-(aminomethyl)phenyl]methyl}sulfanyl)-3-[3-methyl-1-(pyrrolidine-1-
sulfonyl)piperidin-4-yl]-1H-1,2,4-triazol-1-yl]-3-hydroxy-2,2-dimethylpropan-1-one
1-benzoyl-N-[(5-chlorothiophen-2-yl)methyl]-N-methyl-3-[1-(morpholine-4-
carbonyl)piperidin-3-yl]-1H-1,2,4-triazol-5-amine
1-{2-[5-({[4-(aminomethyl)phenyl]methyl}(methyl)amino)-1-benzoyl-1H-1,2,4-triazol-3-
yl]azetidine-1-carbonyl}pyrrolidin-3-ol
1-{2-[5-({[4-(aminomethyl)phenyl]methyl}sulfanyl)-1-(2-methoxybenzoyl)-1H-1,2,4-
triazol-3-yl]-3-(trifluoromethyl)piperazin-1-yl}-2,2-dimethylpropan-1-one
1-{3-[1-(2,2-dimethylpropanoyl)-4-methylpiperidin-3-yl]-5-{[(4-
fluorophenyl)methyl](methyl)amino}-1H-1,2,4-triazol-1-yl}-3-methoxy-2,2-
dimethylpropan-1-one
1-{3-[1-(2,2-dimethylpropanoyl)-5-[(4-fluorophenyl)methoxy]-1H-1,2,4-triazol-3-yl]-5-
hydroxy-2-(trifluoromethyl)pyrrolidin-1-yl}-2,2-dimethylpropan-1-one
1-{3-[1-(2-chlorobenzoyl)-5-{[(4-fluorophenyl)methyl](methyl)amino}-1H-1,2,4-triazol-
3-yl]piperazine-1-carbonyl}pyrrolidin-3-ol
1-{3-[1-(2-chlorobenzoyl)-5-{[(5-chlorothiophen-2-yl)methyl]sulfanyl}-1H-1,2,4-triazol-
3-yl]piperidin-1-yl}-2-(morpholin-4-yl)ethan-1-one
1-{3-[5-({[4-(aminomethyl)phenyl]methyl}amino)-1-(furan-3-carbonyl)-1H-1,2,4-triazol-
3-yl]-2-(trifluoromethyl)piperidin-1-yl}-2,2-dimethylpropan-1-one
1-{3-[5-({[4-(aminomethyl)phenyl]methyl}amino)-1-(furan-3-carbonyl)-1H-1,2,4-triazol-
3-yl]-5-hydroxy-2-(trifluoromethyl)pyrrolidin-1-yl}-2,2-dimethylpropan-1-one
1-{4-[5-({[4-(aminomethyl)phenyl]methyl}(methyl)amino)-1-(1,3-thiazole-4-carbonyl)-
1H-1,2,4-triazol-3-yl]-3-methylpiperidin-1-yl}-2,2-dimethylpropan-1-one
1-{4-[5-({[4-(aminomethyl)phenyl]methyl}sulfanyl)-1-(2-fluorobenzoyl)-1H-1,2,4-triazol-
3-yl]-3-(trifluoromethyl)piperidine-1-carbonyl}pyrrolidin-3-ol
1-{5-[(4-fluorophenyl)methoxy]-3-[1-methanesulfonyl-2-(trifluoromethyl)pyrrolidin-3-
yl]-1H-1,2,4-triazol-1-yl}-2,2-dimethylpropan-1-one
1-{5-[(4-fluorophenyl)methoxy]-3-[3-methyl-1-(pyrrolidine-1-sulfonyl)piperazin-2-yl]-
1H-1,2,4-triazol-1-yl}-3-hydroxy-2,2-dimethylpropan-1-one
1-{5-[(4-fluorophenyl)methoxy]-3-{1-[(3-hydroxypyrrolidin-1-yl)sulfonyl]-3-
methylpiperazin-2-yl}-1H-1,2,4-triazol-1-yl}-3-hydroxy-2,2-dimethylpropan-1-one
1-{5-[(4-fluorophenyl)methoxy]-3-{1-[(3-hydroxypyrrolidin-1-yl)sulfonyl]-4-
(trifluoromethyl)piperidin-3-yl}-1H-1,2,4-triazol-1-yl}-2,2-dimethylpropan-1-one
1-{5-[(4-fluorophenyl)methoxy]-3-{2-methyl-1-[2-(morpholin-4-yl)acetyl]azetidin-3-yl}-
1H-1,2,4-triazol-1-yl}-3-hydroxy-2,2-dimethylpropan-1-one
1-{5-[(4-fluorophenyl)methoxy]-3-{4-[(3-hydroxypyrrolidin-1-yl)sulfonyl]-3-
(trifluoromethyl)piperazin-2-yl}-1H-1,2,4-triazol-1-yl}-2,2-dimethylpropan-1-one
1-{5-[(5-chlorothiophen-2-yl)methoxy]-3-(4-methyloxolan-3-yl)-1H-1,2,4-triazol-1-yl}-3-
hydroxy-2,2-dimethylpropan-1-one
1-{5-[(5-chlorothiophen-2-yl)methoxy]-3-[1-(morpholine-4-carbonyl)-2-
(trifluoromethyl)pyrrolidin-3-yl]-1H-1,2,4-triazol-1-yl}-2,2-dimethylpropan-1-one
1-{5-[(5-chlorothiophen-2-yl)methoxy]-3-[5-hydroxy-1-(3-hydroxypyrrolidine-1-
carbonyl)-2-methylpyrrolidin-3-yl]-1H-1,2,4-triazol-1-yl}-3-hydroxy-2,2-dimethylpropan-
1-one
1-{[3-(5-{[(4-fluorophenyl)methyl]amino}-1-(5-methylfuran-3-carbonyl)-1H-1,2,4-
triazol-3-yl)-2-(trifluoromethyl)pyrrolidin-1-yl]sulfonyl}pyrrolidin-3-ol
1-{[3-(5-{[(4-fluorophenyl)methyl]sulfanyl}-1-(2-methoxybenzoyl)-1H-1,2,4-triazol-3-
yl)-2-(trifluoromethyl)pyrrolidin-1-yl]sulfonyl}pyrrolidin-3-ol
1-{[4-(5-{[(5-chlorothiophen-2-yl)methyl]sulfanyl}-1-(2-methylfuran-3-carbonyl)-
1H-1,2,4-triazol-3-yl)piperidin-1-yl]sulfonyl}pyrrolidin-3-ol
2-(1-benzoyl-5-{[(4-fluorophenyl)methyl]sulfanyl}-1H-1,2,4-triazol-3-yl)piperazine
2-(2-{1-benzoyl-5-[(4-fluorophenyl)methoxy]-1H-1,2,4-triazol-3-yl}azetidin-1-yl)-1-
(morpholin-4-yl)ethan-1-one
2-(3-{5-[(5-chlorothiophen-2-yl)methoxy]-1-(2-methoxybenzoyl)-1H-1,2,4-triazol-3-yl}-
2-(trifluoromethyl)pyrrolidin-1-yl)-1-(morpholin-4-yl)ethan-1-one
2-(5-{[(4-fluorophenyl)methyl]sulfanyl}-1-(2-methoxybenzoyl)-1H-1,2,4-triazol-3-yl)-1-
(pyrrolidine-1-carbonyl)-3-(trifluoromethyl)piperazine
2-(5-{[(5-chlorothiophen-2-yl)methyl]sulfanyl}-1-(4-methylfuran-3-carbonyl)-1H-1,2,4-
triazol-3-yl)-N,N,3-trimethylpiperazine-1-sulfonamide
2-(5-{[4-(aminomethyl)phenyl]methoxy}-1-(furan-3-carbonyl)-1H-1,2,4-triazol-3-yl)-
N,N-dimethyl-3-(trifluoromethyl)azetidine-1-carboxamide
2-(5-{[4-(aminomethyl)phenyl]methoxy}-1-(furan-3-carbonyl)-1H-1,2,4-triazol-3-yl)-
N,N-dimethyl-4-oxo-3-(trifluoromethyl)azetidine-1-sulfonamide
2-[1-(2-fluorobenzoyl)-5-[(4-fluorophenyl)methoxy]-1H-1,2,4-triazol-3-yl]-3-
(trifluoromethyl)piperazine
2-[3-(5-{[(4-fluorophenyl)methyl](methyl)amino}-1-(thiophene-3-carbonyl)-1H-1,2,4-
triazol-3-yl)-2-methylpiperazin-1-yl]-1-(morpholin-4-yl)ethan-1-one
2-[3-(5-{[(4-fluorophenyl)methyl]amino}-1-(thiophene-3-carbonyl)-1H-1,2,4-triazol-3-
yl)-4-methylpiperidin-1-yl]-1-(morpholin-4-yl)ethan-1-one
2-[3-(5-{[(4-fluorophenyl)methyl]sulfanyl}-1H-1,2,4-triazol-3-yl)-4-methylpiperidin-1-
yl]-1-(morpholin-4-yl)ethan-1-one
2-[3-(5-{[(5-chlorothiophen-2-yl)methyl](methyl)amino}-1H-1,2,4-triazol-3-yl)-2-
methylpiperazin-1-yl]-1-(morpholin-4-yl)ethan-1-one
2-[3-(5-{[(5-chlorothiophen-2-yl)methyl]sulfanyl}-1-(furan-2-carbonyl)-1H-1,2,4-triazol-
3-yl)-2-(trifluoromethyl)piperidin-1-yl]-1-(morpholin-4-yl)ethan-1-one
2-[4-(5-{[(4-fluorophenyl)methyl]amino}-1-(furan-2-carbonyl)-1H-1,2,4-triazol-3-yl)-3-
(trifluoromethyl)piperidin-1-yl]-1-(morpholin-4-yl)ethan-1-one
2-[4-(5-{[(5-chlorothiophen-2-yl)methyl](methyl)amino}-1-(2-fluorobenzoyl)-1H-1,2,4-
triazol-3-yl)-3-(trifluoromethyl)piperidin-1-yl]-1-(morpholin-4-yl)ethan-1-one
2-[4-(5-{[(5-chlorothiophen-2-yl)methyl]amino}-1-(2-methylfuran-3-carbonyl)-1H-1,2,4-
triazol-3-yl)-2-hydroxypyrrolidin-1-yl]-1-(morpholin-4-yl)ethan-1-one
2-[5-({[4-(aminomethyl)phenyl]methyl}(methyl)amino)-1-(2,2-dimethylpropanoyl)-1H-
1,2,4-triazol-3-yl]-N,N-dimethyl-4-oxo-3-(trifluoromethyl)azetidine-1-sulfonamide
2-[5-({[4-(aminomethyl)phenyl]methyl}amino)-1-(2-methylfuran-3-carbonyl)-1H-1,2,4-
triazol-3-yl]-N,N-dimethylpiperazine-1-carboxamide
2-[5-({[4-(aminomethyl)phenyl]methyl}amino)-1-(3-methoxy-2,2-dimethylpropanoyl)-
1H-1,2,4-triazol-3-yl]-N,N,3-trimethylazetidine-1-sulfonamide
2-[5-({[4-(aminomethyl)phenyl]methyl}amino)-1-(4-methylfuran-3-carbonyl)-1H-1,2,4-
triazol-3-yl]-N,N,3-trimethyl-4-oxoazetidine-1-carboxamide
2-chloro-3-(5-{[(4-fluorophenyl)methyl](methyl)amino}-3-[2-oxo-1-(pyrrolidine-1-
carbonyl)piperidin-3-yl]-1H-1,2,4-triazole-1-carbonyl)benzoic acid
2-chloro-3-(5-{[(4-fluorophenyl)methyl](methyl)amino}-3-{1-[2-(morpholin-4-yl)acetyl]-
2-oxopiperidin-3-yl}-1H-1,2,4-triazole-1-carbonyl)benzoic acid
2-chloro-3-(5-{[(4-fluorophenyl)methyl](methyl)amino}-3-{4-[2-(morpholin-4-
yl)acetyl]piperazin-2-yl}-1H-1,2,4-triazole-1-carbonyl)benzoic acid
2-chloro-3-(5-{[(4-fluorophenyl)methyl]amino}-3-[1-(3-hydroxypyrrolidine-1-
carbonyl)pyrrolidin-3-yl]-1H-1,2,4-triazole-1-carbonyl)benzoic acid
2-chloro-3-(5-{[(4-fluorophenyl)methyl]amino}-3-[1-(morpholine-4-carbonyl)pyrrolidin-
3-yl]-1H-1,2,4-triazole-1-carbonyl)benzoic acid
2-chloro-3-(5-{[(4-fluorophenyl)methyl]amino}-3-{1-[(3-hydroxypyrrolidin-1-
yl)sulfonyl]-4-oxoazetidin-2-yl}-1H-1,2,4-triazole-1-carbonyl)benzoic acid
2-chloro-3-(5-{[(5-chlorothiophen-2-yl)methyl]amino}-3-[1-(pyrrolidine-1-
sulfonyl)azetidin-3-yl]-1H-1,2,4-triazole-1-carbonyl)benzoic acid
2-chloro-3-(5-{[(5-chlorothiophen-2-yl)methyl]amino}-3-[1-(pyrrolidine-1-
sulfonyl)piperidin-3-yl]-1H-1,2,4-triazole-1-carbonyl)benzoic acid
2-chloro-3-(5-{[(5-chlorothiophen-2-yl)methyl]amino}-3-{1-[2-(morpholin-4-
yl)acetyl]pyrrolidin-3-yl}-1H-1,2,4-triazole-1-carbonyl)benzoic acid
2-chloro-3-(5-{[(5-chlorothiophen-2-yl)methyl]amino}-3-{5-hydroxy-1-[2-(morpholin-4-
yl)acetyl]pyrrolidin-3-yl}-1H-1,2,4-triazole-1-carbonyl)benzoic acid
2-chloro-3-(5-{[(5-chlorothiophen-2-yl)methyl]sulfanyl}-3-(1-methanesulfonyl-2-
oxopiperidin-3-yl)-1H-1,2,4-triazole-1-carbonyl)benzoic acid
2-chloro-3-(5-{[(5-chlorothiophen-2-yl)methyl]sulfanyl}-3-[1-(dimethylcarbamoyl)-4-
oxopyrrolidin-3-yl]-1H-1,2,4-triazole-1-carbonyl)benzoic acid
2-chloro-3-(5-{[(5-chlorothiophen-2-yl)methyl]sulfanyl}-3-[1-(morpholine-4-carbonyl)-4-
oxopyrrolidin-3-yl]-1H-1,2,4-triazole-1-carbonyl)benzoic acid
2-chloro-3-(5-{[(5-chlorothiophen-2-yl)methyl]sulfanyl}-3-{1-[(3-hydroxypyrrolidin-1-
yl)sulfonyl]-4-oxopyrrolidin-3-yl}-1H-1,2,4-triazole-1-carbonyl)benzoic acid
2-{2-[5-({[4-(aminomethyl)phenyl]methyl}amino)-1-(2-chlorobenzoyl)-1H-1,2,4-triazol-
3-yl]azetidin-1-yl}-1-(morpholin-4-yl)ethan-1-one
2-{2-[5-({[4-(aminomethyl)phenyl]methyl}amino)-1-(thiophene-3-carbonyl)-1H-1,2,4-
triazol-3-yl]-3-methylpiperazin-1-yl}-1-(morpholin-4-yl)ethan-1-one
2-{3-[5-({[4-(aminomethyl)phenyl]methyl}(methyl)amino)-1-(thiophene-2-carbonyl)-1H-
1,2,4-triazol-3-yl]azetidin-1-yl}-1-(morpholin-4-yl)ethan-1-one
2-{5-[(4-carbamimidoylphenyl)methoxy]-1-(1,3-thiazole-4-carbonyl)-1H-1,2,4-triazol-3-
yl}-N,N,3-trimethyl-4-oxoazetidine-1-carboxamide
2-{5-[(4-carbamimidoylphenyl)methoxy]-1-(2,2-dimethylpropanoyl)-1H-1,2,4-triazol-3-
yl}-N,N-dimethyl-3-(trifluoromethyl)azetidine-1-carboxamide
3-(1-benzoyl-5-{[(4-fluorophenyl)methyl]sulfanyl}-1H-1,2,4-triazol-3-yl)-1-(pyrrolidine-
1-carbonyl)piperidine
3-(1-benzoyl-5-{[(4-fluorophenyl)methyl]sulfanyl}-1H-1,2,4-triazol-3-yl)-1-(pyrrolidine-
1-sulfonyl)piperazine
3-(1-benzoyl-5-{[(4-fluorophenyl)methyl]sulfanyl}-1H-1,2,4-triazol-3-yl)-N,N-
dimethylpiperidine-1-sulfonamide
3-(5-{[(4-carbamimidoylphenyl)methyl](methyl)amino}-1-(2-chlorobenzoyl)-1H-1,2,4-
triazol-3-yl)-N,N-dimethyl-2-oxoazetidine-1-carboxamide
3-(5-{[(4-carbamimidoylphenyl)methyl](methyl)amino}-1-(3-methoxy-2,2-
dimethylpropanoyl)-1H-1,2,4-triazol-3-yl)-N,N,4-trimethyl-2-oxopyrrolidine-1-
carboxamide
3-(5-{[(4-carbamimidoylphenyl)methyl](methyl)amino}-1-(thiophene-3-carbonyl)-1H-
1,2,4-triazol-3-yl)-1-(3-hydroxypyrrolidine-1-carbonyl)-4-methylpiperidine-2-carboxylic
acid
3-(5-{[(4-carbamimidoylphenyl)methyl](methyl)amino}-3-[1-(dimethylsulfamoyl)-2-
oxopyrrolidin-3-yl]-1H-1,2,4-triazole-1-carbonyl)-2-chlorobenzoic acid
3-(5-{[(4-carbamimidoylphenyl)methyl](methyl)amino}-3-{1-[2-(morpholin-4-yl)-2-
oxoethyl]azetidin-3-yl}-1H-1,2,4-triazole-1-carbonyl)-2-chlorobenzoic acid
3-(5-{[(4-carbamimidoylphenyl)methyl](methyl)amino}-3-{1-[2-(morpholin-4-yl)acetyl]-
6-oxopiperidin-3-yl}-1H-1,2,4-triazole-1-carbonyl)-2-chlorobenzoic acid
3-(5-{[(4-carbamimidoylphenyl)methyl]amino}-1-(3-carboxybenzoyl)-1H-1,2,4-triazol-3-
yl)-1-[2-(morpholin-4-yl)-2-oxoethyl]pyrrolidine-2-carboxylic acid
3-(5-{[(4-carbamimidoylphenyl)methyl]amino}-3-{1-[2-(morpholin-4-yl)acetyl]-5-
oxopyrrolidin-3-yl}-1H-1,2,4-triazole-1-carbonyl)benzoic acid
3-(5-{[(4-carbamimidoylphenyl)methyl]amino}-3-{5-hydroxy-1-[2-(morpholin-4-yl)-2-
oxoethyl]pyrrolidin-3-yl}-1H-1,2,4-triazole-1-carbonyl)benzoic acid
3-(5-{[(4-carbamimidoylphenyl)methyl]sulfanyl}-1-(2-chlorobenzoyl)-1H-1,2,4-triazol-3-
yl)-1-[2-(morpholin-4-yl)-2-oxoethyl]piperidine-2-carboxylic acid
3-(5-{[(4-carbamimidoylphenyl)methyl]sulfanyl}-1-(2-chlorobenzoyl)-1H-1,2,4-triazol-3-
yl)-N,N-dimethylpiperazine-1-carboxamide
3-(5-{[(4-carbamimidoylphenyl)methyl]sulfanyl}-1-(2-methylfuran-3-carbonyl)-1H-1,2,4-
triazol-3-yl)-1-(pyrrolidine-1-carbonyl)piperidine-2-carboxylic acid
3-(5-{[(4-carbamimidoylphenyl)methyl]sulfanyl}-1-(3-carboxy-2-chlorobenzoyl)-1H-
1,2,4-triazol-3-yl)-1-(pyrrolidine-1-carbonyl)pyrrolidine-2-carboxylic acid
3-(5-{[(4-carbamimidoylphenyl)methyl]sulfanyl}-1-(3-methoxy-2,2-dimethylpropanoyl)-
1H-1,2,4-triazol-3-yl)-4-methylpyrrolidine-2-carboxylic acid
3-(5-{[(4-carbamimidoylphenyl)methyl]sulfanyl}-1-(4-methylfuran-3-carbonyl)-1H-1,2,4-
triazol-3-yl)-1-methanesulfonyl-4-methylpyrrolidine-2-carboxylic acid
3-(5-{[(4-carbamimidoylphenyl)methyl]sulfanyl}-1-(thiophene-3-carbonyl)-1H-1,2,4-
triazol-3-yl)-N,N,4-trimethylpiperidine-1-carboxamide
3-(5-{[(4-carbamimidoylphenyl)methyl]sulfanyl}-3-(piperidin-3-yl)-1H-1,2,4-triazole-1-
carbonyl)-2-chlorobenzoic acid
3-(5-{[(4-carbamimidoylphenyl)methyl]sulfanyl}-3-[3-oxo-1-(pyrrolidine-1-
sulfonyl)piperidin-4-yl]-1H-1,2,4-triazole-1-carbonyl)-2-chlorobenzoic acid
3-(5-{[(4-fluorophenyl)methyl](methyl)amino}-1-(2-methylfuran-3-carbonyl)-1H-1,2,4-
triazol-3-yl)-1-(morpholine-4-carbonyl)piperidine-2-carboxylic acid
3-(5-{[(4-fluorophenyl)methyl](methyl)amino}-1-(4-methylfuran-3-carbonyl)-1H-1,2,4-
triazol-3-yl)-1-(3-hydroxypyrrolidine-1-carbonyl)-4-methylpyrrolidine-2-carboxylic acid
3-(5-{[(4-fluorophenyl)methyl](methyl)amino}-1-(4-methylfuran-3-carbonyl)-1H-1,2,4-
triazol-3-yl)-4-methyl-1-(pyrrolidine-1-sulfonyl)piperidin-2-one
3-(5-{[(4-fluorophenyl)methyl](methyl)amino}-1-(5-methylfuran-3-carbonyl)-1H-1,2,4-
triazol-3-yl)-1-[(3-hydroxypyrrolidin-1-yl)sulfonyl]-4-(trifluoromethyl)azetidin-2-one
3-(5-{[(4-fluorophenyl)methyl](methyl)amino}-1-(5-methylfuran-3-carbonyl)-1H-1,2,4-
triazol-3-yl)-N,N-dimethyl-2-(trifluoromethyl)piperidine-1-carboxamide
3-(5-{[(4-fluorophenyl)methyl](methyl)amino}-1-(furan-2-carbonyl)-1H-1,2,4-triazol-3-
yl)-1-(pyrrolidine-1-sulfonyl)-4-(trifluoromethyl)azetidin-2-one
3-(5-{[(4-fluorophenyl)methyl](methyl)amino}-1-(furan-2-carbonyl)-1H-1,2,4-triazol-3-
yl)-N,N-dimethyl-4-(trifluoromethyl)pyrrolidine-1-carboxamide
3-(5-{[(4-fluorophenyl)methyl](methyl)amino}-1-(furan-3-carbonyl)-1H-1,2,4-triazol-3-
yl)-1-[2-(morpholin-4-yl)acetyl]-4-(trifluoromethyl)pyrrolidine-2-carboxylic acid
3-(5-{[(4-fluorophenyl)methyl](methyl)amino}-1-(furan-3-carbonyl)-1H-1,2,4-triazol-3-
yl)-4-(trifluoromethyl)azetidin-2-one
3-(5-{[(4-fluorophenyl)methyl]amino}-1-(2-methylfuran-3-carbonyl)-1H-1,2,4-triazol-3-
yl)-1-[(3-hydroxypyrrolidin-1-yl)sulfonyl]piperidin-2-one
3-(5-{[(4-fluorophenyl)methyl]amino}-1-(3-methoxy-2,2-dimethylpropanoyl)-1H-1,2,4-
triazol-3-yl)-4-methyl-1-(morpholine-4-carbonyl)pyrrolidin-2-one
3-(5-{[(4-fluorophenyl)methyl]amino}-1-(4-methylfuran-3-carbonyl)-1H-1,2,4-triazol-3-
yl)-1-[(3-hydroxypyrrolidin-1-yl)sulfonyl]-4-methylpyrrolidine-2-carboxylic acid
3-(5-{[(4-fluorophenyl)methyl]amino}-1-(furan-2-carbonyl)-1H-1,2,4-triazol-3-yl)-N,N-
dimethyl-2-(trifluoromethyl)azetidine-1-carboxamide
3-(5-{[(4-fluorophenyl)methyl]sulfanyl}-1-(1,3-thiazole-4-carbonyl)-1H-1,2,4-triazol-3-
yl)-1-[(3-hydroxypyrrolidin-1-yl)sulfonyl]-4-methylpyrrolidine-2-carboxylic acid
3-(5-{[(4-fluorophenyl)methyl]sulfanyl}-1-(3-hydroxy-2,2-dimethylpropanoyl)-1H-1,2,4-
triazol-3-yl)-4-methyl-1-[2-(morpholin-4-yl)-2-oxoethyl]azetidin-2-one
3-(5-{[(4-fluorophenyl)methyl]sulfanyl}-3-(oxan-3-yl)-1H-1,2,4-triazole-1-
carbonyl)benzoic acid
3-(5-{[(4-fluorophenyl)methyl]sulfanyl}-3-[6-oxo-1-(pyrrolidine-1-carbonyl)piperidin-3-
yl]-1H-1,2,4-triazole-1-carbonyl)benzoic acid
3-(5-{[(4-fluorophenyl)methyl]sulfanyl}-3-{1-[(3-hydroxypyrrolidin-1-yl)sulfonyl]-2-
oxopiperidin-3-yl}-1H-1,2,4-triazole-1-carbonyl)benzoic acid
3-(5-{[(5-chlorothiophen-2-yl)methyl](methyl)amino}-1-(1,3-thiazole-4-carbonyl)-1H-
1,2,4-triazol-3-yl)-1-(3-hydroxypyrrolidine-1-carbonyl)-4-methylpyrrolidine-2-carboxylic
acid
3-(5-{[(5-chlorothiophen-2-yl)methyl](methyl)amino}-1-(1,3-thiazole-4-carbonyl)-1H-
1,2,4-triazol-3-yl)-4-methyl-1-(pyrrolidine-1-sulfonyl)pyrrolidin-2-one
3-(5-{[(5-chlorothiophen-2-yl)methyl](methyl)amino}-1-(2-fluorobenzoyl)-1H-1,2,4-
triazol-3-yl)-N,N-dimethyl-4-(trifluoromethyl)pyrrolidine-1-carboxamide
3-(5-{[(5-chlorothiophen-2-yl)methyl](methyl)amino}-1-(3-hydroxy-2,2-
dimethylpropanoyl)-1H-1,2,4-triazol-3-yl)-4-methyl-1-(morpholine-4-carbonyl)pyrrolidin-
2-one
3-(5-{[(5-chlorothiophen-2-yl)methyl](methyl)amino}-1-(thiophene-2-carbonyl)-1H-1,2,4-
triazol-3-yl)-1-(pyrrolidine-1-carbonyl)piperidin-2-one
3-(5-{[(5-chlorothiophen-2-yl)methyl](methyl)amino}-1-(thiophene-2-carbonyl)-1H-1,2,4-
triazol-3-yl)-1-[(3-hydroxypyrrolidin-1-yl)sulfonyl]piperidine-2-carboxylic acid
3-(5-{[(5-chlorothiophen-2-yl)methyl](methyl)amino}-1H-1,2,4-triazol-3-yl)-1-
(dimethylcarbamoyl)-4-methylpyrrolidine-2-carboxylic acid
3-(5-{[(5-chlorothiophen-2-yl)methyl](methyl)amino}-3-(5-hydroxy-1-
methanesulfonylpyrrolidin-3-yl)-1H-1,2,4-triazole-1-carbonyl)benzoic acid
3-(5-{[(5-chlorothiophen-2-yl)methyl](methyl)amino}-3-[1-(2,2-
dimethylpropanoyl)pyrrolidin-3-yl]-1H-1,2,4-triazole-1-carbonyl)benzoic acid
3-(5-{[(5-chlorothiophen-2-yl)methyl](methyl)amino}-3-[1-(dimethylcarbamoyl)-2-
oxopiperidin-4-yl]-1H-1,2,4-triazole-1-carbonyl)benzoic acid
3-(5-{[(5-chlorothiophen-2-yl)methyl]amino}-1-(3-methoxy-2,2-dimethylpropanoyl)-1H-
1,2,4-triazol-3-yl)-1-(3-hydroxypyrrolidine-1-carbonyl)-4-methylpyrrolidin-2-one
3-(5-{[(5-chlorothiophen-2-yl)methyl]amino}-1-(3-methoxy-2,2-dimethylpropanoyl)-1H-
1,2,4-triazol-3-yl)-4-methyl-1-[2-(morpholin-4-yl)-2-oxoethyl]azetidin-2-one
3-(5-{[(5-chlorothiophen-2-yl)methyl]amino}-1-(4-methylfuran-3-carbonyl)-1H-1,2,4-
triazol-3-yl)-4-methyl-1-[2-(morpholin-4-yl)-2-oxoethyl]piperidin-2-one
3-(5-{[(5-chlorothiophen-2-yl)methyl]amino}-1-(furan-2-carbonyl)-1H-1,2,4-triazol-3-yl)-
N,N-dimethyl-2-oxo-4-(trifluoromethyl)piperidine-1-carboxamide
3-(5-{[(5-chlorothiophen-2-yl)methyl]sulfanyl}-1-(2-methylfuran-3-carbonyl)-1H-1,2,4-
triazol-3-yl)-1-[2-(morpholin-4-yl)-2-oxoethyl]pyrrolidine-2-carboxylic acid
3-(5-{[(5-chlorothiophen-2-yl)methyl]sulfanyl}-1-(3-methoxy-2,2-dimethylpropanoyl)-
1H-1,2,4-triazol-3-yl)-N,N,4-trimethylpiperidine-1-sulfonamide
3-(5-{[(5-chlorothiophen-2-yl)methyl]sulfanyl}-1-(4-methylfuran-3-carbonyl)-1H-1,2,4-
triazol-3-yl)-4-methylpiperidine-2-carboxylic acid
3-(5-{[(5-chlorothiophen-2-yl)methyl]sulfanyl}-1-(5-methylfuran-3-carbonyl)-1H-1,2,4-
triazol-3-yl)-1-(pyrrolidine-1-carbonyl)-4-(trifluoromethyl)piperidine
3-(5-{[(5-chlorothiophen-2-yl)methyl]sulfanyl}-1-(5-methylfuran-3-carbonyl)-1H-1,2,4-
triazol-3-yl)-4-(trifluoromethyl)piperidin-2-one
3-(5-{[(5-chlorothiophen-2-yl)methyl]sulfanyl}-1-(furan-3-carbonyl)-1H-1,2,4-triazol-3-
yl)-1-methanesulfonyl-4-(trifluoromethyl)piperidine-2-carboxylic acid
3-(5-{[(5-chlorothiophen-2-yl)methyl]sulfanyl}-1-(furan-3-carbonyl)-1H-1,2,4-triazol-3-
yl)-N,N-dimethyl-2-(trifluoromethyl)piperazine-1-sulfonamide
3-(5-{[(5-chlorothiophen-2-yl)methyl]sulfanyl}-1-(thiophene-3-carbonyl)-1H-1,2,4-
triazol-3-yl)-1-(2,2-dimethylpropanoyl)-4-methylpyrrolidin-2-one
3-(5-{[4-(aminomethyl)phenyl]methoxy}-1-(2-methylfuran-3-carbonyl)-1H-1,2,4-triazol-
3-yl)-1-[2-(morpholin-4-yl)-2-oxoethyl]pyrrolidin-2-one
3-(5-{[4-(aminomethyl)phenyl]methoxy}-1-(2-methylfuran-3-carbonyl)-1H-1,2,4-triazol-
3-yl)-N,N-dimethyl-4-oxopyrrolidine-1-sulfonamide
3-(5-{[4-(aminomethyl)phenyl]methoxy}-1-(3-methoxy-2,2-dimethylpropanoyl)-1H-
1,2,4-triazol-3-yl)-4-methyl-1-(morpholine-4-carbonyl)pyrrolidine-2-carboxylic acid
3-(5-{[4-(aminomethyl)phenyl]methoxy}-1-(4-methylfuran-3-carbonyl)-1H-1,2,4-triazol-
3-yl)-5-hydroxy-N,N,2-trimethylpyrrolidine-1-carboxamide
3-(5-{[4-(aminomethyl)phenyl]methoxy}-1-(5-methylfuran-3-carbonyl)-1H-1,2,4-triazol-
3-yl)-1-(morpholine-4-carbonyl)-4-(trifluoromethyl)piperidin-2-one
3-(5-{[4-(aminomethyl)phenyl]methoxy}-1-(5-methylfuran-3-carbonyl)-1H-1,2,4-triazol-
3-yl)-1-[2-(morpholin-4-yl)acetyl]-4-(trifluoromethyl)pyrrolidin-2-one
3-(5-{[4-(aminomethyl)phenyl]methoxy}-1-(furan-2-carbonyl)-1H-1,2,4-triazol-3-yl)-1-
(pyrrolidine-1-sulfonyl)-4-(trifluoromethyl)piperidine-2-carboxylic acid
3-(5-{[4-(aminomethyl)phenyl]methoxy}-1-(thiophene-3-carbonyl)-1H-1,2,4-triazol-3-yl)-
1-(dimethylsulfamoyl)-4-methylpyrrolidine-2-carboxylic acid
3-(5-{[4-(aminomethyl)phenyl]methoxy}-3-[1-(2,2-dimethylpropanoyl)-6-oxopiperidin-3-
yl]-1H-1,2,4-triazole-1-carbonyl)-2-chlorobenzoic acid
3-(5-{[4-(aminomethyl)phenyl]methoxy}-3-[5-oxo-1-(pyrrolidine-1-carbonyl)pyrrolidin-
3-yl]-1H-1,2,4-triazole-1-carbonyl)-2-chlorobenzoic acid
3-(5-{[4-(aminomethyl)phenyl]methoxy}-3-{1-[2-(morpholin-4-yl)-2-oxoethyl]-6-
oxopiperidin-3-yl}-1H-1,2,4-triazole-1-carbonyl)-2-chlorobenzoic acid
3-(azetidin-3-yl)-5-{[(5-chlorothiophen-2-yl)methyl]sulfanyl}-1-(2-methylfuran-3-
carbonyl)-1H-1,2,4-triazole
3-[1-(2-chlorobenzoyl)-5-{[(4-fluorophenyl)methyl]amino}-1H-1,2,4-triazol-3-yl]-N,N-
dimethylpiperidine-1-sulfonamide
3-[1-(2-chlorobenzoyl)-5-{[(5-chlorothiophen-2-yl)methyl]amino}-1H-1,2,4-triazol-3-yl]-
1-(pyrrolidine-1-carbonyl)azetidin-2-one
3-[1-(2-chlorobenzoyl)-5-{[(5-chlorothiophen-2-yl)methyl]sulfanyl}-1H-1,2,4-triazol-3-
yl]-1-(dimethylsulfamoyl)piperidine-2-carboxylic acid
3-[1-(2-fluorobenzoyl)-5-[(4-fluorophenyl)methoxy]-1H-1,2,4-triazol-3-yl]-N,N-dimethyl-
2-(trifluoromethyl)pyrrolidine-1-carboxamide
3-[1-(2-fluorobenzoyl)-5-{[(4-fluorophenyl)methyl]sulfanyl}-1H-1,2,4-triazol-3-yl]-N,N-
dimethyl-2-(trifluoromethyl)azetidine-1-carboxamide
3-[1-(3-carboxy-2-chlorobenzoyl)-5-{[(4-fluorophenyl)methyl](methyl)amino}-1H-1,2,4-
triazol-3-yl]-1-[(3-hydroxypyrrolidin-1-yl)sulfonyl]piperidine-2-carboxylic acid
3-[1-(3-carboxybenzoyl)-5-{[(5-chlorothiophen-2-yl)methyl](methyl)amino}-1H-1,2,4-
triazol-3-yl]-1-(morpholine-4-carbonyl)piperidine-2-carboxylic acid
3-[5-({[4-(aminomethyl)phenyl]methyl}(methyl)amino)-1-(2-fluorobenzoyl)-1H-1,2,4-
triazol-3-yl]-1-(pyrrolidine-1-sulfonyl)-4-(trifluoromethyl)piperidine-2-carboxylic acid
3-[5-({[4-(aminomethyl)phenyl]methyl}(methyl)amino)-1-(2-methoxybenzoyl)-1H-1,2,4-
triazol-3-yl]-1-(morpholine-4-carbonyl)-4-(trifluoromethyl)piperidin-2-one
3-[5-({[4-(aminomethyl)phenyl]methyl}(methyl)amino)-1-(2-methoxybenzoyl)-1H-1,2,4-
triazol-3-yl]-1-(pyrrolidine-1-carbonyl)-4-(trifluoromethyl)pyrrolidin-2-one
3-[5-({[4-(aminomethyl)phenyl]methyl}(methyl)amino)-1-(2-methoxybenzoyl)-1H-1,2,4-
triazol-3-yl]-1-[2-(morpholin-4-yl)acetyl]-4-(trifluoromethyl)azetidin-2-one
3-[5-({[4-(aminomethyl)phenyl]methyl}(methyl)amino)-1-(2-methoxybenzoyl)-1H-1,2,4-
triazol-3-yl]-1-[2-(morpholin-4-yl)acetyl]-4-(trifluoromethyl)pyrrolidin-2-one
3-[5-({[4-(aminomethyl)phenyl]methyl}(methyl)amino)-1-benzoyl-1H-1,2,4-triazol-3-yl]-
N,N-dimethyl-2-oxoazetidine-1-carboxamide
3-[5-({[4-(aminomethyl)phenyl]methyl}(methyl)amino)-1H-1,2,4-triazol-3-yl]-1-(3-
hydroxypyrrolidine-1-carbonyl)-4-methylpiperidine-2-carboxylic acid
3-[5-({[4-(aminomethyl)phenyl]methyl}(methyl)amino)-1H-1,2,4-triazol-3-yl]-1-[(3-
hydroxypyrrolidin-1-yl)sulfonyl]-4-methylpyrrolidin-2-one
3-[5-({[4-(aminomethyl)phenyl]methyl}(methyl)amino)-3-[1-(dimethylsulfamoyl)-2-
oxoazetidin-3-yl]-1H-1,2,4-triazole-1-carbonyl]benzoic acid
3-[5-({[4-(aminomethyl)phenyl]methyl}(methyl)amino)-3-[1-(dimethylsulfamoyl)-4-
oxopyrrolidin-3-yl]-1H-1,2,4-triazole-1-carbonyl]benzoic acid
3-[5-({[4-(aminomethyl)phenyl]methyl}(methyl)amino)-3-{1-[2-(morpholin-4-yl)-2-
oxoethyl]-2-oxopyrrolidin-3-yl}-1H-1,2,4-triazole-1-carbonyl]benzoic acid
3-[5-({[4-(aminomethyl)phenyl]methyl}(methyl)amino)-3-{1-[2-(morpholin-4-yl)-2-
oxoethyl]pyrrolidin-3-yl}-1H-1,2,4-triazole-1-carbonyl]benzoic acid
3-[5-({[4-(aminomethyl)phenyl]methyl}amino)-1-(2-chlorobenzoyl)-1H-1,2,4-triazol-3-
yl]-1-(2,2-dimethylpropanoyl)azetidin-2-one
3-[5-({[4-(aminomethyl)phenyl]methyl}amino)-1-(2-chlorobenzoyl)-1H-1,2,4-triazol-3-
yl]-1-methanesulfonylazetidin-2-one
3-[5-({[4-(aminomethyl)phenyl]methyl}amino)-1-(3-carboxy-2-chlorobenzoyl)-1H-1,2,4-
triazol-3-yl]-1-(dimethylcarbamoyl)piperidine-2-carboxylic acid
3-[5-({[4-(aminomethyl)phenyl]methyl}amino)-1-(3-carboxy-2-chlorobenzoyl)-1H-1,2,4-
triazol-3-yl]-1-[2-(morpholin-4-yl)acetyl]piperidine-2-carboxylic acid
3-[5-({[4-(aminomethyl)phenyl]methyl}amino)-1-(4-methylfuran-3-carbonyl)-1H-1,2,4-
triazol-3-yl]-N,N,4-trimethyl-2-oxopiperidine-1-sulfonamide
3-[5-({[4-(aminomethyl)phenyl]methyl}amino)-1-(5-methylfuran-3-carbonyl)-1H-1,2,4-
triazol-3-yl]-1-(2,2-dimethylpropanoyl)-4-(trifluoromethyl)pyrrolidine-2-carboxylic acid
3-[5-({[4-(aminomethyl)phenyl]methyl}amino)-1-(5-methylfuran-3-carbonyl)-1H-1,2,4-
triazol-3-yl]-1-(pyrrolidine-1-sulfonyl)-4-(trifluoromethyl)pyrrolidine-2-carboxylic acid
3-[5-({[4-(aminomethyl)phenyl]methyl}amino)-1-(5-methylfuran-3-carbonyl)-1H-1,2,4-
triazol-3-yl]-N,N-dimethyl-2-(trifluoromethyl)pyrrolidine-1-sulfonamide
3-[5-({[4-(aminomethyl)phenyl]methyl}amino)-1-(furan-2-carbonyl)-1H-1,2,4-triazol-3-
yl]-5-hydroxy-N,N-dimethyl-2-(trifluoromethyl)pyrrolidine-1-sulfonamide
3-[5-({[4-(aminomethyl)phenyl]methyl}amino)-3-(1-methanesulfonylpiperidin-3-yl)-1H-
1,2,4-triazole-1-carbonyl]-2-chlorobenzoic acid
3-[5-({[4-(aminomethyl)phenyl]methyl}amino)-3-[1-(morpholine-4-carbonyl)-4-
oxoazetidin-2-yl]-1H-1,2,4-triazole-1-carbonyl]-2-chlorobenzoic acid
3-[5-({[4-(aminomethyl)phenyl]methyl}sulfanyl)-1-(1,3-thiazole-4-carbonyl)-1H-1,2,4-
triazol-3-yl]-4-methylpiperidine-2-carboxylic acid
3-[5-({[4-(aminomethyl)phenyl]methyl}sulfanyl)-1-(2,2-dimethylpropanoyl)-1H-1,2,4-
triazol-3-yl]-1-(morpholine-4-carbonyl)-4-(trifluoromethyl)azetidin-2-one
3-[5-({[4-(aminomethyl)phenyl]methyl}sulfanyl)-1-(2,2-dimethylpropanoyl)-1H-1,2,4-
triazol-3-yl]-1-methanesulfonyl-4-(trifluoromethyl)piperidine-2-carboxylic acid
3-[5-({[4-(aminomethyl)phenyl]methyl}sulfanyl)-1-(2-methoxybenzoyl)-1H-1,2,4-triazol-
3-yl]-4-(trifluoromethyl)piperidin-2-one
3-[5-({[4-(aminomethyl)phenyl]methyl}sulfanyl)-1-(3-carboxybenzoyl)-1H-1,2,4-triazol-
3-yl]-1-(pyrrolidine-1-carbonyl)piperidine-2-carboxylic acid
3-[5-({[4-(aminomethyl)phenyl]methyl}sulfanyl)-1-(3-hydroxy-2,2-dimethylpropanoyl)-
1H-1,2,4-triazol-3-yl]-4-methylpyrrolidin-2-one
3-[5-({[4-(aminomethyl)phenyl]methyl}sulfanyl)-1-(thiophene-2-carbonyl)-1H-1,2,4-
triazol-3-yl]-1-methanesulfonylpiperidin-2-one
3-[5-({[4-(aminomethyl)phenyl]methyl}sulfanyl)-1-(thiophene-2-carbonyl)-1H-1,2,4-
triazol-3-yl]-N,N-dimethyl-4-oxopyrrolidine-1-carboxamide
3-[5-({[4-(aminomethyl)phenyl]methyl}sulfanyl)-1-benzoyl-1H-1,2,4-triazol-3-yl]-1-
(dimethylsulfamoyl)piperidine-2-carboxylic acid
3-[5-({[4-(aminomethyl)phenyl]methyl}sulfanyl)-1-benzoyl-1H-1,2,4-triazol-3-yl]-N,N-
dimethylpiperazine-1-carboxamide
3-[5-({[4-(aminomethyl)phenyl]methyl}sulfanyl)-1H-1,2,4-triazol-3-yl]-1-(2,2-
dimethylpropanoyl)-4-methylpyrrolidin-2-one
3-[5-({[4-(aminomethyl)phenyl]methyl}sulfanyl)-1H-1,2,4-triazol-3-yl]-N,N,4-
trimethylpiperidine-1-carboxamide
3-[5-({[4-(aminomethyl)phenyl]methyl}sulfanyl)-3-(azetidin-3-yl)-1H-1,2,4-triazole-1-
carbonyl]benzoic acid
3-[5-({[4-(aminomethyl)phenyl]methyl}sulfanyl)-3-[1-(morpholine-4-carbonyl)azetidin-2-
yl]-1H-1,2,4-triazole-1-carbonyl]benzoic acid
3-[5-({[4-(aminomethyl)phenyl]methyl}sulfanyl)-3-{1-[(3-hydroxypyrrolidin-1-
yl)sulfonyl]piperidin-4-yl}-1H-1,2,4-triazole-1-carbonyl]benzoic acid
3-{1-benzoyl-5-[(4-fluorophenyl)methoxy]-1H-1,2,4-triazol-3-yl}-1-[2-(morpholin-4-yl)-
2-oxoethyl]piperidine-2-carboxylic acid
3-{1-benzoyl-5-[(4-fluorophenyl)methoxy]-1H-1,2,4-triazol-3-yl}-1-
methanesulfonylazetidin-2-one
3-{1-benzoyl-5-[(5-chlorothiophen-2-yl)methoxy]-1H-1,2,4-triazol-3-yl}-N,N-
dimethylpyrrolidine-1-sulfonamide
3-{3-[1-(2,2-dimethylpropanoyl)-4-oxopyrrolidin-3-yl]-5-[(4-fluorophenyl)methoxy]-1H-
1,2,4-triazole-1-carbonyl}benzoic acid
3-{3-[1-(dimethylcarbamoyl)piperazin-2-yl]-5-[(4-fluorophenyl)methoxy]-1H-1,2,4-
triazole-1-carbonyl}benzoic acid
3-{5-[(4-carbamimidoylphenyl)methoxy]-1-(1,3-thiazole-4-carbonyl)-1H-1,2,4-triazol-3-
yl}-5-hydroxy-N,N,2-trimethylpyrrolidine-1-carboxamide
3-{5-[(4-carbamimidoylphenyl)methoxy]-1-(2-methoxybenzoyl)-1H-1,2,4-triazol-3-yl}-1-
(2,2-dimethylpropanoyl)-4-(trifluoromethyl)pyrrolidine-2-carboxylic acid
3-{5-[(4-carbamimidoylphenyl)methoxy]-1-(3-hydroxy-2,2-dimethylpropanoyl)-1H-1,2,4-
triazol-3-yl}-4-methyl-1-(morpholine-4-carbonyl)pyrrolidine-2-carboxylic acid
3-{5-[(4-carbamimidoylphenyl)methoxy]-1-(thiophene-2-carbonyl)-1H-1,2,4-triazol-3-
yl}-1-(dimethylcarbamoyl)piperidine-2-carboxylic acid
3-{5-[(4-carbamimidoylphenyl)methoxy]-1H-1,2,4-triazol-3-yl}-1-(dimethylsulfamoyl)-4-
methylpyrrolidine-2-carboxylic acid
3-{5-[(4-carbamimidoylphenyl)methoxy]-3-(5-hydroxypyrrolidin-3-yl)-1H-1,2,4-triazole-
1-carbonyl}benzoic acid
3-{5-[(4-carbamimidoylphenyl)methoxy]-3-[1-(3-hydroxypyrrolidine-1-carbonyl)-5-
oxopyrrolidin-3-yl]-1H-1,2,4-triazole-1-carbonyl}benzoic acid
3-{5-[(4-carbamimidoylphenyl)methoxy]-3-[1-(morpholine-4-carbonyl)-3-oxopiperidin-4-
yl]-1H-1,2,4-triazole-1-carbonyl}benzoic acid
3-{5-[(4-carbamimidoylphenyl)methoxy]-3-{1-[2-(morpholin-4-yl)-2-oxoethyl]-2-
oxopiperidin-4-yl}-1H-1,2,4-triazole-1-carbonyl}benzoic acid
3-{5-[(4-fluorophenyl)methoxy]-1-(1,3-thiazole-4-carbonyl)-1H-1,2,4-triazol-3-yl}-1-
methanesulfonyl-4-methylpyrrolidine-2-carboxylic acid
3-{5-[(4-fluorophenyl)methoxy]-1-(1,3-thiazole-4-carbonyl)-1H-1,2,4-triazol-3-yl}-4-
methylpiperidine
3-{5-[(4-fluorophenyl)methoxy]-1-(2-methoxybenzoyl)-1H-1,2,4-triazol-3-yl}-1-
(pyrrolidine-1-sulfonyl)-4-(trifluoromethyl)pyrrolidine-2-carboxylic acid
3-{5-[(4-fluorophenyl)methoxy]-1-(2-methoxybenzoyl)-1H-1,2,4-triazol-3-yl}-N,N-
dimethyl-2-(trifluoromethyl)pyrrolidine-1-sulfonamide
3-{5-[(4-fluorophenyl)methoxy]-1-(thiophene-2-carbonyl)-1H-1,2,4-triazol-3-yl}-1-[2-
(morpholin-4-yl)acetyl]piperidine-2-carboxylic acid
3-{5-[(4-fluorophenyl)methoxy]-1-(thiophene-2-carbonyl)-1H-1,2,4-triazol-3-yl}-1-
methanesulfonylpiperidine
3-{5-[(4-fluorophenyl)methoxy]-1-(thiophene-2-carbonyl)-1H-1,2,4-triazol-3-
yl}piperidine
3-{5-[(4-fluorophenyl)methoxy]-1H-1,2,4-triazol-3-yl}-1-(3-hydroxypyrrolidine-1-
carbonyl)-4-methylpiperidin-2-one
3-{5-[(4-fluorophenyl)methoxy]-3-(1-methanesulfonylazetidin-2-yl)-1H-1,2,4-triazole-1-
carbonyl}benzoic acid
3-{5-[(5-chlorothiophen-2-yl)methoxy]-1-(1,3-thiazole-4-carbonyl)-1H-1,2,4-triazol-3-
yl}-1-methanesulfonyl-4-methylpyrrolidin-2-one
3-{5-[(5-chlorothiophen-2-yl)methoxy]-1-(1,3-thiazole-4-carbonyl)-1H-1,2,4-triazol-3-
yl}-4-methyl-1-(pyrrolidine-1-sulfonyl)piperidin-2-one
3-{5-[(5-chlorothiophen-2-yl)methoxy]-1-(2,2-dimethylpropanoyl)-1H-1,2,4-triazol-3-yl}-
1-(2,2-dimethylpropanoyl)-4-(trifluoromethyl)piperidin-2-one
3-{5-[(5-chlorothiophen-2-yl)methoxy]-1-(2,2-dimethylpropanoyl)-1H-1,2,4-triazol-3-yl}-
1-[2-(morpholin-4-yl)acetyl]-4-(trifluoromethyl)pyrrolidine-2-carboxylic acid
3-{5-[(5-chlorothiophen-2-yl)methoxy]-1-(2,2-dimethylpropanoyl)-1H-1,2,4-triazol-3-yl}-
4-(trifluoromethyl)azetidin-2-one
3-{5-[(5-chlorothiophen-2-yl)methoxy]-1-(2-fluorobenzoyl)-1H-1,2,4-triazol-3-yl}-1-
(pyrrolidine-1-sulfonyl)-4-(trifluoromethyl)azetidin-2-one
3-{5-[(5-chlorothiophen-2-yl)methoxy]-1-(2-methoxybenzoyl)-1H-1,2,4-triazol-3-yl}-1-
(3-hydroxypyrrolidine-1-carbonyl)-4-(trifluoromethyl)azetidin-2-one
3-{5-[(5-chlorothiophen-2-yl)methoxy]-1-(2-methoxybenzoyl)-1H-1,2,4-triazol-3-yl}-
N,N-dimethyl-2-(trifluoromethyl)piperidine-1-carboxamide
3-{5-[(5-chlorothiophen-2-yl)methoxy]-1-(3-hydroxy-2,2-dimethylpropanoyl)-1H-1,2,4-
triazol-3-yl}-N,N,4-trimethyl-2-oxopyrrolidine-1-carboxamide
3-{5-[(5-chlorothiophen-2-yl)methoxy]-1-(thiophene-2-carbonyl)-1H-1,2,4-triazol-3-yl}-
1-[2-(morpholin-4-yl)acetyl]piperidin-2-one
3-{5-[(5-chlorothiophen-2-yl)methoxy]-1-(thiophene-2-carbonyl)-1H-1,2,4-triazol-3-yl}-
N,N-dimethyl-2-oxopyrrolidine-1-sulfonamide
3-{5-[(5-chlorothiophen-2-yl)methoxy]-3-[1-(dimethylsulfamoyl)-2-oxopiperidin-4-yl]-
1H-1,2,4-triazole-1-carbonyl}benzoic acid
3-{5-[(5-chlorothiophen-2-yl)methoxy]-3-[1-(morpholine-4-carbonyl)-6-oxopiperidin-3-
yl]-1H-1,2,4-triazole-1-carbonyl}benzoic acid
3-{5-[(5-chlorothiophen-2-yl)methoxy]-3-{1-[(3-hydroxypyrrolidin-1-
yl)sulfonyl]azetidin-2-yl}-1H-1,2,4-triazole-1-carbonyl}benzoic acid
4-(1-benzoyl-5-{[(5-chlorothiophen-2-yl)methyl](methyl)amino}-1H-1,2,4-triazol-3-yl)-1-
(pyrrolidine-1-carbonyl)pyrrolidin-2-ol
4-(1-benzoyl-5-{[(5-chlorothiophen-2-yl)methyl](methyl)amino}-1H-1,2,4-triazol-3-yl)-
N,N-dimethyl-3-oxopiperidine-1-carboxamide
4-(5-{[(4-fluorophenyl)methyl](methyl)amino}-1-(4-methylfuran-3-carbonyl)-1H-1,2,4-
triazol-3-yl)-3-methyl-1-(pyrrolidine-1-carbonyl)piperidin-2-one
4-(5-{[(4-fluorophenyl)methyl](methyl)amino}-1-(furan-2-carbonyl)-1H-1,2,4-triazol-3-
yl)-1-(pyrrolidine-1-sulfonyl)-3-(trifluoromethyl)pyrrolidin-2-one
4-(5-{[(4-fluorophenyl)methyl](methyl)amino}-1-(furan-2-carbonyl)-1H-1,2,4-triazol-3-
yl)-1-methanesulfonyl-5-(trifluoromethyl)piperidin-3-one
4-(5-{[(4-fluorophenyl)methyl]amino}-1-(2-methylfuran-3-carbonyl)-1H-1,2,4-triazol-3-
yl)-1-methanesulfonylpyrrolidin-2-ol
4-(5-{[(4-fluorophenyl)methyl]amino}-1-(2-methylfuran-3-carbonyl)-1H-1,2,4-triazol-3-
yl)-N,N-dimethyl-2-oxopiperidine-1-carboxamide
4-(5-{[(4-fluorophenyl)methyl]amino}-1-(3-methoxy-2,2-dimethylpropanoyl)-1H-1,2,4-
triazol-3-yl)-N,N,3-trimethyl-5-oxopiperidine-1-sulfonamide
4-(5-{[(4-fluorophenyl)methyl]amino}-1-(4-methylfuran-3-carbonyl)-1H-1,2,4-triazol-3-
yl)-N,N,3-trimethyl-2-oxopyrrolidine-1-carboxamide
4-(5-{[(4-fluorophenyl)methyl]amino}-1-(5-methylfuran-3-carbonyl)-1H-1,2,4-triazol-3-
yl)-1-[(3-hydroxypyrrolidin-1-yl)sulfonyl]-3-(trifluoromethyl)piperidin-2-one
4-(5-{[(4-fluorophenyl)methyl]amino}-1-(furan-2-carbonyl)-1H-1,2,4-triazol-3-yl)-1-[2-
(morpholin-4-yl)acetyl]-5-(trifluoromethyl)piperidin-3-one
4-(5-{[(4-fluorophenyl)methyl]sulfanyl}-1-(1,3-thiazole-4-carbonyl)-1H-1,2,4-triazol-3-
yl)-N,N,3-trimethyl-2-oxopyrrolidine-1-carboxamide
4-(5-{[(4-fluorophenyl)methyl]sulfanyl}-1-(2-methoxybenzoyl)-1H-1,2,4-triazol-3-yl)-1-
[(3-hydroxypyrrolidin-1-yl)sulfonyl]-3-(trifluoromethyl)piperidin-2-one
4-(5-{[(4-fluorophenyl)methyl]sulfanyl}-1-(3-hydroxy-2,2-dimethylpropanoyl)-1H-1,2,4-
triazol-3-yl)-3-methyl-1-(morpholine-4-carbonyl)pyrrolidin-2-one
4-(5-{[(4-fluorophenyl)methyl]sulfanyl}-1-(3-hydroxy-2,2-dimethylpropanoyl)-1H-1,2,4-
triazol-3-yl)-N,N,3-trimethyl-5-oxopiperidine-1-sulfonamide
4-(5-{[(4-fluorophenyl)methyl]sulfanyl}-1H-1,2,4-triazol-3-yl)-1-(3-hydroxypyrrolidine-
1-carbonyl)-5-methylpiperidin-3-one
4-(5-{[(4-fluorophenyl)methyl]sulfanyl}-1H-1,2,4-triazol-3-yl)-1-(3-hydroxypyrrolidine-
1-carbonyl)-5-methylpyrrolidin-3-one
4-(5-{[(5-chlorothiophen-2-yl)methyl](methyl)amino}-1-(2-fluorobenzoyl)-1H-1,2,4-
triazol-3-yl)-1-methanesulfonyl-5-(trifluoromethyl)piperidin-3-one
4-(5-{[(5-chlorothiophen-2-yl)methyl](methyl)amino}-1-(3-hydroxy-2,2-
dimethylpropanoyl)-1H-1,2,4-triazol-3-yl)-1-(2,2-dimethylpropanoyl)-3-methylpyrrolidin-
2-one
4-(5-{[(5-chlorothiophen-2-yl)methyl](methyl)amino}-1-(thiophene-2-carbonyl)-1H-1,2,4-
triazol-3-yl)-1-[(3-hydroxypyrrolidin-1-yl)sulfonyl]azetidin-2-one
4-(5-{[(5-chlorothiophen-2-yl)methyl]amino}-1-(2-methylfuran-3-carbonyl)-1H-1,2,4-
triazol-3-yl)-1-(2,2-dimethylpropanoyl)azetidin-2-one
4-(5-{[(5-chlorothiophen-2-yl)methyl]amino}-1-(5-methylfuran-3-carbonyl)-1H-1,2,4-
triazol-3-yl)-5-(trifluoromethyl)piperidin-3-one
4-(5-{[(5-chlorothiophen-2-yl)methyl]amino}-1-(furan-2-carbonyl)-1H-1,2,4-triazol-3-yl)-
1-[(3-hydroxypyrrolidin-1-yl)sulfonyl]-3-(trifluoromethyl)pyrrolidin-2-one
4-(5-{[(5-chlorothiophen-2-yl)methyl]amino}-1-(furan-2-carbonyl)-1H-1,2,4-triazol-3-yl)-
1-[2-(morpholin-4-yl)-2-oxoethyl]-3-(trifluoromethyl)azetidin-2-one
4-(5-{[(5-chlorothiophen-2-yl)methyl]amino}-1-(furan-3-carbonyl)-1H-1,2,4-triazol-3-yl)-
1-(morpholine-4-carbonyl)-5-(trifluoromethyl)pyrrolidin-2-ol
4-(5-{[(5-chlorothiophen-2-yl)methyl]amino}-1-(thiophene-3-carbonyl)-1H-1,2,4-triazol-
3-yl)-1-(3-hydroxypyrrolidine-1-carbonyl)-5-methylpiperidin-3-one
4-(5-{[(5-chlorothiophen-2-yl)methyl]amino}-1-(thiophene-3-carbonyl)-1H-1,2,4-triazol-
3-yl)-1-(3-hydroxypyrrolidine-1-carbonyl)-5-methylpyrrolidin-3-one
4-(5-{[(5-chlorothiophen-2-yl)methyl]amino}-1-(thiophene-3-carbonyl)-1H-1,2,4-triazol-
3-yl)-5-methylpyrrolidin-3-one
4-(5-{[(5-chlorothiophen-2-yl)methyl]sulfanyl}-1-(2-methylfuran-3-carbonyl)-1H-1,2,4-
triazol-3-yl)-1-[2-(morpholin-4-yl)acetyl]pyrrolidin-2-one
4-(5-{[(5-chlorothiophen-2-yl)methyl]sulfanyl}-1-(furan-2-carbonyl)-1H-1,2,4-triazol-3-
yl)-1-(pyrrolidine-1-sulfonyl)-3-(trifluoromethyl)azetidin-2-one
4-(5-{[(5-chlorothiophen-2-yl)methyl]sulfanyl}-1-(furan-2-carbonyl)-1H-1,2,4-triazol-3-
yl)-1-methanesulfonyl-3-(trifluoromethyl)azetidin-2-one
4-(5-{[(5-chlorothiophen-2-yl)methyl]sulfanyl}-1-(furan-3-carbonyl)-1H-1,2,4-triazol-3-
yl)-1-methanesulfonyl-3-(trifluoromethyl)piperidin-2-one
4-(5-{[(5-chlorothiophen-2-yl)methyl]sulfanyl}-1-(thiophene-3-carbonyl)-1H-1,2,4-
triazol-3-yl)-3-methyl-1-(morpholine-4-carbonyl)piperidin-2-one
4-(5-{[4-(aminomethyl)phenyl]methoxy}-1-(2-chlorobenzoyl)-1H-1,2,4-triazol-3-
yl)pyrrolidin-2-one
4-(5-{[4-(aminomethyl)phenyl]methoxy}-1-(2-methylfuran-3-carbonyl)-1H-1,2,4-triazol-
3-yl)-1-(morpholine-4-carbonyl)piperidin-3-one
4-(5-{[4-(aminomethyl)phenyl]methoxy}-1-(2-methylfuran-3-carbonyl)-1H-1,2,4-triazol-
3-yl)-1-[2-(morpholin-4-yl)-2-oxoethyl]piperidin-2-one
4-(5-{[4-(aminomethyl)phenyl]methoxy}-1-(3-methoxy-2,2-dimethylpropanoyl)-1H-
1,2,4-triazol-3-yl)-N,N,3-trimethyl-2-oxopyrrolidine-1-sulfonamide
4-(5-{[4-(aminomethyl)phenyl]methoxy}-1-(5-methylfuran-3-carbonyl)-1H-1,2,4-triazol-
3-yl)-1-(pyrrolidine-1-carbonyl)-5-(trifluoromethyl)pyrrolidin-3-one
4-(5-{[4-(aminomethyl)phenyl]methoxy}-1-(furan-2-carbonyl)-1H-1,2,4-triazol-3-yl)-1-
(pyrrolidine-1-carbonyl)-3-(trifluoromethyl)azetidin-2-one
4-(5-{[4-(aminomethyl)phenyl]methoxy}-1-(furan-3-carbonyl)-1H-1,2,4-triazol-3-yl)-1-
[2-(morpholin-4-yl)acetyl]-3-(trifluoromethyl)azetidin-2-one
4-(5-{[4-(aminomethyl)phenyl]methoxy}-1-(thiophene-3-carbonyl)-1H-1,2,4-triazol-3-yl)-
N,N,3-trimethylpiperidine-1-carboxamide
4-({[1-(2,2-dimethylpropanoyl)-3-[1-(2,2-dimethylpropanoyl)-2-
(trifluoromethyl)piperidin-3-yl]-1H-1,2,4-triazol-5-yl]oxy}methyl)benzene-1-
carboximidamide
4-({[1-(2,2-dimethylpropanoyl)-3-[1-(2,2-dimethylpropanoyl)-3-oxo-5-
(trifluoromethyl)piperidin-4-yl]-1H-1,2,4-triazol-5-yl]oxy}methyl)benzene-1-
carboximidamide
4-({[1-(2,2-dimethylpropanoyl)-3-[1-methanesulfonyl-2-oxo-3-(trifluoromethyl)piperidin-
4-yl]-1H-1,2,4-triazol-5-yl]amino}methyl)benzene-1-carboximidamide
4-({[1-(2,2-dimethylpropanoyl)-3-[4-(dimethylsulfamoyl)-3-(trifluoromethyl)piperazin-2-
yl]-1H-1,2,4-triazol-5-yl]amino}methyl)benzene-1-carboximidamide
4-({[1-(2,2-dimethylpropanoyl)-3-[4-methanesulfonyl-3-(trifluoromethyl)piperazin-2-yl]-
1H-1,2,4-triazol-5-yl]amino}methyl)benzene-1-carboximidamide
4-({[1-(2,2-dimethylpropanoyl)-3-[5-hydroxy-1-(morpholine-4-carbonyl)-2-
(trifluoromethyl)pyrrolidin-3-yl]-1H-1,2,4-triazol-5-yl]amino}methyl)benzene-1-
carboximidamide
4-({[1-(2,2-dimethylpropanoyl)-3-{1-[2-(morpholin-4-yl)acetyl]-4-oxo-3-
(trifluoromethyl)azetidin-2-yl}-1H-1,2,4-triazol-5-yl]oxy}methyl)benzene-1-
carboximidamide
4-({[1-(2-chlorobenzoyl)-3-(morpholin-3-yl)-1H-1,2,4-triazol-5-
yl]sulfanyl}methyl)benzene-1-carboximidamide
4-({[1-(2-chlorobenzoyl)-3-[1-(dimethylsulfamoyl)pyrrolidin-3-yl]-1H-1,2,4-triazol-5-
yl](methyl)amino}methyl)benzene-1-carboximidamide
4-({[1-(2-chlorobenzoyl)-3-[2-oxo-1-(pyrrolidine-1-sulfonyl)piperidin-4-yl]-1H-1,2,4-
triazol-5-yl](methyl)amino}methyl)benzene-1-carboximidamide
4-({[1-(2-chlorobenzoyl)-3-{1-[2-(morpholin-4-yl)-2-oxoethyl]-5-oxopyrrolidin-3-yl}-1H-
1,2,4-triazol-5-yl](methyl)amino}methyl)benzene-1-carboximidamide
4-({[1-(2-chlorobenzoyl)-3-{1-[2-(morpholin-4-yl)acetyl]-2-oxopiperidin-4-yl}-1H-1,2,4-
triazol-5-yl]sulfanyl}methyl)benzene-1-carboximidamide
4-({[1-(2-fluorobenzoyl)-3-[1-(morpholine-4-carbonyl)-2-(trifluoromethyl)azetidin-3-yl]-
1H-1,2,4-triazol-5-yl]amino}methyl)benzene-1-carboximidamide
4-({[1-(2-fluorobenzoyl)-3-[4-(morpholine-4-carbonyl)-3-(trifluoromethyl)piperazin-2-yl]-
1H-1,2,4-triazol-5-yl]oxy}methyl)benzene-1-carboximidamide
4-({[1-(2-fluorobenzoyl)-3-[4-oxo-1-(pyrrolidine-1-carbonyl)-3-(trifluoromethyl)azetidin-
2-yl]-1H-1,2,4-triazol-5-yl]oxy}methyl)benzene-1-carboximidamide
4-({[1-(2-fluorobenzoyl)-3-{1-[(3-hydroxypyrrolidin-1-yl)sulfonyl]-5-oxo-4-
(trifluoromethyl)pyrrolidin-3-yl}-1H-1,2,4-triazol-5-yl]amino}methyl)benzene-1-
carboximidamide
4-({[1-(2-fluorobenzoyl)-3-{1-[2-(morpholin-4-yl)-2-oxoethyl]-2-
(trifluoromethyl)piperidin-3-yl}-1H-1,2,4-triazol-5-yl]amino}methyl)benzene-1-
carboximidamide
4-({[1-(2-methoxybenzoyl)-3-[1-(pyrrolidine-1-carbonyl)-4-(trifluoromethyl)piperidin-3-
yl]-1H-1,2,4-triazol-5-yl]amino}methyl)benzene-1-carboximidamide
4-({[1-(2-methoxybenzoyl)-3-[1-(pyrrolidine-1-sulfonyl)-3-(trifluoromethyl)azetidin-2-
yl]-1H-1,2,4-triazol-5-yl]oxy}methyl)benzene-1-carboximidamide
4-({[1-(2-methoxybenzoyl)-3-[3-oxo-5-(trifluoromethyl)piperidin-4-yl]-1H-1,2,4-triazol-
5-yl]amino}methyl)benzene-1-carboximidamide
4-({[1-(2-methoxybenzoyl)-3-[4-(pyrrolidine-1-carbonyl)-3-(trifluoromethyl)piperazin-2-
yl]-1H-1,2,4-triazol-5-yl]amino}methyl)benzene-1-carboximidamide
4-({[1-(2-methoxybenzoyl)-3-[4-oxo-1-(pyrrolidine-1-carbonyl)-2-
(trifluoromethyl)pyrrolidin-3-yl]-1H-1,2,4-triazol-5-yl]oxy}methyl)benzene-1-
carboximidamide
4-({[1-(2-methylfuran-3-carbonyl)-3-[1-(morpholine-4-carbonyl)azetidin-2-yl]-1H-1,2,4-
triazol-5-yl]sulfanyl}methyl)benzene-1-carboximidamide
4-({[1-(3-hydroxy-2,2-dimethylpropanoyl)-3-(2-methylpyrrolidin-3-yl)-1H-1,2,4-triazol-5-
yl]oxy}methyl)benzene-1-carboximidamide
4-({[1-(3-hydroxy-2,2-dimethylpropanoyl)-3-[1-(3-hydroxypyrrolidine-1-carbonyl)-4-
methyl-2-oxopyrrolidin-3-yl]-1H-1,2,4-triazol-5-yl]amino}methyl)benzene-1-
carboximidamide
4-({[1-(3-hydroxy-2,2-dimethylpropanoyl)-3-[4-methyl-1-(pyrrolidine-1-
sulfonyl)pyrrolidin-3-yl]-1H-1,2,4-triazol-5-yl]amino}methyl)benzene-1-carboximidamide
4-({[1-(3-hydroxy-2,2-dimethylpropanoyl)-3-{1-[(3-hydroxypyrrolidin-1-yl)sulfonyl]-2-
methylpiperidin-3-yl}-1H-1,2,4-triazol-5-yl]amino}methyl)benzene-1-carboximidamide
4-({[1-(3-hydroxy-2,2-dimethylpropanoyl)-3-{1-[(3-hydroxypyrrolidin-1-yl)sulfonyl]-4-
methylpyrrolidin-3-yl}-1H-1,2,4-triazol-5-yl]oxy}methyl)benzene-1-carboximidamide
4-({[1-(3-methoxy-2,2-dimethylpropanoyl)-3-(4-methyl-2-oxopyrrolidin-3-yl)-1H-1,2,4-
triazol-5-yl]sulfanyl}methyl)benzene-1-carboximidamide
4-({[1-(3-methoxy-2,2-dimethylpropanoyl)-3-[2-methyl-1-(pyrrolidine-1-
sulfonyl)pyrrolidin-3-yl]-1H-1,2,4-triazol-5-yl](methyl)amino}methyl)benzene-1-
carboximidamide
4-({[1-(3-methoxy-2,2-dimethylpropanoyl)-3-{2-methyl-1-[2-(morpholin-4-yl)acetyl]-4-
oxopyrrolidin-3-yl}-1H-1,2,4-triazol-5-yl](methyl)amino}methyl)benzene-1-
carboximidamide
4-({[1-(3-methoxy-2,2-dimethylpropanoyl)-3-{2-methyl-1-[2-(morpholin-4-
yl)acetyl]azetidin-3-yl}-1H-1,2,4-triazol-5-yl]sulfanyl}methyl)benzene-1-
carboximidamide
4-({[1-(5-methylfuran-3-carbonyl)-3-[2-(trifluoromethyl)oxetan-3-yl]-1H-1,2,4-triazol-5-
yl]sulfanyl}methyl)benzene-1-carboximidamide
4-({[1-(5-methylfuran-3-carbonyl)-3-{1-[2-(morpholin-4-yl)-2-oxoethyl]-4-oxo-2-
(trifluoromethyl)pyrrolidin-3-yl}-1H-1,2,4-triazol-5-yl]sulfanyl}methyl)benzene-1-
carboximidamide
4-({[1-(furan-2-carbonyl)-3-[1-(3-hydroxypyrrolidine-1-carbonyl)-3-
(trifluoromethyl)piperidin-4-yl]-1H-1,2,4-triazol-5-yl]sulfanyl}methyl)benzene-1-
carboximidamide
4-({[1-(furan-2-carbonyl)-3-[3-(trifluoromethyl)azetidin-2-yl]-1H-1,2,4-triazol-5-
yl](methyl)amino}methyl)benzene-1-carboximidamide
4-({[1-(furan-2-carbonyl)-3-[4-oxo-3-(trifluoromethyl)azetidin-2-yl]-1H-1,2,4-triazol-5-
yl]sulfanyl}methyl)benzene-1-carboximidamide
4-({[1-(furan-2-carbonyl)-3-{1-[(3-hydroxypyrrolidin-1-yl)sulfonyl]-3-oxo-5-
(trifluoromethyl)piperidin-4-yl}-1H-1,2,4-triazol-5-yl](methyl)amino}methyl)benzene-1-
carboximidamide
4-({[1-(furan-2-carbonyl)-3-{1-[2-(morpholin-4-yl)-2-oxoethyl]-3-oxo-5-
(trifluoromethyl)piperidin-4-yl}-1H-1,2,4-triazol-5-yl]sulfanyl}methyl)benzene-1-
carboximidamide
4-({[1-(furan-3-carbonyl)-3-[1-(3-hydroxypyrrolidine-1-carbonyl)-4-
(trifluoromethyl)piperidin-3-yl]-1H-1,2,4-triazol-5-yl]sulfanyl}methyl)benzene-1-
carboximidamide
4-({[1-(furan-3-carbonyl)-3-[1-(morpholine-4-carbonyl)-2-(trifluoromethyl)pyrrolidin-3-
yl]-1H-1,2,4-triazol-5-yl](methyl)amino}methyl)benzene-1-carboximidamide
4-({[1-(furan-3-carbonyl)-3-[1-(pyrrolidine-1-carbonyl)-2-(trifluoromethyl)pyrrolidin-3-
yl]-1H-1,2,4-triazol-5-yl](methyl)amino}methyl)benzene-1-carboximidamide
4-({[1-(furan-3-carbonyl)-3-[1-methanesulfonyl-2-(trifluoromethyl)pyrrolidin-3-yl]-1H-
1,2,4-triazol-5-yl]sulfanyl}methyl)benzene-1-carboximidamide
4-({[1-(furan-3-carbonyl)-3-[1-methanesulfonyl-4-oxo-2-(trifluoromethyl)pyrrolidin-3-yl]-
1H-1,2,4-triazol-5-yl](methyl)amino}methyl)benzene-1-carboximidamide
4-({[1-(furan-3-carbonyl)-3-{1-[(3-hydroxypyrrolidin-1-yl)sulfonyl]-4-
(trifluoromethyl)piperidin-3-yl}-1H-1,2,4-triazol-5-yl]sulfanyl}methyl)benzene-1-
carboximidamide
4-({[1-benzoyl-3-(5-oxopyrrolidin-3-yl)-1H-1,2,4-triazol-5-yl]oxy}methyl)benzene-1-
carboximidamide
4-({[3-(1-methanesulfonyl-2-methylpiperidin-3-yl)-1-(4-methylfuran-3-carbonyl)-1H-
1,2,4-triazol-5-yl]sulfanyl}methyl)benzene-1-carboximidamide
4-({[3-(1-methanesulfonyl-4-methyl-2-oxopyrrolidin-3-yl)-1-(4-methylfuran-3-carbonyl)-
1H-1,2,4-triazol-5-yl](methyl)amino}methyl)benzene-1-carboximidamide
4-({[3-(1-methanesulfonylazetidin-2-yl)-1-(2-methylfuran-3-carbonyl)-1H-1,2,4-triazol-5-
yl]sulfanyl}methyl)benzene-1-carboximidamide
4-({[3-(2-methyl-4-oxopyrrolidin-3-yl)-1H-1,2,4-triazol-5-yl]amino}methyl)benzene-1-
carboximidamide
4-({methyl[1-(2-methylfuran-3-carbonyl)-3-{1-[2-(morpholin-4-yl)-2-oxoethyl]pyrrolidin-
3-yl}-1H-1,2,4-triazol-5-yl]amino}methyl)benzene-1-carboximidamide
4-({methyl[1-(5-methylfuran-3-carbonyl)-3-[2-oxo-1-(pyrrolidine-1-carbonyl)-4-
(trifluoromethyl)pyrrolidin-3-yl]-1H-1,2,4-triazol-5-yl]amino}methyl)benzene-1-
carboximidamide
4-({methyl[1-(5-methylfuran-3-carbonyl)-3-{1-[2-(morpholin-4-yl)-2-oxoethyl]-2-
(trifluoromethyl)pyrrolidin-3-yl}-1H-1,2,4-triazol-5-yl]amino}methyl)benzene-1-
carboximidamide
4-({methyl[1-(5-methylfuran-3-carbonyl)-3-{1-[2-(morpholin-4-yl)acetyl]-2-oxo-4-
(trifluoromethyl)azetidin-3-yl}-1H-1,2,4-triazol-5-yl]amino}methyl)benzene-1-
carboximidamide
4-({methyl[3-(2-methyloxolan-3-yl)-1-(thiophene-3-carbonyl)-1H-1,2,4-triazol-5-
yl]amino}methyl)benzene-1-carboximidamide
4-({methyl[3-(3-methyl-2-oxopiperidin-4-yl)-1-(4-methylfuran-3-carbonyl)-1H-1,2,4-
triazol-5-yl]amino}methyl)benzene-1-carboximidamide
4-({methyl[3-(3-methyloxetan-2-yl)-1-(thiophene-3-carbonyl)-1H-1,2,4-triazol-5-
yl]amino}methyl)benzene-1-carboximidamide
4-[({1-benzoyl-3-[1-(2,2-dimethylpropanoyl)-2-oxoazetidin-3-yl]-1H-1,2,4-triazol-5-
yl}oxy)methyl]benzene-1-carboximidamide
4-[({1-benzoyl-3-[1-(dimethylsulfamoyl)azetidin-3-yl]-1H-1,2,4-triazol-5-
yl}oxy)methyl]benzene-1-carboximidamide
4-[({1-benzoyl-3-[1-(pyrrolidine-1-sulfonyl)piperidin-3-yl]-1H-1,2,4-triazol-5-
yl}amino)methyl]benzene-1-carboximidamide
4-[({1-benzoyl-3-[2-oxo-1-(pyrrolidine-1-carbonyl)azetidin-3-yl]-1H-1,2,4-triazol-5-
yl}amino)methyl]benzene-1-carboximidamide
4-[({3-[1-(2,2-dimethylpropanoyl)-2-methylazetidin-3-yl]-1H-1,2,4-triazol-5-
yl}oxy)methyl]benzene-1-carboximidamide
4-[({3-[1-(2,2-dimethylpropanoyl)-2-methylpyrrolidin-3-yl]-1-(thiophene-3-carbonyl)-1H-
1,2,4-triazol-5-yl}sulfanyl)methyl]benzene-1-carboximidamide
4-[({3-[1-(2,2-dimethylpropanoyl)-2-oxo-3-(trifluoromethyl)piperidin-4-yl]-1-(2-
fluorobenzoyl)-1H-1,2,4-triazol-5-yl}oxy)methyl]benzene-1-carboximidamide
4-[({3-[1-(2,2-dimethylpropanoyl)-2-oxo-4-(trifluoromethyl)piperidin-3-yl]-1-(furan-3-
carbonyl)-1H-1,2,4-triazol-5-yl}(methyl)amino)methyl]benzene-1-carboximidamide
4-[({3-[1-(2,2-dimethylpropanoyl)-3-(trifluoromethyl)piperazin-2-yl]-1-(5-methylfuran-3-
carbonyl)-1H-1,2,4-triazol-5-yl}sulfanyl)methyl]benzene-1-carboximidamide
4-[({3-[1-(2,2-dimethylpropanoyl)-4-oxopyrrolidin-3-yl]-1-(2-methylfuran-3-carbonyl)-
1H-1,2,4-triazol-5-yl}sulfanyl)methyl]benzene-1-carboximidamide
4-[({3-[1-(2,2-dimethylpropanoyl)-6-oxopiperidin-3-yl]-1-(thiophene-2-carbonyl)-1H-
1,2,4-triazol-5-yl}oxy)methyl]benzene-1-carboximidamide
4-[({3-[1-(3-hydroxypyrrolidine-1-carbonyl)-2-methylpiperidin-3-yl]-1-(4-methylfuran-3-
carbonyl)-1H-1,2,4-triazol-5-yl}sulfanyl)methyl]benzene-1-carboximidamide
4-[({3-[1-(3-hydroxypyrrolidine-1-carbonyl)-2-oxo-3-(trifluoromethyl)piperidin-4-yl]-1-
(5-methylfuran-3-carbonyl)-1H-1,2,4-triazol-5-yl}sulfanyl)methyl]benzene-1-
carboximidamide
4-[({3-[1-(3-hydroxypyrrolidine-1-carbonyl)-2-oxo-4-(trifluoromethyl)azetidin-3-yl]-1-(5-
methylfuran-3-carbonyl)-1H-1,2,4-triazol-5-yl}(methyl)amino)methyl]benzene-1-
carboximidamide
4-[({3-[1-(3-hydroxypyrrolidine-1-carbonyl)-4-methyl-2-oxopiperidin-3-yl]-1-(thiophene-
3-carbonyl)-1H-1,2,4-triazol-5-yl}sulfanyl)methyl]benzene-1-carboximidamide
4-[({3-[1-(dimethylsulfamoyl)-2-oxoazetidin-3-yl]-1-(2-methylfuran-3-carbonyl)-1H-
1,2,4-triazol-5-yl}(methyl)amino)methyl]benzene-1-carboximidamide
4-[({3-[1-(dimethylsulfamoyl)-2-oxopiperidin-4-yl]-1-(2-methylfuran-3-carbonyl)-1H-
1,2,4-triazol-5-yl}(methyl)amino)methyl]benzene-1-carboximidamide
4-[({3-[1-(dimethylsulfamoyl)-3-(trifluoromethyl)piperidin-4-yl]-1-(furan-2-carbonyl)-
1H-1,2,4-triazol-5-yl}(methyl)amino)methyl]benzene-1-carboximidamide
4-[({3-[1-(dimethylsulfamoyl)-3-methylazetidin-2-yl]-1-(3-hydroxy-2,2-
dimethylpropanoyl)-1H-1,2,4-triazol-5-yl}oxy)methyl]benzene-1-carboximidamide
4-[({3-[1-(dimethylsulfamoyl)-3-methylpiperazin-2-yl]-1-(1,3-thiazole-4-carbonyl)-1H-
1,2,4-triazol-5-yl}amino)methyl]benzene-1-carboximidamide
4-[({3-[1-(dimethylsulfamoyl)-4-methyl-2-oxopiperidin-3-yl]-1-(1,3-thiazole-4-carbonyl)-
1H-1,2,4-triazol-5-yl}oxy)methyl]benzene-1-carboximidamide
4-[({3-[1-(dimethylsulfamoyl)-4-methyl-6-oxopiperidin-3-yl]-1-(1,3-thiazole-4-carbonyl)-
1H-1,2,4-triazol-5-yl}amino)methyl]benzene-1-carboximidamide
4-[({3-[1-(dimethylsulfamoyl)-4-methylpiperidin-3-yl]-1-(3-hydroxy-2,2-
dimethylpropanoyl)-1H-1,2,4-triazol-5-yl}amino)methyl]benzene-1-carboximidamide
4-[({3-[1-(dimethylsulfamoyl)-5-hydroxy-2-(trifluoromethyl)pyrrolidin-3-yl]-1-(2-
fluorobenzoyl)-1H-1,2,4-triazol-5-yl}oxy)methyl]benzene-1-carboximidamide
4-[({3-[1-(morpholine-4-carbonyl)-4-oxoazetidin-2-yl]-1-(thiophene-2-carbonyl)-1H-
1,2,4-triazol-5-yl}oxy)methyl]benzene-1-carboximidamide
4-[({3-[1-(morpholine-4-carbonyl)-4-oxopyrrolidin-3-yl]-1-(thiophene-2-carbonyl)-1H-
1,2,4-triazol-5-yl}amino)methyl]benzene-1-carboximidamide
4-[({3-[1-(pyrrolidine-1-sulfonyl)piperidin-3-yl]-1-(thiophene-2-carbonyl)-1H-1,2,4-
triazol-5-yl}amino)methyl]benzene-1-carboximidamide
4-[({3-[2-methyl-1-(morpholine-4-carbonyl)piperidin-3-yl]-1-(1,3-thiazole-4-carbonyl)-
1H-1,2,4-triazol-5-yl}amino)methyl]benzene-1-carboximidamide
4-[({3-[5-hydroxy-2-methyl-1-(pyrrolidine-1-sulfonyl)pyrrolidin-3-yl]-1-(4-methylfuran-
3-carbonyl)-1H-1,2,4-triazol-5-yl}(methyl)amino)methyl]benzene-1-carboximidamide
4-[({3-[5-oxo-1-(pyrrolidine-1-carbonyl)pyrrolidin-3-yl]-1-(thiophene-2-carbonyl)-1H-
1,2,4-triazol-5-yl}oxy)methyl]benzene-1-carboximidamide
4-[1-(2-chlorobenzoyl)-5-{[(4-fluorophenyl)methyl]amino}-1H-1,2,4-triazol-3-yl]-N,N-
dimethyl-3-oxopiperidine-1-carboxamide
4-[1-(2-chlorobenzoyl)-5-{[(5-chlorothiophen-2-yl)methyl]sulfanyl}-1H-1,2,4-triazol-3-
yl]-1-(pyrrolidine-1-sulfonyl)pyrrolidin-3-one
4-[1-(2-fluorobenzoyl)-5-[(4-fluorophenyl)methoxy]-1H-1,2,4-triazol-3-yl]-3-
(trifluoromethyl)azetidin-2-one
4-[1-(2-fluorobenzoyl)-5-{[(4-fluorophenyl)methyl]sulfanyl}-1H-1,2,4-triazol-3-yl]-1-[2-
(morpholin-4-yl)-2-oxoethyl]-3-(trifluoromethyl)azetidin-2-one
4-[1-(2-fluorobenzoyl)-5-{[(4-fluorophenyl)methyl]sulfanyl}-1H-1,2,4-triazol-3-yl]-1-[2-
(morpholin-4-yl)acetyl]-5-(trifluoromethyl)piperidin-3-one
4-[3-(5-{[(5-chlorothiophen-2-yl)methyl]sulfanyl}-1-(furan-2-carbonyl)-1H-1,2,4-triazol-
3-yl)-2-(trifluoromethyl)azetidine-1-carbonyl]morpholine
4-[5-({[4-(aminomethyl)phenyl]methyl}(methyl)amino)-1-(1,3-thiazole-4-carbonyl)-1H-
1,2,4-triazol-3-yl]-5-methyl-1-(pyrrolidine-1-carbonyl)piperidin-3-one
4-[5-({[4-(aminomethyl)phenyl]methyl}(methyl)amino)-1-(1,3-thiazole-4-carbonyl)-1H-
1,2,4-triazol-3-yl]-5-methyl-1-(pyrrolidine-1-sulfonyl)pyrrolidin-2-ol
4-[5-({[4-(aminomethyl)phenyl]methyl}(methyl)amino)-1-(2,2-dimethylpropanoyl)-1H-
1,2,4-triazol-3-yl]-1-methanesulfonyl-5-(trifluoromethyl)pyrrolidin-3-one
4-[5-({[4-(aminomethyl)phenyl]methyl}(methyl)amino)-1-(2-fluorobenzoyl)-1H-1,2,4-
triazol-3-yl]-N,N-dimethyl-3-(trifluoromethyl)piperidine-1-sulfonamide
4-[5-({[4-(aminomethyl)phenyl]methyl}(methyl)amino)-1-(3-hydroxy-2,2-
dimethylpropanoyl)-1H-1,2,4-triazol-3-yl]-N,N,3-trimethyl-2-oxopyrrolidine-1-
sulfonamide
4-[5-({[4-(aminomethyl)phenyl]methyl}(methyl)amino)-1-(thiophene-2-carbonyl)-1H-
1,2,4-triazol-3-yl]-1-methanesulfonylpyrrolidin-2-one
4-[5-({[4-(aminomethyl)phenyl]methyl}(methyl)amino)-1-benzoyl-1H-1,2,4-triazol-3-yl]-
1-[2-(morpholin-4-yl)-2-oxoethyl]pyrrolidin-2-one
4-[5-({[4-(aminomethyl)phenyl]methyl}(methyl)amino)-1H-1,2,4-triazol-3-yl]-N,N,3-
trimethylpiperidine-1-carboxamide
4-[5-({[4-(aminomethyl)phenyl]methyl}amino)-1-(2-methylfuran-3-carbonyl)-1H-1,2,4-
triazol-3-yl]-1-(3-hydroxypyrrolidine-1-carbonyl)pyrrolidin-2-one
4-[5-({[4-(aminomethyl)phenyl]methyl}amino)-1-(2-methylfuran-3-carbonyl)-1H-1,2,4-
triazol-3-yl]pyrrolidin-2-ol
4-[5-({[4-(aminomethyl)phenyl]methyl}amino)-1-(furan-2-carbonyl)-1H-1,2,4-triazol-3-
yl]-1-(2,2-dimethylpropanoyl)-3-(trifluoromethyl)piperidin-2-one
4-[5-({[4-(aminomethyl)phenyl]methyl}amino)-1-(furan-3-carbonyl)-1H-1,2,4-triazol-3-
yl]-1-(2,2-dimethylpropanoyl)-5-(trifluoromethyl)piperidin-3-one
4-[5-({[4-(aminomethyl)phenyl]methyl}amino)-1-(thiophene-3-carbonyl)-1H-1,2,4-
triazol-3-yl]-1-(3-hydroxypyrrolidine-1-carbonyl)-3-methylazetidin-2-one
4-[5-({[4-(aminomethyl)phenyl]methyl}sulfanyl)-1-(2-fluorobenzoyl)-1H-1,2,4-triazol-3-
yl]-1-(pyrrolidine-1-sulfonyl)-3-(trifluoromethyl)azetidin-2-one
4-[5-({[4-(aminomethyl)phenyl]methyl}sulfanyl)-1-(2-fluorobenzoyl)-1H-1,2,4-triazol-3-
yl]-1-[2-(morpholin-4-yl)-2-oxoethyl]-5-(trifluoromethyl)piperidin-3-one
4-[5-({[4-(aminomethyl)phenyl]methyl}sulfanyl)-1-(2-fluorobenzoyl)-1H-1,2,4-triazol-3-
yl]-1-methanesulfonyl-3-(trifluoromethyl)azetidin-2-one
4-[5-({[4-(aminomethyl)phenyl]methyl}sulfanyl)-1-(thiophene-2-carbonyl)-1H-1,2,4-
triazol-3-yl]-1-(pyrrolidine-1-sulfonyl)piperidin-3-one
4-[5-({[4-(aminomethyl)phenyl]methyl}sulfanyl)-1H-1,2,4-triazol-3-yl]-1-[(3-
hydroxypyrrolidin-1-yl)sulfonyl]-5-methylpyrrolidin-2-ol
4-[5-({[4-(aminomethyl)phenyl]methyl}sulfanyl)-1H-1,2,4-triazol-3-yl]-3-methyl-1-
(morpholine-4-carbonyl)piperidin-2-one
4-{1-benzoyl-5-[(4-fluorophenyl)methoxy]-1H-1,2,4-triazol-3-yl}-1-[2-(morpholin-4-
yl)acetyl]piperidin-2-one
4-{1-benzoyl-5-[(5-chlorothiophen-2-yl)methoxy]-1H-1,2,4-triazol-3-yl}-1-(pyrrolidine-1-
sulfonyl)piperidin-2-one
4-{1-benzoyl-5-[(5-chlorothiophen-2-yl)methoxy]-1H-1,2,4-triazol-3-yl}piperidine
4-{5-[(4-fluorophenyl)methoxy]-1-(2-methoxybenzoyl)-1H-1,2,4-triazol-3-yl}-1-(3-
hydroxypyrrolidine-1-carbonyl)-3-(trifluoromethyl)piperidin-2-one
4-{5-[(4-fluorophenyl)methoxy]-1-(2-methoxybenzoyl)-1H-1,2,4-triazol-3-yl}-1-[2-
(morpholin-4-yl)-2-oxoethyl]-5-(trifluoromethyl)pyrrolidin-3-one
4-{5-[(4-fluorophenyl)methoxy]-1H-1,2,4-triazol-3-yl}-1-(3-hydroxypyrrolidine-1-
carbonyl)-3-methylazetidin-2-one
4-{5-[(5-chlorothiophen-2-yl)methoxy]-1-(1,3-thiazole-4-carbonyl)-1H-1,2,4-triazol-3-
yl}-3-methyl-1-(pyrrolidine-1-carbonyl)piperidin-2-one
4-{5-[(5-chlorothiophen-2-yl)methoxy]-1-(1,3-thiazole-4-carbonyl)-1H-1,2,4-triazol-3-
yl}-3-methylpiperidin-2-one
4-{5-[(5-chlorothiophen-2-yl)methoxy]-1-(2-fluorobenzoyl)-1H-1,2,4-triazol-3-yl}-1-
(pyrrolidine-1-sulfonyl)-3-(trifluoromethyl)pyrrolidin-2-one
4-{[(1-benzoyl-3-{1-[2-(morpholin-4-yl)acetyl]piperidin-3-yl}-1H-1,2,4-triazol-5-
yl)amino]methyl}benzene-1-carboximidamide
4-{[(3-{1-[(3-hydroxypyrrolidin-1-yl)sulfonyl]-4-methyl-2-oxopyrrolidin-3-yl}-1-
(thiophene-3-carbonyl)-1H-1,2,4-triazol-5-yl)(methyl)amino]methyl}benzene-1-
carboximidamide
4-{[(3-{1-[(3-hydroxypyrrolidin-1-yl)sulfonyl]-4-oxopyrrolidin-3-yl}-1-(thiophene-2-
carbonyl)-1H-1,2,4-triazol-5-yl)amino]methyl}benzene-1-carboximidamide
4-{[(3-{1-[2-(morpholin-4-yl)acetyl]pyrrolidin-3-yl}-1-(thiophene-2-carbonyl)-1H-1,2,4-
triazol-5-yl)amino]methyl}benzene-1-carboximidamide
4-{[(3-{3-methyl-1-[2-(morpholin-4-yl)-2-oxoethyl]piperazin-2-yl}-1H-1,2,4-triazol-5-
yl)oxy]methyl}benzene-1-carboximidamide
4-{[(3-{4-methyl-1-[2-(morpholin-4-yl)-2-oxoethyl]-2-oxopiperidin-3-yl}-1-(1,3-thiazole-
4-carbonyl)-1H-1,2,4-triazol-5-yl)amino]methyl}benzene-1-carboximidamide
4-{[(3-{4-methyl-1-[2-(morpholin-4-yl)acetyl]piperidin-3-yl}-1H-1,2,4-triazol-5-
yl)amino]methyl}benzene-1-carboximidamide
4-{[(3-{5-hydroxy-1-[(3-hydroxypyrrolidin-1-yl)sulfonyl]-2-methylpyrrolidin-3-yl}-1-
(thiophene-3-carbonyl)-1H-1,2,4-triazol-5-yl)sulfanyl]methyl}benzene-1-
carboximidamide
4-{[methyl({3-[3-methyl-5-oxo-1-(pyrrolidine-1-carbonyl)piperidin-4-yl]-1-(4-
methylfuran-3-carbonyl)-1H-1,2,4-triazol-5-yl})amino]methyl}benzene-1-
carboximidamide
5-(5-{[(4-carbamimidoylphenyl)methyl]amino}-1H-1,2,4-triazol-3-yl)-N,N,4-trimethyl-2-
oxopiperidine-1-carboxamide
5-(5-{[(4-fluorophenyl)methyl](methyl)amino}-1-(2-methylfuran-3-carbonyl)-1H-1,2,4-
triazol-3-yl)-1-(morpholine-4-carbonyl)piperidin-2-one
5-(5-{[(4-fluorophenyl)methyl](methyl)amino}-1-(5-methylfuran-3-carbonyl)-1H-1,2,4-
triazol-3-yl)-1-(3-hydroxypyrrolidine-1-carbonyl)-4-(trifluoromethyl)piperidin-2-one
(5-(5-{[(4-fluorophenyl)methyl](methyl)amino}-1-(furan-3-carbonyl)-1H-1,2,4-triazol-3-
yl)-4-(trifluoromethyl)piperidin-2-one
5-(5-{[(4-fluorophenyl)methyl](methyl)amino}-1-(thiophene-3-carbonyl)-1H-1,2,4-
triazol-3-yl)-1-[(3-hydroxypyrrolidin-1-yl)sulfonyl]-4-methylpiperidin-2-one
5-(5-{[(4-fluorophenyl)methyl]amino}-1-(2-methylfuran-3-carbonyl)-1H-1,2,4-triazol-3-
yl)-1-(pyrrolidine-1-carbonyl)piperidin-2-one
5-(5-{[(4-fluorophenyl)methyl]amino}-1-(5-methylfuran-3-carbonyl)-1H-1,2,4-triazol-3-
yl)-1-(pyrrolidine-1-sulfonyl)-4-(trifluoromethyl)piperidin-2-one
5-(5-{[(4-fluorophenyl)methyl]amino}-1-(thiophene-3-carbonyl)-1H-1,2,4-triazol-3-yl)-1-
methanesulfonyl-4-methylpiperidin-2-one
5-(5-{[(5-chlorothiophen-2-yl)methyl](methyl)amino}-1-(2,2-dimethylpropanoyl)-1H-
1,2,4-triazol-3-yl)-4-(trifluoromethyl)piperidin-2-one
5-(5-{[(5-chlorothiophen-2-yl)methyl](methyl)amino}-1-(2-methoxybenzoyl)-1H-1,2,4-
triazol-3-yl)-1-(3-hydroxypyrrolidine-1-carbonyl)-4-(trifluoromethyl)piperidin-2-one
5-(5-{[(5-chlorothiophen-2-yl)methyl](methyl)amino}-1-(2-methoxybenzoyl)-1H-1,2,4-
triazol-3-yl)-1-(pyrrolidine-1-sulfonyl)-4-(trifluoromethyl)piperidin-2-one
5-(5-{[(5-chlorothiophen-2-yl)methyl](methyl)amino}-1H-1,2,4-triazol-3-yl)-1-
methanesulfonyl-4-methylpiperidin-2-one
5-(5-{[(5-chlorothiophen-2-yl)methyl]sulfanyl}-1-(4-methylfuran-3-carbonyl)-1H-1,2,4-
triazol-3-yl)-N,N,4-trimethyl-2-oxopiperidine-1-sulfonamide
5-(5-{[(5-chlorothiophen-2-yl)methyl]sulfanyl}-1-(thiophene-3-carbonyl)-1H-1,2,4-
triazol-3-yl)-N,N,4-trimethyl-2-oxopiperidine-1-carboxamide
5-[(4-fluorophenyl)methoxy]-3-(1-methanesulfonyl-2-methylazetidin-3-yl)-1-(1,3-
thiazole-4-carbonyl)-1H-1,2,4-triazole
5-[(5-chlorothiophen-2-yl)methoxy]-1-(2-fluorobenzoyl)-3-[3-(trifluoromethyl)azetidin-2-
yl]-1H-1,2,4-triazole
5-[(5-chlorothiophen-2-yl)methoxy]-3-(2-methyloxolan-3-yl)-1H-1,2,4-triazole
5-[(5-chlorothiophen-2-yl)methoxy]-3-(3-methyloxetan-2-yl)-1H-1,2,4-triazole
5-[5-({[4-(aminomethyl)phenyl]methyl}(methyl)amino)-1-(thiophene-2-carbonyl)-1H-
1,2,4-triazol-3-yl]-1-[2-(morpholin-4-yl)-2-oxoethyl]piperidin-2-one
5-{5-[(5-chlorothiophen-2-yl)methoxy]-1-(thiophene-2-carbonyl)-1H-1,2,4-triazol-3-yl}-
1-[2-(morpholin-4-yl)acetyl]piperidin-2-one
5-{5-[(5-chlorothiophen-2-yl)methoxy]-1H-1,2,4-triazol-3-yl}-1-[(3-hydroxypyrrolidin-1-
yl)sulfonyl]-4-methylpiperidin-2-one
5-{[(4-fluorophenyl)methyl]sulfanyl}-1-(2-methoxybenzoyl)-3-[1-(pyrrolidine-1-
carbonyl)-3-(trifluoromethyl)azetidin-2-yl]-1H-1,2,4-triazole
5-{[(4-fluorophenyl)methyl]sulfanyl}-3-(3-methyloxan-4-yl)-1H-1,2,4-triazole
5-{[(4-fluorophenyl)methyl]sulfanyl}-3-[1-(pyrrolidine-1-sulfonyl)azetidin-3-yl]-1-
(thiophene-2-carbonyl)-1H-1,2,4-triazole
5-{[(5-chlorothiophen-2-yl)methyl]sulfanyl}-1-(5-methylfuran-3-carbonyl)-3-[1-
(pyrrolidine-1-carbonyl)-2-(trifluoromethyl)azetidin-3-yl]-1H-1,2,4-triazole
N-[(4-fluorophenyl)methyl]-1-(furan-3-carbonyl)-3-[1-(morpholine-4-carbonyl)-3-
(trifluoromethyl)piperidin-4-yl]-1H-1,2,4-triazol-5-amine
N-[(4-fluorophenyl)methyl]-1-(furan-3-carbonyl)-3-[1-methanesulfonyl-3-
(trifluoromethyl)piperazin-2-yl]-N-methyl-1H-1,2,4-triazol-5-amine
N-[(4-fluorophenyl)methyl]-3-(1-methanesulfonyl-3-methylpiperidin-4-yl)-1-(4-
methylfuran-3-carbonyl)-1H-1,2,4-triazol-5-amine
N-[(4-fluorophenyl)methyl]-3-(3-methyloxan-4-yl)-1-(thiophene-3-carbonyl)-1H-1,2,4-
triazol-5-amine
N-[(5-chlorothiophen-2-yl)methyl]-1-(2-methylfuran-3-carbonyl)-3-(oxan-3-yl)-1H-1,2,4-
triazol-5-amine
N-[(5-chlorothiophen-2-yl)methyl]-1-(5-methylfuran-3-carbonyl)-3-[1-(pyrrolidine-1-
carbonyl)-3-(trifluoromethyl)azetidin-2-yl]-1H-1,2,4-triazol-5-amine
N-[(5-chlorothiophen-2-yl)methyl]-1-(5-methylfuran-3-carbonyl)-3-[1-(pyrrolidine-1-
carbonyl)-3-(trifluoromethyl)piperazin-2-yl]-1H-1,2,4-triazol-5-amine
N-[(5-chlorothiophen-2-yl)methyl]-1-(5-methylfuran-3-carbonyl)-3-[4-(pyrrolidine-1-
carbonyl)-3-(trifluoromethyl)piperazin-2-yl]-1H-1,2,4-triazol-5-amine
N-[(5-chlorothiophen-2-yl)methyl]-1-(furan-3-carbonyl)-3-[1-(pyrrolidine-1-carbonyl)-3-
(trifluoromethyl)piperidin-4-yl]-1H-1,2,4-triazol-5-amine
N-[(5-chlorothiophen-2-yl)methyl]-1-(furan-3-carbonyl)-3-[4-methanesulfonyl-3-
(trifluoromethyl)piperazin-2-yl]-1H-1,2,4-triazol-5-amine
N-[(5-chlorothiophen-2-yl)methyl]-3-(1-methanesulfonyl-3-methylpiperidin-4-yl)-N-
methyl-1-(1,3-thiazole-4-carbonyl)-1H-1,2,4-triazol-5-amine
N-[(5-chlorothiophen-2-yl)methyl]-3-[2-methyl-1-(morpholine-4-carbonyl)piperidin-3-yl]-
1-(4-methylfuran-3-carbonyl)-1H-1,2,4-triazol-5-amine
N-[(5-chlorothiophen-2-yl)methyl]-N-methyl-3-[1-(morpholine-4-carbonyl)pyrrolidin-3-
yl]-1-(thiophene-2-carbonyl)-1H-1,2,4-triazol-5-amine
N-{[4-(aminomethyl)phenyl]methyl}-1-(2-fluorobenzoyl)-N-methyl-3-[3-
(trifluoromethyl)morpholin-2-yl]-1H-1,2,4-triazol-5-amine
N-{[4-(aminomethyl)phenyl]methyl}-1-(4-methylfuran-3-carbonyl)-3-(4-methylpiperidin-
3-yl)-1H-1,2,4-triazol-5-amine
N-{[4-(aminomethyl)phenyl]methyl}-1-(furan-2-carbonyl)-3-[3-
(trifluoromethyl)piperazin-2-yl]-1H-1,2,4-triazol-5-amine
N-{[4-(aminomethyl)phenyl]methyl}-1-benzoyl-N-methyl-3-[1-(morpholine-4-
carbonyl)piperazin-2-yl]-1H-1,2,4-triazol-5-amine
N-{[4-(aminomethyl)phenyl]methyl}-3-(1-methanesulfonyl-2-methylazetidin-3-yl)-1-(4-
methylfuran-3-carbonyl)-1H-1,2,4-triazol-5-amine
N-{[4-(aminomethyl)phenyl]methyl}-3-(1-methanesulfonyl-4-methylpyrrolidin-3-yl)-N-
methyl-1H-1,2,4-triazol-5-amine
N-{[4-(aminomethyl)phenyl]methyl}-3-(4-methylpyrrolidin-3-yl)-1-(thiophene-3-
carbonyl)-1H-1,2,4-triazol-5-amine
[4-({[1-(2-chlorobenzoyl)-3-[1-(morpholine-4-carbonyl)piperazin-2-yl]-1H-1,2,4-triazol-
5-yl]oxy}methyl)phenyl]methanamine
[4-({[1-(2-methoxybenzoyl)-3-[1-(pyrrolidine-1-carbonyl)-2-(trifluoromethyl)azetidin-3-
yl]-1H-1,2,4-triazol-5-yl]sulfanyl}methyl)phenyl]methanamine
[4-({[1-(2-methoxybenzoyl)-3-[2-(trifluoromethyl)oxetan-3-yl]-1H-1,2,4-triazol-5-
yl]sulfanyl}methyl)phenyl]methanamine
[4-({[1-(furan-2-carbonyl)-3-[3-(trifluoromethyl)morpholin-2-yl]-1H-1,2,4-triazol-5-
yl]oxy}methyl)phenyl]methanamine
[4-({[1-(furan-2-carbonyl)-3-[4-(morpholine-4-carbonyl)-3-(trifluoromethyl)piperazin-2-
yl]-1H-1,2,4-triazol-5-yl]oxy}methyl)phenyl]methanamine
[4-({[1-benzoyl-3-(morpholin-3-yl)-1H-1,2,4-triazol-5-
yl]sulfanyl}methyl)phenyl]methanamine
[4-({[3-(1-methanesulfonyl-2-methylpiperidin-3-yl)-1-(1,3-thiazole-4-carbonyl)-1H-1,2,4-
triazol-5-yl]sulfanyl}methyl)phenyl]methanamine
[4-({[3-(1-methanesulfonyl-4-methylpyrrolidin-3-yl)-1-(thiophene-3-carbonyl)-1H-1,2,4-
triazol-5-yl]oxy}methyl)phenyl]methanamine
{4-[({3-[4-methyl-1-(pyrrolidine-1-carbonyl)pyrrolidin-3-yl]-1-(1,3-thiazole-4-carbonyl)-
1H-1,2,4-triazol-5-yl}sulfanyl)methyl]phenyl}methanamine

Compounds disclosed herein also include racemic mixtures, stereoisomers and mixtures of the compounds, including isotopically-labeled and radio-labeled compounds. See e.g., Goding, 1986, MONOCLONAL ANTIBODIES PRINCIPLES AND PRACTICE; Academic Press, p. 104. Such isomers can be isolated by standard resolution techniques, including e.g., fractional crystallization, chiral chromatography, and the like. See e.g., Eliel, E. L. & Wilen S. H., 1993, STEREOCHEMISTRY IN ORGANIC COMPOUNDS; John Wiley & Sons, New York.

In some embodiments, compounds disclosed herein have asymmetric centers and can occur as racemates, racemic mixtures, and as individual enantiomers or diastereoisomers, with all isomeric forms as well as mixtures thereof being contemplated for use in the compounds and methods described herein. The compounds contemplated for use in the compounds and methods described herein do not include those that are known in the art to be too unstable to synthesize and/or isolate.

The compounds disclosed herein can also contain unnatural proportions of atomic isotopes at one or more of the atoms that constitute such compounds. For example, the compounds can be radiolabeled with radioactive isotopes, such as for example tritium (³H), iodine-125 (¹²⁵I), or carbon-14 (¹⁴C). All isotopic variations of the compounds disclosed herein, whether radioactive or not, are encompassed within the contemplated scope.

In some embodiments, metabolites of the compounds disclosed herein are useful for the methods disclosed herein.

In some embodiments, compounds contemplated herein are provided in the form of a prodrug. The term “prodrug” refers to a compound that can be converted into a compound (e.g., a biologically active compound) described herein in vivo. Prodrugs can be useful for a variety of reason known in the art, including e.g., ease of administration due e.g., to enhanced bioavailability in oral administration, and the like. The prodrug can also have improved solubility in pharmaceutical compositions over the biologically active compounds. An example, without limitation, of a prodrug is a compound which is administered as an ester (i.e., the “prodrug”) to facilitate transmittal across a cell membrane where water solubility is detrimental to mobility but which then is metabolically hydrolyzed to the carboxylic acid, the active entity, once inside the cell where water solubility is beneficial. Conventional procedures for the selection and preparation of suitable prodrug derivatives are described, for example, in DESIGN OF PRODRUGS, (ed. H. Bundgaard, Elsevier, 1985), which is hereby incorporated herein by reference for the limited purpose describing procedures and preparation of suitable prodrug derivatives.

Accordingly, in some embodiments, compounds contemplated herein are provided in the form of a prodrug ester. The term “prodrug ester” refers to derivatives of the compounds disclosed herein formed by the addition of any of a variety of ester-forming groups, e.g., groups known in the art, that are hydrolyzed under physiological conditions. Examples of prodrug ester groups include pivaloyloxymethyl, acetoxymethyl, phthalidyl, indanyl and methoxymethyl, as well as other such groups known in the art, including a (5-R-2-oxo-1,3-dioxolen-4-yl)methyl group. Other examples of prodrug ester groups can be found in, for example, T. Higuchi and V. Stella, in “Pro-drugs as Novel Delivery Systems”, Vol. 14, A.C.S. Symposium Series, American Chemical Society (1975); and BIOREVERSIBLE CARRIERS IN DRUG DESIGN: THEORY AND APPLICATION, edited by E. B. Roche, Pergamon Press: New York, 14-21 (1987) (providing examples of esters useful as prodrugs for compounds containing carboxyl groups). Each of the above-mentioned references is herein incorporated by reference for the limited purpose of disclosing ester-forming groups that can form prodrug esters.

In some embodiments, prodrugs can be slowly converted to the compounds described herein useful for the methods described herein when placed in a transdermal patch reservoir with a suitable enzyme or chemical reagent.

Certain compounds disclosed herein can exist in unsolvated forms as well as solvated forms, including hydrated forms. In general, the solvated forms are equivalent to unsolvated forms and are encompassed within the scope of contemplated compounds. Certain compounds of the present invention can exist in multiple crystalline or amorphous forms. In general, all physical forms are equivalent for the compounds and methods contemplated herein and are intended to be within the scope disclosed herein.

### III. Biological Activities

In some embodiments, compounds described herein exhibit inhibitory activity against thrombin with activities≧1 μM, e.g., about 1, 2, 3, 4, 5, 6, 7, 8, 9, 10, 11, 12, 13, 14, 16, 18, 20, 22, 24, 26, 28, 30, 32, 34, 36, 38, 40, 45, 50, 55, 60, 65, 70, 75, 80, 85, 90, 95, 100 μM, or even greater. In some embodiments, the compounds exhibit inhibitory activity against thrombin with activities between 0.1 μM and 1 μM, e.g., about 0.1, 0.2, 0.3, 0.4, 0.5, 0.6, 0.7, 0.8, 0.9 or 1.0 μM. In some embodiments, compounds described herein exhibit inhibitory activity against thrombin with activities≦0.1 μM, e.g., about 1, 2, 5, 10, 15, 20, 30, 40, 50, 60, 70, 80, 90, or 100 nM. Ranges of values using a combination of any of the values recited herein as upper and/or lower limits are also contemplated, for example, but not limited to, 1-10 nM, 10-100 nM, 0.1-1 μM, 1-10 μM, 10-100 μM, 100-200 μM, 200-500 μM, or even 500-1000 μM. In some embodiments, the inhibitory activity is in the range of about 1-10 nM, 10-100 nM, 0.1-1 μM, 1-10 μM, 10-100 μM, 100-200 μM, 200-500 μM, or even 500-1000 μM. It is understood that for purposes of quantification, the terms “activity,” “inhibitory activity,” “biological activity,” “thrombin activity and the like in the context of an inhibitory compound disclosed herein can be quantified in a variety of ways known in the art. Unless indicated otherwise, as used herein such terms refer to IC₅₀ in the customary sense (i.e., concentration to achieve half-maximal inhibition).

Inhibitory activity against thrombin in turn inhibits the blood coagulation process. Accordingly, compounds disclosed herein are indicated in the treatment or management of thrombotic disorders. In some embodiments, a dose or a therapeutically effective dose of a compound disclosed herein will be that which is sufficient to achieve a plasma concentration of the compound or its active metabolite(s) within a range set forth herein, e.g., about 1-10 nM, 10-100 nM, 0.1-1 μM, 1-10 μM, 10-100 μM, 100-200 μM, 200-500 μM, or even 500-1000 μM, preferably about 1-10 nM, 10-100 nM, or 0.1-1 μM. Without wishing to be bound by any theory, it is believe that such compounds are indicated in the treatment or management of thrombotic disorders.

In some embodiments, compounds described herein exhibit inhibitory activity against KLKB1 with activities between 1 μM and 10 μM, e.g., about 1, 2, 3, 4, 5, 6, 7, 8, 9 or 10 μM. In some embodiments, compounds described herein exhibit inhibitory activity against KLKB1 with activities 10 μM, e.g., about 10, 20, 50, 100, 150, 200, 300, 400, 500, 600, 700, 800, 900, 1000 μM or even greater. In some embodiments, compounds described herein exhibit inhibitory activity against KLKB1 with activities 1 μM, e.g., about 900, 800, 700, 600, 500, 400, 300, 200, 100, 50 nM or even lower. Ranges of values using a combination of any of the values recited herein as upper and/or lower limits are also contemplated, for example, but not limited to, 1-10 nM, 10-100 nM, 0.1-1 μM, 1-10 μM, 10-100 μM, 100-200 μM, 200-500 μM, or even 500-1000 μM. In some embodiments, the inhibitory activity is in the range of about 1-10 nM, 10-100 nM, 0.1-1 μM, 1-10 μM, 10-100 μM, 100-200 μM, 200-500 μM, or even 500-1000 μM. It is understood that for purposes of quantification, the terms “activity,” “inhibitory activity,” “biological activity,” “KLKB1 activity” and the like in the context of an inhibitory compound disclosed herein can be quantified in a variety of ways known in the art. Unless indicated otherwise, as used herein such terms refer to IC₅₀ in the customary sense (i.e., concentration to achieve half-maximal inhibition).

Inhibitory activity against KLKB1 has an effect on the coagulation cascade and the inflammatory response. Thus, it has been proposed that KLKB1 inhibitors can be useful in the treatment of thrombotic and fibrinolytic diseases and disease conditions.

Accordingly, compounds disclosed herein are indicated in the treatment or management of a variety of diseases or disorders. In some embodiments, a dose or a therapeutically effective dose of a compound disclosed herein will be that which is sufficient to achieve a plasma concentration of the compound or its active metabolite(s) within a range set forth herein, e.g., about 1-10 nM, 10-100 nM, 0.1-1 μM, 1-10 μM, 10-100 μM, 100-200 μM, 200-500 μM, or even 500-1000 μM, preferably about 1-10 nM, 10-100 nM, or 0.1-1 μM. Without wishing to be bound by any theory, it is believe that such compounds are indicated in the treatment or management of diseases associated with thrombin or kallikrein.

### IV. Methods of Treating and Preventing Disease

Thrombin-Related Diseases and Conditions (e.g. Thrombosis).

Thrombotic diseases are the primary indications for thrombin inhibition, because of thrombin's location in the coagulation cascade and, in turn, the importance of the coagulation cascade in the progression of blood clotting processes. However, without wishing to be bound by any theory, it is believed the coagulation cascade in general, and thrombin in particular, is important in a variety other disease states.

It has been discovered that compounds described herein, e.g., multisubstituted aromatic compounds, exhibit inhibitory action against thrombin (activated blood-coagulation factor II; EC 3.4.21.5). This, in turn inhibits the blood coagulation process.

This inhibitory action is useful in the treatment of a variety of thrombotic disorders, such as, but not limited to, acute vascular diseases such as acute coronary syndromes; venous-, arterial- and cardiogenic thromboembolisms; the prevention of other states such as disseminated intravascular coagulation, or other conditions that involve the presence or the potential formation of a blood clot thrombus. Other indications for methods described herein include the following.

It has long been recognized that cancer progression is accompanied by venous thrombosis, but it has not been understood how each disease is related. From several clinical trials studying the treatment of VTE, meta-analyses have shown that low molecular weight heparins (LMWHs) improve overall survival in subgroups of cancer patients. See e.g., Zacharski, L. R. & Lee, A. Y., 2008, Expert Opin Investig Drugs, 17:1029-1037; Falanga, A. & Piccioli, A., 2005, Current Opinion in Pulmonary Medicine, 11:403-407; Smorenburg, S. M., et al., 1999, Thromb Haemost, 82:1600-1604; Hettiarachchi, R. J., et al., 1999, Thromb Haemost, 82:947-952. This finding was substantiated in later clinical trials that measured specifically the survival of cancer patients. See e.g., Lee, A. Y. et al., 2005, J Clin Oncol, 23:2123-2129; Klerk, C. P. et al., J Clin Oncol 2005, 23:2130-2135; Kakkar, A. K., et al., 2004, J Clin Oncol, 22:1944-1948; Altinbas, M., et al., 2004, J Thromb Haemost, 2:1266-1271.

More recently, researchers have focused on the specific anticancer effect of DTIs. For example, it was shown that heparin significantly prolonged the survival of patients with limited small cell lung cancer. See e.g., Akl, E. A., et al., 2008, J Exp Clin Cancer Res, 27:4. Other investigators found that systemic use of argatroban reduced tumor mass and prolonged survival time in rat glioma models leading to the conclusion that argatroban should be considered as a novel therapeutic for glioma, a notoriously difficult to treat cancer type. See e.g., Hua, Y., et al., 2005, Acta Neurochir, Suppl 2005, 95:403-406; Hua, Y., et al., 2005, J Thromb Haemost, 3:1917-1923. Very recently, it was demonstrated that dabigatran etexilate, a DTI recently FDA-approved (see e.g., Hughes, B., 2010, Nat Rev Drug Discov, 9:903-906) for DVT indications, inhibited both the invasion and metastasis of malignant breast tumors. See e.g., DeFeo, K. et al., 2010, Thrombosis Research, 125 (Supplement 2): S188-S188; Defeo, K., et al., 2010, Cancer Biol Ther, 10:1001-1008. Thus, dabigatran etexilate treatment led to a 50% reduction in tumor volume at 4 weeks with no weight loss in treated mice. Dabigatran etexilate also reduced tumor cells in the blood and liver micrometastases by 50-60%. These investigators concluded that dabigatran etexilate can be beneficial in not only preventing thrombotic events in cancer patients, but also as adjunct therapy to treat malignant tumors.

Further, hirudin and the LMWH nadroparin dramatically reduced the number of lung metastases when administered prior to cancer cell inoculation. See e.g., Hu, L., et al., 2004, Blood, 104:2746-51.

The de novo thrombin inhibitor d-Arg-Oic-Pro-d-Ala-Phe(p-Me) has been found to block thrombin-stimulated invasion of prostate cancer cell line PC-3 in a concentration dependent manner. See e.g., Nieman, M. T., et al., 2008, J Thromb Haemost, 6:837-845. A reduced rate of tumor growth was observed in mice dosed with the pentapeptide through their drinking water. The mice also showed reduced fold rate in tumor size and reduced overall tumor weight compared to untreated mice. Microscopic examination of treated tumors showed reduced number of large blood vessels thus concluding that the pentapeptide interfered with tumor angiogenesis. Nieman, M. T., et al., Thromb Haemost, 104:1044-8.

In view of these and related studies, it is suggested that anticoagulants affect tumor metastasis; that is, angiogenesis, cancer cell adhesion, migration and invasion processes. See e.g., Van Noorden, C. J., et al., 2010, Thromb Res, 125 Suppl 2:S77-79.

Alzheimer's Disease.

Very recent experiments confirm higher thrombin levels in brain endothelial cells of patients with Alzheimer's disease. While ‘normal’ thrombin levels are connected to regulatory CNS functions, thrombin accumulation in the brain is toxic. It has also been found that the neural thrombin inhibitor Protease Nexin 1 (PN-1) is significantly reduced in the Alzheimer's disease brain, despite the fact that PN-1 mRNA levels are unchanged. These observations have led some investigators to suggest that reduction of CNS-resident thrombin will prove useful in Alzheimer's Disease (AD) treatment. See e.g., Vaughan, P. J., et al., 1994, Brain Res, 668:160-170; Yin, X., et al., 2010, Am J Pathol, 176:1600-1606; Akiyama, H., et al., 1992, Neurosci Lett, 146:152-154.

Multiple Sclerosis.

Investigators found that hirudin treatment in an animal model of Multiple Sclerosis (MS) showed a dramatic improvement in disease severity. See e.g., Han, M. H., et al., 2008, Nature, 451:1076-1081. Similar results were obtained following treatment with heparin (a DTI) and dermatan sulfate, another coagulation inhibitor. See e.g., Chelmicka-Szorc, E. & Amason, B. G., 1972, Arch Neurol, 27:153-158; Inaba, Y., et al., 1999, Cell Immunol, 198:96-102. Other evidence shows that naturally occurring antithrombin III has anti-inflammatory effects in diseases such as endotoxemia and other sepsis-related conditions. See e.g., Wiedermann, C. J. & Romisch, J., 2002, Acta Med Austriaca, 29:89-92. Naturally occurring thrombin inhibitors are presumably synthesized in situ and have protective roles in CNS inflammation. Therefore, therapeutic thrombin inhibition has been proposed as a potential MS treatment. See e.g., Luo, W., et al., 2009, In: THROMBIN, Maragoudakis, M. E.; Tsopanoglou, N. E., Eds. Springer New York: 2009; pp 133-159.

Pain.

In a rat pain model with partial lesion of the sciatic nerve, intrathecal hirudin prevented the development of neuropathic pain and curbed pain responses for 7 days. The investigators found that following injury, neuropathic pain was mediated by thrombin generation, which in turn activated PAR-1 receptor in the spinal cord. Hirudin inhibited thrombin generation and ultimately led to pain relief. See e.g., Garcia, P. S., et al., 2010, Thromb Haemost, 103:1145-1151; Narita, M., et al., 2005, J Neurosci, 25:10000-10009. Researchers hypothesize that thrombin and the PARs are involved not just as part of the coagulation cascade, but in inflammation, nociception and neurodevelopment. Development of a DTI to intersect an unexploited pharmacology will lead to pain therapeutics distinct from opioids and NSAIDs, whose shortcomings are well documented. See e.g., Garcia 2010, Id. Known thrombin inhibitors have been reported to be useful in preventing stroke in individuals with atrial fibrillation. The selective thrombin inhibitor ximelagatran was studied in two phase III clinical trials ((SPORTIF III and SPORTIF V), which compared ximelagatran to warfarin for the prevention of cardioembolic events in patients with non-valvular atrial fibrillation. The investigators for the SPORTIF III clinical trial that ximelagatran, administered in a fixed dose without coagulation monitoring, protects high-risk patients with atrial fibrillation against thromboembolism at least as effectively as well-controlled warfarin, and is associated with less bleeding. When the results of SPORTIF III and V were combined, ximelagatran was associated with a 16% relative risk reduction in the composite outcome measure of all strokes (ischemic or hemorrhagic), systemic embolic events, major bleeding, and death. (Olsson, S. B. Lancet 2003, 362 (9397), 1691-1698; Hirsh, J. et al. Blood 2005, 105 (2), 453-463; Clemens, A. et al. WIPO Patent Application WO/2008/009638). Without further wishing to be bound by any theory, it is reasonable to believe that thrombin inhibition in general can be useful in preventing stroke in individuals with atrial fibrillation.

Known thrombin inhibitors have been reported to be useful in the treatment and prevention of acute coronary syndrome (Clemens, A. et al. WIPO Patent Application WO/2008/009638). ACS is a group of symptoms that are caused by myocardial ischemia. The drug could be used as a prophylaxis for myocardial infarction, or a certain time after the event (e.g. after myocardial infarction, post-MI; i.e. chronic therapy, secondary prevention). Without further wishing to be bound by any theory, it is reasonable to believe that thrombin inhibition in general can be useful in treating and preventing acute coronary syndrome.

Known thrombin inhibitors have been reported to be useful in the prevention of recurrent cardiac events after myocardial infarction. The selective thrombin inhibitor ximelagatran was studied in a phase II clinical trial entitled ESTEEM, measuring the efficacy and safety of the oral direct thrombin inhibitor ximelagatran in patients with recent myocardial damage. The result of the ESTEEM trial supports the notion that long-term treatment with an oral direct thrombin inhibitor reduces arterial thrombotic events. Oral ximelagatran in combination with acetylsalicylic acid was more effective than acetylsalicylic acid alone in reducing the frequency of major cardiovascular events during 6 months of treatment in patients with a recent myocardial infarction. (Hirsh, J. et al. Blood 2005, 105 (2), 453-463.). Without further wishing to be bound by any theory, it is reasonable to believe that thrombin inhibition in general can be useful in preventing recurrent cardiac events after myocardial infarction.

Known thrombin inhibitors have been reported to be useful in post-operative prophylaxis of deep vein thrombosis. The selective thrombin inhibitor ximelagatran was found to be efficacious for the prevention of venous thromboembolism after following a medical procedure like total hip or knee replacement (Francis, C. W. et al. Ann Intern Med 2002; 137:648-55; Heit, J. A. et al. Arch Intern Med 2001; 161: 2215-21; Eriksson B I et al. Thromb Haemost 2003; 89: 288-96). Without further wishing to be bound by any theory, it is reasonable to believe that thrombin inhibition in general can be useful in post-operative prophylaxis of deep vein thrombosis.

Known thrombin inhibitors like dabigatran have been reported to be useful in long-term treatment of pulmonary embolism. (Robertson L, Kesteven P, McCaslin J E. Cochrane Database Syst Rev. 2015 Dec. 4; 12). Without further wishing to be bound by any theory, it is reasonable to believe that thrombin inhibition in general can be useful in treating pulmonary embolism.

Known thrombin inhibitors have been reported to be useful for the prevention of coagulation in patients undergoing percutaneous coronary intervention. Percutaneous coronary intervention (PCI) requires aggressive anticoagulation therapy, and was historically achieved with unfractionated heparin. However, in many patients heparin is contraindicated, especially in patients with heparin-induced thrombocytopenia (HIT). In such instances, the endovascular disruption and the hypercoagulable state that characterized HIT means patients are put at risk of thrombosis during PCI. (Lewis, B. E. et al. Catheterization and cardiovascular interventions 2002, 57 (2), 177-184; Kokolis, S et al. Progress in cardiovascular diseases 2004, 46 (6), 506-523.) Dabigatran, which had already been claimed as a thrombin inhibitor and a useful anticoagulant in the clinical setting, was also published as a secondary medication in percutaneous interventional cardiac catherization. (Reilly et al. WIPO Patent Application WO/2010/020602). Without further wishing to be bound by any theory, it is reasonable to believe that thrombin inhibition in general can be useful in preventing coagulation in patients undergoing percutaneous coronary intervention.

Known thrombin inhibitors have been reported to be useful for the treatment of pulmonary-arterial hypertension. Dabigatran, a selective thrombin inhibitor, has been published as a useful drug for the treatment of pulmonary-arterial hypertension (PAH). Furthermore, dabigatran had found use as a treatment of: (i); pulmonary hypertension caused by left heart disorders, (ii); pulmonary hypertension associated with lung diseases such as pulmonary fibroses, particularly idiopathic pulmonary fibrosis, and/or hypoxia, (iii); pulmonary hypertension caused by chronic thromboembolic diseases. (Feuring, M. WIPO Patent Application WO/2010/020600). Without further wishing to be bound by any theory, it is reasonable to believe that thrombin inhibition in general can be useful for the treatment of pulmonary-arterial hypertension.

Known thrombin inhibitors have been reported to be useful for the treatment of pulmonary-arterial hypertension caused by left heart disorders (Feuring, M. WIPO Patent Application WO/2010/020600). Without further wishing to be bound by any theory, it is reasonable to believe that thrombin inhibition in general can be useful for the treatment of pulmonary-arterial hypertension caused by left heart disorders.

Known thrombin inhibitors have been reported to be useful for the treatment of pulmonary-arterial hypertension associated with lung diseases such as pulmonary fibroses, particularly idiopathic pulmonary fibrosis, and/or hypoxia (Feuring, M. WIPO Patent Application WO/2010/020600). Without further wishing to be bound by any theory, it is reasonable to believe that thrombin inhibition in general can be useful for the treatment of pulmonary-arterial hypertension associated with lung diseases.

Known thrombin inhibitors have been reported to be useful for the treatment of pulmonary hypertension caused by chronic thromboembolic diseases (Feuring, M. WIPO Patent Application WO/2010/020600). Without further wishing to be bound by any theory, it is reasonable to believe that thrombin inhibition in general can be useful for the treatment of pulmonary hypertension caused by chronic thromboembolic diseases.

Non-valvular atrial fibrillation is a sustained cardiac disturbance often associated with heart disease. Known thrombin inhibitors like ximelagatran have been reported to be useful for stroke prevention in patients with non-valvular atrial fibrillation (Diener H.-C. Cerebrovasc Dis 2006; 21:279-293). Without further wishing to be bound by any theory, it is reasonable to believe that thrombin inhibition in general can be useful for stroke prevention in patients with non-valvular atrial fibrillation.

A Transient Ischemic Attack (TIA) is an acute episode of temporary neurologic dysfunction that typically lasts less than an hour; results from focal cerebral, spinal cord, or retinal ischemia; and is not associated with acute tissue infarction. In people who have a TIA, the incidence of subsequent stroke is as high as 11% over the next 7 days and 24-29% over the following 5 years. In view of the high short-term risk of stroke after TIA, many physicians believe antithrombotic therapy should be initiated as soon as intracranial hemorrhage has been ruled out. Stroke prevention medication typically recommended for cardioembolic TIA is as follows: For patients with atrial fibrillation after TIA, long-term anticoagulation with warfarin (aspirin 325 mg/day for those unable to take oral anticoagulants); In acute myocardial infarction (MI) with left ventricular thrombus, oral anticoagulation with warfarin; concurrent aspirin up to 162 mg/day for ischemic coronary artery disease [CAD]); In dilated cardiomyopathy, oral anticoagulation with warfarin or antiplatelet therapy; In rheumatic mitral valve disease, oral anticoagulation with warfarin. For patients with TIA and ischaemic stroke of cardiac origin due to atrial fibrillation, vitamin K antagonists (VKAs) are highly effective in preventing recurrent ischaemic stroke but have important limitations and are thus underused. Antiplatelet therapy is much less effective than VKAs. The direct thrombin inhibitor, dabigatran etexilate, has shown efficacy over warfarin in a recent trial. Other new anticoagulants, including the oral factor Xa inhibitors, rivaroxaban, apixaban, and edoxaban, the parenteral factor Xa inhibitor, idrabiotaparinux, and the novel VKA, tecarfarin, were being assessed in 2010. (Hankey, G. J.; Eikelboom, J. W. ‘Antithrombotic Drugs for Patients with Ischaemic Stroke and Transient Ischaemic Attack to Prevent Recurrent Major Vascular Events.’ The Lancet Neurology 2010, 9 (3), 273-284.)

Known thrombin inhibitors have been reported to be useful for the treatment of venous thromboembolism due to formation of a thrombus within a vein (venous thrombosis) associated with acquired (prolonged bedrest, surgery, injury, malignancy, pregnancy and postpartum states) or inherited (deficiency of natural coagulation inhibitors) risk factors (Marsic, L. P. et al. WIPO Patent Application WO/2003/048155). Without further wishing to be bound by any theory, it is reasonable to believe that thrombin inhibition in general can be useful for the treatment of venous thromboembolism due to formation of a thrombus within a vein associated with acquired or inherited risk factors and/or embolism of peripheral veins caused by a detached thrombus. An example of an acquired risk factor would be a previous venous thromboembolism.

Known thrombin inhibitors have been reported to be useful for the treatment of cardiogenic thromboembolism due to formation of a thrombus in the heart associated with cardiac arrhythmia, heart valve defect, prosthetic heart valves or heart disease, embolism of peripheral arteries caused by a detached thrombus, most commonly in the brain (ischemic stroke). See Marsic, L. P. et al. WIPO Patent Application WO/2003/048155. Without further wishing to be bound by any theory, it is reasonable to believe that thrombin inhibition in general can be useful for the treatment of cardiogenic thromboembolism.

Known thrombin inhibitors have been reported to be useful for the treatment of arterial thrombosis due to underlying atherosclerotic processes in the arteries which obstructs or occludes an artery and causes myocardial ischemia (angina pectoris, acute coronary syndrome) or myocardial infarction, obstructs or occludes a peripheral artery (ischemic peripheral artery disease) and obstructs or occludes the artery after the procedure on the blood vessel (reocclusion or restenosis after transluminal coronary angioplasty, reocclusion or restenosis after percutaneous transluminal angioplasty of peripheral arteries). See Marsic, L. P. et al. WIPO Patent Application WO/2003/048155. Without further wishing to be bound by any theory, it is reasonable to believe that thrombin inhibition in general can be useful for the treatment of arterial thrombosis.

Known thrombin inhibitors have been reported to be useful for the treatment of disseminated intravascular coagulation in a number of states (e.g., in complications in pregnancy, in metastasing malignant diseases, after extensive injuries, in bacterial sepsis) when thrombogenic activation causes dysfunctional coagulation with widespread formation of thrombi within the vascular system. See Marsic, L. P. et al. WIPO Patent Application WO/2003/048155. Without further wishing to be bound by any theory, it is reasonable to believe that thrombin inhibition in general can be useful for the treatment of disseminated intravascular coagulation.

Known thrombin inhibitors have been reported to be useful as an adjunct therapy in conjunction with thrombolytic therapy in recent myocardial infarction, in combination with aspirin in patients with unstable angina pectoris designed to undergo percutaneous transluminal angioplasty and in the treatment of patients with thrombosis and with heparin-induced thrombocytopenia (Marsic, L. P. et al. WIPO Patent Application WO/2003/048155). Without further wishing to be bound by any theory, it is reasonable to believe that thrombin inhibition in general can be useful as an adjunct therapy with other antithrombotic therapies.

Known thrombin inhibitors have been reported to be useful for the treatment of inflammation (Kirk, I. WIPO Patent Application WO/2000/041716), type I diabetes mellitus (Korsgren, O.; Nillson, B. WIPO Patent Application WO/2003/061682), cancer (Kakkar, A. K. et al. J Clin Oncol 2004, 22, (10), 1944-8; Hua, Y. et al. Acta Neurochir Suppl 2005, 95, 403-6; Nieman, M. T. et al. J Thromb Haemost, 6 (2008), 837-845; Van Ryn, J.; Clemens, A. WIPO Patent Application WO/2010/020601), fibrosis (Duplantier, J. G. et al. Gut, 2004, 53:1682-1687; Seijo, S. et al. J Hepatol, 2007, 46:286-294; Assy, N. et al. Dig Dis Sci, 2007, 52:1187-1193; Bogatkevich, G. S. et al. Arthritis Rheum, 2009, 60:3455-3464), and pain (Garcia, P. S. et al. Thromb Haemost, 103:1145-1151; Narita, M. et al. J Neurosci, 2005, 25:10000-10009). Metaanalyses of clinical trials that studied the use of anticoagulants in oncology patients showed that low molecular weight heparins (LMWHs), selective thrombin inhibitors, improve overall survival in subgroups of cancer patients. This finding was substantiated in later clinical trials, in particular the FAMOUS clinical trials, that measured specifically the survival of cancer patients.

Without further wishing to be bound by any theory, it is reasonable to believe that thrombin inhibition in general can be useful for the treatment of thrombotic diseases or disorders and/or diseases or disorders which involve a blood clot thrombus or the potential formation of a blood clot thrombus and/or further involves stroke and/or one or more transient ischemic attacks (TIA) and/or pulmonary hypertension. Such conditions include, for example, acute coronary syndrome, thromboembolism, thrombosis, inflammation, diabetes mellitus, cancer, fibrosis, Alzheimer's Disease, multiple sclerosis, pain, recurrent cardiac events after myocardial infarction, or the like.

Kallikrein-Related Diseases and Conditions.

Kallikrein-related diseases or disorders are biological conditions associated with or moderated by kallikrein. They include, but are not limited by, those conditions associated with biological pathways that are moderated by plasma kallikrein. An example of such a pathway is the kallikrein-kinin system (Moreau, M. E. 2005, Journal of Pharmacological Sciences, 99, 6). Kallikrein-related diseases or disorders include, but are not limited to, fibrosis, inflammation, thrombosis, hereditary angioedema, skin disorders, cancer, and ophthalmic diseases. Ophthalmic diseases include, but are not limited to, diabetic macular edema, diabetic retinopathy, and age-related macular degeneration.

Diabetic Macular Edema.

In rodent models, it has been shown that activation of KLKB1 in the eye increases retinal vascular permeability; whereas inhibition of the kallikrein-kinin system reduces retinal leakage induced by diabetes and hypertension. These findings suggest that intraocular activation of the KLKB1 pathway can contribute to excessive retinal vascular permeability that can lead to diabetic macular edema. Thus, evidence suggests that KLKB1 inhibitors can provide a new therapeutic opportunity to reduce retinal vascular permeability (Feener, E. P. 2010, Curr Diab Rep 10, 270).

Hereditary Angioedema.

Ecallantide (Kalbitor) is a 60-amino acid recombinant protein that acts as a potent reversible inhibitor of KLKB1 (Schneider L, et al. 2007, J Allergy Clin Immunol, 120, 416) and has been approved by the FDA for the treatment of acute attacks of hereditary angioedema (HAE). Thus plasma kallikrein inhibition can be a useful treatment for HAE, and there is strong interest in the development of plasma kallikrein inhibitors as a therapy for HAE.

Hyperglycemic and diabetic individuals have an elevated risk of hemorrhage during thrombolytic therapy. In rodent models of intracerebral hemorrhage (ICH), it has been shown that KLKB1 inhibition or knockout reduces this effect. While the mechanism is not fully understood, this evidence suggests that plasma kallikrein inhibitors can be useful in the treatment of cerebral hemorrhage (Feener, E. P. Curr Diab Rep 2010, 10, 270).

Plasma kallikrein and Factor XIIa inhibitors have been shown to be neuroprotective in animal models of acute ischemic stroke and traumatic brain injury, reducing edema formation, inflammation, and thrombosis (Albert-WeiBenberger C, Sirén A L, Kleinschnitz C. Prog Neurobiol. 2013, 101-102, 65-82.). Thus, evidence suggests that plasma kallikrein inhibitors can be useful in the treatment of acute ischemic stroke and traumatic brain injury.

Plasma kallikrein can also cleave glucagon-like peptide 1 (GLP-1) and neuropeptide Y (NPY), both substrates for dipeptidyl peptidase-4 (DPP-4), a validated diabetes drug target. In the case of GLP-1, cleavage by KLKB1 reduces both its potency as well as plasma stability. In the case of NPY, cleavage by KLKB1 reduces its affinity to the Y2 and Y5 receptors. Thus, evidence suggests that plasma kallikrein inhibitors can be useful in the modulation of energy homeostasis and in the treatment of diabetes. (Feener, E. P. Curr Diab Rep 2010, 10, Feener, E. P. et al., Biol. Chem. 2013, 394, 319).

The Kallikrein-kinin system is involved in the regulation of vascular endothelial growth factor (VEGF), endothelial NO synthase, and fibroblast growth factor 2, all of which are involved in angiogenesis (Bader M. 2009, Arteriosclerosis, Thrombosis, and Vascular Biology, 29: 617). Tissue kallikrein (KLK1) has been linked to blood vessel growth (Miura S., 2003, Hypertension, 41, 1118). Therapies that moderate angiogenesis have been proposed for the treatment of both diabetic macular edema (DME) and age-related macular degeneration (AMD) (Syed, B. A.; Evans, J. B.; Bielory, L., 2012, Nature Reviews Drug Discovery, 11, 827). Without further wishing to be bound by any theory, it is therefore reasonable to conclude that KLK1 inhibitors can be useful in the treatment of diabetic retinopathy, DME, and AMD.

Studies have shown that inflammation plays an important role in the origin and development of AMD, and treatment often includes anti-inflammatories such as corticosteroid (Telander, D., 2011, Seminars in Ophthalmology, 26(3), 192). The connection between the kallikrein-kinin system and inflammation is also well established (Duchene, 2011, “Kallikrein-kinin kystem in inflammatory diseases”. Kinins. De Gruyter. 261). Without further wishing to be bound by any theory, it is reasonable to conclude that the anti-inflammatory nature of kallikrein (e.g. KLK1 and KLKB1) inhibitors can be useful in the treatment of AMD.

PF-04886847 is an inhibitor of plasma kallikrein and has shown to be effective at reducing 6-keto-PGF_{1α}, plasma levels in lipopolysaccharide (LPS) treated rats (Kolte, D et al. Cardiovascular & Hematological Agents in Medicinal Chemistry, 2012, 10, 154-166). Without further wishing to be bound by any theory, it is reasonable to believe that plasma kallikrein inhibitors can be useful in the treatment of hypotensive shock during sepsis.

Daiichi Seiyaku Co Ltd received approval in Japan to market cetraxate for gastritis and peptic ulcers. Cetraxate is reported as a plasma kallikrein inhibitor (WIPO Patent Application WO/2006/108643). Without further wishing to be bound by any theory, it is reasonable to believe that plasma kallikrein inhibition in general can be useful in the treatment of gastritis and peptic ulcers.

Fibrosis.

Kallikreins are a subgroup of serine proteases, divided into plasma kallikrein (KLKB1) and tissue kallikreins. KLKB1 liberates kinins (bradykinin and kallidin) from the kininogens, peptides responsible for the regulation of blood pressure and activation of inflammation. In the Contact Activation Pathway of the coagulation cascade, KLKB1 assists in the conversion of factor XII to factor XIIa (Keel, M.; Trentz, O. Injury 2005, 36, 691-709). Factor XIIa converts FXI into FXIa, which in turn activates FIX, which with its co-factor FVIIIa forms the tenase complex, which finally activates FX to FXa. In the fibrinolysis part of the coagulation cascade, KLKB1 serves to convert plasminogen to plasmin. Thus, it has been proposed that KLKB1 inhibitors can be useful in the treatment of thrombotic and fibrinolytic diseases and disease conditions (U.S. Pat. No. 7,625,944; Bird et al. Thrombosis and Hemostasis 2012, 107, 1141).

Several studies have shown the utility of anticoagulant therapy in fibrotic disorders. For example, in a rat model of CCl₄-induced chronic liver injury, the DTI SSR182289 decreased liver fibrogenesis significantly after 7 weeks of administration. Similar observations were made in other studies using the LMWHs nadroparin, tinzaparin, enoxaparin, and dalteparin sodium. See e.g., Duplantier, J. G., et al., 2004, Gut, 53:1682-1687; Abdel-Salam, O. M., et al., 2005, Pharmacol Res, 51:59-67; Assy, N., et al., 2007, Dig Dis Sci, 52:1187-1193; Abe, W., et al., 2007, J Hepatol, 46:286-294. Thus a thrombin inhibitor as an anticoagulant can be useful in the treatment of fibrinolytic diseases.

In another example, the DTI melagatran greatly reduced ischemia reperfusion injury in a kidney transplant model in the large white pig. This led to a drastically improved kidney graft survival at 3 months. See e.g., Favreau, F., et al., 2010, Am J Transplant, 10:30-39.

Recent studies have shown that in a bleomycin-induced mouse model of pulmonary fibrosis, dabigatran etexilate treatment reduced important profibrotic events in lung fibroblasts, including the production of collagen and connective tissue growth factor. See e.g., Silver, R. M., et al., 2010, Am. J. Respir. Crit. Care Med., 181:A6780; Bogatkevich, G. S., et al., 2009, Arthritis Rheum, 60:3455-3464.

The above experimental evidence points to a close relationship between thrombin and fibrosis and suggests novel therapeutic opportunities for fibrosis using thrombin inhibitors. See e.g., Calvaruso, V., et al., 2008, Gut, 57:1722-1727; Chambers, R. C., 2008, Br J Pharmacol, 153 Suppl 1:S367-378; Chambers, R. C. & Laurent, G. J., 2002, Biochem Soc Trans, 30:194-200; Howell, D. C., et al., 2001, Am J Pathol, 159:1383-1395.

Inflammation.

Kallikrein has long been implicated in inflammation (Clements, J. A. The Molecular Biology of the Kallikreins and Their Roles in Inflammation, Academic Press: San Diego, Calif., 1997; Vol. 5). There is experimental evidence that KLKB1 is associated with sepsis and inflammatory arthritis (Colman, R. W., 1998, Clinical Reviews in Allergy and Immunology, 16: 365). Thus a KLKB1 inhibitor can be useful in the treatment of inflammatory conditions associated with the kallikrein-kinin system, such as systemic inflammatory response syndrome, sepsis, rheumatoid arthritis, and inflammatory bowel disease.

Without further wishing to be bound by any theory, it is reasonable to believe that kallikrein inhibition in general can be useful for the treatment of kallikrein-related diseases or disorders and/or diseases or disorders. Such conditions include, for example, thrombotic diseases, fibrinolytic diseases, fibrotic disorders, cancer, inflammatory conditions, dermatological conditions, or the like.

Accordingly, in a further aspect, there is provided a method for treating a disease or disorder in a subject in need thereof. The method includes administering a compound of any of Formulae (Ia), (Ib), (II), (III), (IV), or (V) as disclosed herein, a compound as set forth in Table A, Table B, or Table C, or a pharmaceutically acceptable salt, ester, solvate, or prodrug thereof, or pharmaceutical composition thereof, to a subject in need thereof in an amount effective to treat the disease or disorder. The terms “therapeutically effective amount,” “amount effective to treat,” “amount effective to prevent” and the like refer to that amount of drug or pharmaceutical agent (e.g., compound or pharmaceutical composition disclosed herein) that will elicit the biological or medical response of a tissue, system, animal, or human that is being sought by a researcher, veterinarian, medical doctor or other clinician.

Compounds useful for methods disclosed herein include the compounds set forth for Formulae (Ia), (Ib), (II), (III), (IV), or (V) and for the compounds set forth in Table A, Table B, or Table C above.

In some embodiments of the methods described herein, the disease or disorder to be treated can include one or more thrombotic diseases or disorders and/or can involve a blood clot thrombus or the potential formation of a blood clot thrombus. In some embodiments, the thrombotic disease or disorder can be acute coronary syndrome, thromboembolism, and/or thrombosis. In some embodiments, the thromboembolism can be venous thromboembolism, arterial thromboembolism, and/or cardiogenic thromboembolism. In some embodiments, the venous thromboembolism can include deep vein thrombosis and/or pulmonary embolism. In some embodiments, the deep vein thrombosis and/or pulmonary embolism can occur following a medical procedure. In some embodiments, the thrombotic disease or disorder can involve dysfunctional coagulation or disseminated intravascular coagulation. In some embodiments, the subject with dysfunctional coagulation can be undergoing percutaneous coronary intervention (PCI). In some embodiments, the thrombotic disease or disorder can involve a blood clot thrombus or the potential formation of a blood clot thrombus and further can involve stroke and/or one or more transient ischemic attacks (TIA). In some embodiments, the thrombotic disease or disorder involving a blood clot thrombus or the potential formation of a blood clot thrombus can further involve stroke, wherein the subject can have non-valvular atrial fibrillation. In some embodiments, the thrombotic disease or disorder can involve a blood clot thrombus or the potential formation of a blood clot thrombus and further can involve pulmonary hypertension. In some embodiments, the pulmonary hypertension can be caused by one or more left heart disorder and/or chronic thromboembolic disease. In some embodiments, the pulmonary hypertension can be associated with one or more lung disease, including pulmonary fibrosis (idiopathic or otherwise), and/or hypoxia.

In some embodiments, the venous thromboembolism can be associated with formation of a thrombus within a vein associated with one or more acquired or inherited risk factors and/or embolism of peripheral veins caused by a detached thrombus. In some embodiments, the one or more risk factors can include a previous venous thromboembolism. In some embodiments, the cardiogenic thromboembolism can be due to formation of a thrombus in the heart associated with cardiac arrhythmia, heart valve defect, prosthetic heart valves or heart disease, and/or embolism of peripheral arteries caused by a detached thrombus. In some embodiments, the detached thrombus can be in the brain (ischemic stroke). In some embodiments, the detached thrombus can cause a transient ischemic attack (TIA). In some embodiments, the cardiogenic thromboembolism can be due to non-valvular atrial fibrillation. In some embodiments, the thrombosis can be arterial thrombosis. In some embodiments, the arterial thrombosis can be due to one or more underlying atherosclerotic processes in the arteries. In some embodiments, the one or more underlying atherosclerotic processes in the arteries can obstruct or occlude an artery, cause myocardial ischemia (angina pectoris, acute coronary syndrome), cause myocardial infarction, obstruct or occlude a peripheral artery (ischemic peripheral artery disease), and/or obstruct or occlude the artery after a procedure on a blood vessel (reocclusion or restenosis after transluminal coronary angioplasty, reocclusion or restenosis after percutaneous transluminal angioplasty of peripheral arteries).

In some embodiments, the disease or disorder can include fibrosis, Alzheimer's Disease, multiple sclerosis, pain, cancer, inflammation, and/or Type I diabetes mellitus. In some embodiments, the disease or disorder can involve recurrent cardiac events after myocardial infarction.

In some embodiments, the treatment or prevention can include an adjunct therapy. In some embodiments, the subject can have myocardial infarction, and the adjunct therapy can be in conjunction with thrombolytic therapy. In some embodiments, the subject can have unstable angina pectoris, thrombosis, and/or heparin-induced thrombocytopenia, and the adjunct therapy can be in combination with antiplatelet therapy. In some embodiments, the subject can have non-valvular atrial fibrillation, and the adjunct therapy can be in conjunction with one or more other therapies.

In some embodiments of the methods described herein, the disease or disorder can be a kallikrein-related disorder. In some embodiments, the kallikrein-related disorder can be a thrombotic disease, a fibrinolytic disease, a fibrotic disorder, a type of cancer, an inflammatory condition, or a dermatological condition.

In some embodiments, the kallikrein-related disorder can be an ophthalmic disease. In some embodiments, the compound or pharmaceutical composition can be administered in the form of an ophthalmic composition applied topically to the eye. In some embodiments, the ophthalmic composition can be in the form of eye drops. In some embodiments, the compound or pharmaceutical composition can be administered in the form of an ophthalmic composition via intravitreal injection. In some embodiments, the ophthalmic disease can be diabetic macular edema, hereditary angioedema, age-related macular degeneration, or diabetic retinopathy.

In some embodiments wherein the disease or disorder can be a type of cancer, said type of cancer can be cervical-, testicular-, or non-small-cell lung adenocarcinoma. In some embodiments, the cancer can be limited small cell lung cancer. In some embodiments, the cancer can be a glioma. In some embodiments, the cancer can be malignant breast cancer. In some embodiments, the cancer can be a micrometastasis. In some embodiments, the micrometastasis can be of the blood or liver. In some embodiments, the cancer can be a lung metastasis. In some embodiments, the cancer can be prostatic cancer.

In some embodiments wherein the disease or disorder can be an inflammatory condition, said inflammatory condition can be sepsis, inflammatory bowel disease, systemic inflammatory response syndrome, inflammatory arthritis, or rheumatoid arthritis.

In some embodiments wherein the disease or disorder can be a dermatological condition, said dermatological condition can be atopic dermatitis, psoriasis, or Netherton Syndrome.

In another aspect, there is provided a method for preventing a disease or disorder in a subject. The method includes administering a compound of any of Formulae (Ia), (Ib), (II), (III), (IV), or (V) as disclosed herein, compound as set forth in any of Table A, Table B, or Table C herein, pharmaceutically acceptable salt, ester, solvate, or prodrug thereof, or pharmaceutical composition thereof, to a subject in need thereof in an amount effective to prevent the disease or disorder.

### V. Assays

Compounds described herein can be assayed, by a variety of methods known in the art and described herein, for inhibition of biological activity, e.g., protease activity, of a variety of proteins, e.g., thrombin and KLKB1.

The thrombin activity reported herein (e.g., Table A) was obtained as follows. Human thrombin was obtained from Haematologic Technologies Inc. The chromogenic substrate S-2238 was obtained from DiaPharma. Thrombin was assayed in buffer containing 0.05 M Tris (pH 7.4), 0.015 M NaCl and 0.01% PEG-8000. The final concentration of enzyme used was 3 nM thrombin. The final concentration of substrate used was 125 μM S-2238 for thrombin. All assays were performed in 96-well microtiter plates at room temperature (RT). The enzyme and inhibitor were pre-incubated for 10 minutes then substrate was added and read at 405 nm in a SpectraMax Plus Spectrophotometer (Molecular Devices). Inhibitor IC₅₀ values were determined by adding test compound as ten point, three-fold serial dilutions in buffer solution, as known in the art. The plate was read at 10 minutes after substrate addition. The IC₅₀ was calculated by plotting the percent (%) inhibition against compound concentration and fitting the data to a constrained four parameter sigmoidal curve, as known in the art.

The KLKB1 kallikrein activity reported herein (e.g., Table B) was obtained as follows. Human KLKB1 protein was obtained from Enzyme Research Labs. The chromogenic substrate S-2302 was obtained from DiaPharma. KLKB1 was assayed in buffer containing 0.05 M Tris (pH 7.4), 0.01 M NaCl and 0.2% w/v PEG-8000. The final concentration of enzyme used was 3 nM KLKB1. The final concentration of substrate used was 250 μM S-2302 for KLKB1. All assays were performed in 96-well microtiter plates at room temperature (RT). The enzyme and inhibitor were pre-incubated for 10 minutes then substrate was added and read at 405 nm in a SpectraMax Plus Spectrophotometer (Molecular Devices). Inhibitor IC₅₀ values were determined by adding test compound as ten point, three-fold serial dilutions in buffer solution, as known in the art. The plate was read at 10 minutes after substrate addition. The IC₅₀ was calculated by plotting the percent (%) inhibition against compound concentration and fitting the data to a constrained four parameter sigmoidal curve, as known in the art.

Chymotrypsin activity can be obtained as follows. Human pancreas a-chymotrypsin is obtained from Sigma. The chromogenic substrate S-7388 is obtained from Sigma. The final concentration of substrate used is 250 uM for chymotrypsin. All assays are performed in 96-well microtiter plates at room temperature (RT). The enzyme and inhibitor are pre-incubated for 10 minutes then substrate is added and read at 405 nm in a SpectraMax Plus Spectrophotometer (Molecular Devices). Inhibitor IC₅₀ values are determined by adding test compound as ten point, three-fold serial dilutions in buffer solution, as known in the art. The plate is read at 5 minutes after substrate addition. The IC₅₀ is calculated by plotting the percent (%) inhibition against compound concentration and fitting the data to a constrained four parameter sigmoidal curve, as known in the art.

Factor XIa activity can be obtained as follows. Human Factor XIa is obtained from Enzyme Research. The chromogenic substrate S-2366 is obtained from DiaPharma. The final concentration of substrate used is 10 mM for Factor XIa. All assays re performed in 96-well microtiter plates at room temperature (RT). The enzyme and inhibitor are pre-incubated for 10 minutes then substrate is added and read at 405 nm in a SpectraMax Plus Spectrophotometer (Molecular Devices). Inhibitor IC₅₀ values are determined by adding test compound as ten point, three-fold serial dilutions in buffer solution, as known in the art. The plate is read at 10 minutes after substrate addition. The IC₅₀ is calculated by plotting the percent (%) inhibition against compound concentration and fitting the data to a constrained four parameter sigmoidal curve, as known in the art.

### VI. Pharmaceutical Compositions

In another aspect, there is provided a pharmaceutical composition comprising a compound disclosed herein and a pharmaceutically acceptable excipient. The compound is a compound of any of Formulae (Ia), (Ib), (II), (III), (IV), or (V) as disclosed herein, a compound as set forth in Table A or B herein, or pharmaceutically acceptable salt, ester, solvate, or prodrug thereof. In some embodiments, the compound is set forth in Table A or B herein.

The term “pharmaceutically acceptable salts” is meant to include salts of the active compounds that are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds disclosed herein contain both basic and acidic functionalities that allow the compounds to be converted into either base or acid addition salts.

Compounds disclosed herein can exist as salts, such as with pharmaceutically acceptable acids. Accordingly, the compounds contemplated herein include such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts can be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Pharmaceutically acceptable salts of the compounds above, where a basic or acidic group is present in the structure, are also included within the scope of compounds contemplated herein. When an acidic substituent is present, such as —NHSO₃H, —COOH and —P(O)(OH)₂, there can be formed the ammonium, sodium, potassium, calcium salt, and the like, for use as the dosage form. Basic groups, such as amino or basic heteroaryl radicals, or pyridyl and acidic salts, such as hydrochloride, hydrobromide, acetate, maleate, palmoate, methanesulfonate, p-toluenesulfonate, and the like, can be used as the dosage form.

Also, in the embodiments in which R—COOH is present, pharmaceutically acceptable esters can be employed, e. g., methyl, ethyl, tert-butyl, pivaloyloxymethyl, and the like, and those esters known in the art for modifying solubility or hydrolysis characteristics for use as sustained release or prodrug formulations.

A. Formulations

The compounds disclosed herein can be prepared and administered in a wide variety of ophthalmic, oral, parenteral, and topical dosage forms. The compounds described herein can be administered by eye drop. Also, compounds described herein can be administered by injection (e.g. intravenously, intramuscularly, intravitreally, intracutaneously, subcutaneously, intraduodenally, or intraperitoneally). As such, compounds described herein can also be administered by intravitreal injection. Also, the compounds described herein can be administered by inhalation, for example, intranasally. Additionally, the compounds disclosed herein can be administered transdermally. It is also envisioned that multiple routes of administration (e.g., intramuscular, oral, ocular) can be used to administer the compounds disclosed herein.

In some embodiments, the compounds disclosed herein can be prepared in liquid pharmaceutical compositions for ocular administration. The composition for ocular use can contain one or more agents selected from the group of buffering agents, solubilizing agents, coloring agents, viscosity enhancing agents, and preservation agents in order to produce pharmaceutically elegant and convenient preparations.

In some embodiments, the composition for ocular use can contain preservatives for protection against microbiological contamination, including but not limited to benzalkonium chloride and/or EDTA. Other possible preservatives include but are not limited to benzyl alcohol, methyl parabens, propyl parabens, and chlorobutanol. Preferably, a preservative, or combination of preservatives, will be employed to impart microbiological protection in addition to protection against oxidation of components.

In some embodiments, the compounds disclosed herein can be administered orally as tablets, aqueous or oily suspensions, lozenges, troches, powders, granules, emulsions, capsules, syrups or elixirs. The composition for oral use can contain one or more agents selected from the group of sweetening agents, flavoring agents, coloring agents and preserving agents in order to produce pharmaceutically elegant and palatable preparations. Accordingly, there are also provided pharmaceutical compositions comprising a pharmaceutically acceptable carrier or excipient and one or more compounds disclosed herein.

In some embodiments, tablets contain the acting ingredient in admixture with non-toxic pharmaceutically acceptable excipients that are suitable for the manufacture of tablets. These excipients can be, for example, (1) inert diluents, such as calcium carbonate, lactose, calcium phosphate, carboxymethylcellulose, or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets can be uncoated or coated by known techniques to delay disintegration and absorption in the gastrointestinal tract and thereby provide a sustained action over a longer period. For example, a time delay material such as glyceryl monostearate or glyceryl distearate can be employed.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substance that can also act as diluents, flavoring agents, binders, preservatives, tablet disintegrating agents, or an encapsulating material.

A compound disclosed herein, in the form of a free compound or a pharmaceutically-acceptable pro-drug, metabolite, analogue, derivative, solvate or salt, can be administered, for in vivo application, parenterally by injection or by gradual perfusion over time. Administration can be intravenously, intraperitoneally, intramuscularly, subcutaneously, intracavity, or transdermally. For in vitro studies the compounds can be added or dissolved in an appropriate biologically acceptable buffer and added to a cell or tissue.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds disclosed herein are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds disclosed herein can also be incorporated into liposomes or administered via transdermal pumps or patches. Pharmaceutical admixtures suitable for use in the pharmaceuticals compositions and methods disclosed herein include those described, for example, in PHARMACEUTICAL SCIENCES (17th Ed., Mack Pub. Co., Easton, Pa.) and WO 96/05309, the teachings of both of which are hereby incorporated by reference.

In some embodiments, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, growth factors and inert gases and the like.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations can contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation can be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds can have limited solubility in water and therefore can require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions can be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions disclosed herein can additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

By the present, there are provided methods for ameliorating wound healing and for mediating tissue repair (including but not limited to treatment of peripheral and coronary vascular disease). According to these methods, a subject having a wound or in need of tissue repair, is treated at the site of the wound or damaged tissue or treated systemically, with a compound disclosed herein in the form of a free compound or a pharmaceutically-acceptable prodrug, metabolite, analogue, derivative, solvate or salt.

Generally, the terms “treating”, “treatment” and the like are used herein to mean affecting a subject, tissue or cell to obtain a desired pharmacologic and/or physiologic effect. The effect can be prophylactic in terms of completely or partially preventing a disease or disorder or sign or symptom thereof, and/or can be therapeutic in terms of a partial or complete cure for a disorder and/or adverse effect attributable to it, e.g. pulmonary embolism following a medical procedure. “Treating” as used herein covers any treatment of, or prevention of a disease or disorder in a vertebrate, a mammal, particularly a human, and includes: (a) preventing the disease or disorder from occurring in a subject that can be predisposed to the disease or disorder, but has not yet been diagnosed as having it; (b) inhibiting the disease or disorder, i.e., arresting its development; or (c) relieving or ameliorating the disease or disorder, i.e., cause regression of the disease or disorder.

There are provided various pharmaceutical compositions useful for ameliorating certain diseases and disorders. The pharmaceutical compositions according to one embodiment are prepared by formulating a compound disclosed herein in the form of a free compound or a pharmaceutically-acceptable pro-drug, metabolite, analogue, derivative, solvate or salt, either alone or together with other pharmaceutical agents, suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers.

Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV, 14th ed. Washington: American Pharmaceutical Association (1975), the contents of which are hereby incorporated by reference. The pH and exact concentration of the various components of the pharmaceutical composition are adjusted according to routine skills in the art. See e.g., Goodman and Gilman (eds.), 1990, THE PHARMACOLOGICAL BASIS FOR THERAPEUTICS (7th ed.).

The pharmaceutical compositions are preferably prepared and administered in dose units. Solid dose units are tablets, capsules and suppositories. For treatment of a subject, depending on activity of the compound, manner of administration, nature and severity of the disease or disorder, age and body weight of the subject, different daily doses can be used.

Under certain circumstances, however, higher or lower daily doses can be appropriate. The administration of the daily dose can be carried out both by single administration in the form of an individual dose unit or else several smaller dose units and also by multiple administrations of subdivided doses at specific intervals.

The pharmaceutical compositions contemplated herein can be administered locally or systemically in a therapeutically effective dose. Amounts effective for this use will, of course, depend on the severity of the disease or disorder and the weight and general state of the subject. Typically, dosages used in vitro can provide useful guidance in the amounts useful for in situ administration of the pharmaceutical composition, and animal models can be used to determine effective dosages for treatment of particular disorders.

Various considerations are described, e. g., in Langer, 1990, Science, 249: 1527; Goodman and Gilman's (eds.), 1990, Id., each of which is herein incorporated by reference and for all purposes. Dosages for parenteral administration of active pharmaceutical agents can be converted into corresponding dosages for oral administration by multiplying parenteral dosages by appropriate conversion factors. As to general applications, the parenteral dosage in mg/mL times 1.8=the corresponding oral dosage in milligrams (“mg”). As to oncology applications, the parenteral dosage in mg/mL times 1.6=the corresponding oral dosage in mg. An average adult weighs about 70 kg. See e.g., Miller-Keane, 1992, ENCYCLOPEDIA & DICTIONARY OF MEDICINE, NURSING & ALLIED HEALTH, 5th Ed., (W. B. Saunders Co.), pp. 1708 and 1651.

The method by which the compound disclosed herein can be administered for oral use would be, for example, in a hard gelatin capsule wherein the active ingredient is mixed with an inert solid diluent, or soft gelatin capsule, wherein the active ingredient is mixed with a co-solvent mixture, such as PEG 400 containing Tween-20. A compound disclosed herein can also be administered in the form of a sterile injectable aqueous or oleaginous solution or suspension. The compound can generally be administered intravenously or as an oral dose of 0.1 μg to 20 mg/kg given, for example, every 3-24 hours.

Formulations for oral use can be in the form of hard gelatin capsules wherein the active ingredient is mixed with an inert solid diluent, for example, calcium carbonate, calcium phosphate or kaolin. They can also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients can be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which can be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions can be in the form of a sterile injectable aqueous or oleagenous suspension. This suspension can be formulated according to known methods using those suitable dispersing or wetting agents and suspending agents that have been mentioned above. The sterile injectable preparation can also a sterile injectable solution or suspension in a non-toxic parenterally-acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

A compound disclosed herein can also be administered in the form of ophthalmic compositions applied topically to the eye, preferably in the form of eye drops. A compound disclosed herein can also be administered in the form of intravitreal injection.

A compound disclosed herein can also be administered in the form of suppositories for rectal administration of the drug. These compositions can be prepared by mixing the drug with a suitable non-irritating excipient that is solid at ordinary temperature but liquid at the rectal temperature and will therefore melt in the rectum to release the drug. Such materials include cocoa butter and polyethylene glycols.

The compounds disclosed herein as used in the methods disclosed herein can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

For topical use, creams, ointments, jellies, solutions or suspensions, etc., containing the compounds disclosed herein, are employed.

In addition, some of the compounds disclosed herein can form solvates with water or common organic solvents. Such solvates are encompassed within the scope of the methods contemplated herein.

B. Effective Dosages

Pharmaceutical compositions provided herein include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to inhibition of thrombin, and/or KLKB1); presence of other diseases or other health-related problems; kind of concurrent treatment; and complications from any disease or treatment regimen. Other therapeutic regimens or agents can be used in conjunction with the methods and compounds disclosed herein.

For any compound described herein, the therapeutically effective amount can be initially determined from a variety of techniques known in the art, e.g., biochemical characterization of inhibition of enzyme (thrombin or KLKB1), cell culture assays, and the like. Target concentrations will be those concentrations of active compound(s) that are capable of decreasing enzymatic activity as measured, for example, using the methods described.

Therapeutically effective amounts for use in humans can be determined from animal models. For example, a dose for humans can be formulated to achieve a concentration that has been found to be effective in animals. The dosage in humans can be adjusted by monitoring enzymatic inhibition and adjusting the dosage upwards or downwards, as described above.

Dosages can be varied depending upon the requirements of the patient and the compound being employed. The dose administered to a patient, in the context of the methods disclosed herein, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In some embodiments of a method disclosed herein, the dosage range is 0.001% to 10% w/v. In some embodiments, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

Accordingly, in some embodiments, dosage levels of the compounds disclosed herein as used in the present methods are of the order of e.g., about 0.1 mg to about 1 mg, about 1 mg to about 10 mg, about 0.5 mg to about 20 mg per kilogram body weight, an average adult weighing 70 kilograms, with a preferred dosage range between about 0.1 mg to about 20 mg per kilogram body weight per day (from about 7.0 mg to about 1.4 gm per patient per day). The amount of the compound disclosed herein that can be combined with the carrier materials to produce a single dosage will vary depending upon the host treated and the particular mode of administration. For example, a formulation intended for oral administration to humans can contain about 5 μg to 1 g of a compound disclosed herein with an appropriate and convenient amount of carrier material that can vary from about 5 to 95 percent of the total composition. Dosage unit forms will generally contain between from about 0.1 mg to 500 mg of a compound disclosed herein.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from in vitro assays, cell culture assays and/or animal studies can be used in formulating a range of dosages for use in humans. The dosage of such compounds preferably lies within a range of plasma concentrations that include the ED₅₀ with little or no toxicity. The dosage can vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual practitioner in view of the patient's condition and the particular method in which the compound is used. For in vitro formulations, the exact formulation and dosage can be chosen by the individual practitioner in view of the patient's condition and the particular method in which the compound is used.

### VII. Examples

The examples below are meant to illustrate certain embodiments of the invention and not to limit the scope of the invention. Abbreviations used herein have their conventional meaning in the art, unless indicated otherwise. Specific abbreviations include the following: Å=Ångstrom; Ac₂O=acetic anhydride; AcOH=acetic acid; aq=aqueous; Bt=benzotriazole; BOC=N-tert-butoxycarbonyl; br=broad; t-BuOH=tert-butanol; ° C.=degree Celsius; d=doublet; DABCO=1,4-diazabicyclo[2.2.2]octane; DCE=1,2-dichloroethane; DCM=dichloromethane; dd=doublet of doublets; DIEA=diethylisopropylamine; DMAP=4-dimethylaminopyridine; DMF=N,N-dimethylformamide; DMSO=dimethylsulfoxide; δ=chemical shift (given in ppm, unless otherwise indicated); EDCI=1-ethyl-3-(3-dimethylaminopropyl)carbodiimide; eq=equivalent; Et₂O=diethyl ether; Et₃N=triethylamine; EtOAc=ethyl acetate; EtOH=ethanol; g=gram; h (or hr)=hour; HOBt=hydroxybenzotriazole; HPLC=high performance liquid chromatography; Hz=Hertz; IC₅₀=inhibitory concentration at 50% inhibition; J=coupling constant (given in Hz, unless otherwise indicated); LC=liquid chromatography; LHMDS=lithium hexamethyldisilazide; m=multiplet; M=molar; [M+H]⁺=parent mass spectrum peak plus H⁺; MS=mass spectrum; ms=molecular sieves; MP=melting point; Me₂NH=dimethylamine; MeOH=methanol; mg=milligram; mL=milliliter; mM=millimolar; mmol=millimole; min=minute; μL=microliter; μM=micromolar; ng=nanogram; nM=nanomolar; NMR=nuclear magnetic resonance; ppm=parts per million; q=quartet; R_{f}=retention factor; RT=room temperature; s=singlet; t=triplet; TFA=trifluoroacetic acid; THF=tetrahydrofuran; TLC=thin layer chromatography.

General Scheme Ia.

A synthetic scheme useful for synthesis of compounds described herein is disclosed in General Scheme Ia following, wherein IIIa in General Scheme Ia is more generally described by Formula III, and the terms “V”, “W”, “X”, “Y”, “Z”, “R¹” and “R²” are as defined above.

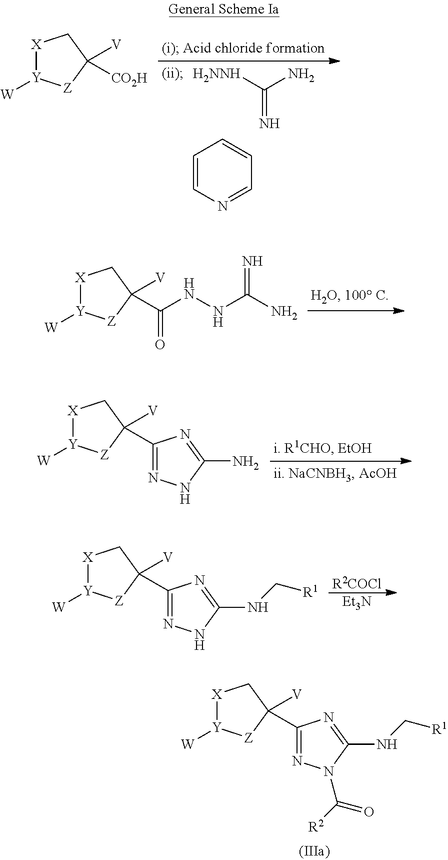

General Scheme Ib.

Equally, a synthetic scheme useful for the synthesis of compounds is disclosed in General Scheme Ib following, wherein IIIb in General Scheme Ib is more generally described by Formula III, and the terms “V”, “IV”, “X”, “Y”, “Z”, “R¹” and “R²” are as defined above.

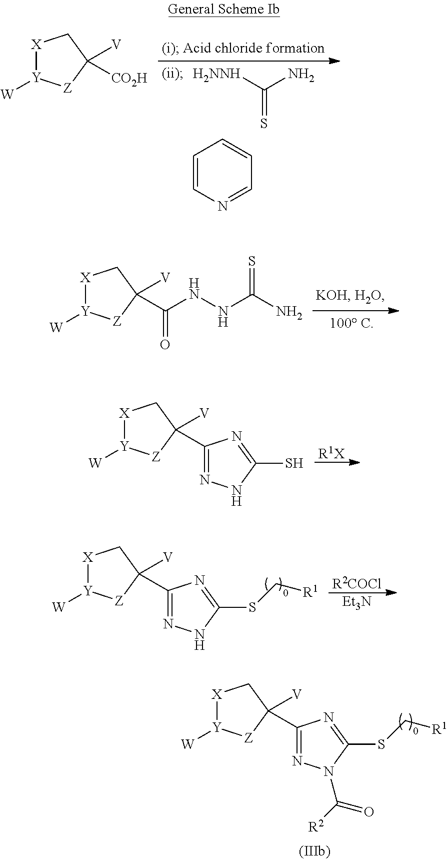

Specific synthetic routes can be designed to provide the compounds described herein, as well as the specific compounds listed on Table A, Table B, and Table C, via chemical reactions known to those skilled in the art. Exemplary methods to attach various groups are disclosed in U.S. Provisional Application No. 62/126,424, SUBSTITUTED PYRAZOLE COMPOUNDS AS SERINE PROTEASE INHIBITORS, filed on Feb. 27, 2015, and International Application No. PCT/US2016/20116, SUBSTITUTED PYRAZOLE COMPOUNDS AS SERINE PROTEASE INHIBITORS, filed on Feb. 29, 2016, which are both incorporated by reference herein in their entirety and for all purposes. Further, the contents of all references, patents, and published applications cited herein are hereby incorporated by reference in their entirety and for all purposes.

While the invention has been described in detail with reference to certain preferred embodiments thereof, it will be understood that modifications and variations are within the spirit and scope of that which is described and claimed.

What is claimed is:

1. A compound with the following structure: 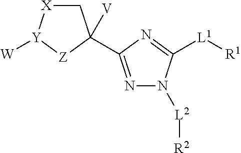
or pharmaceutically acceptable salt, ester, solvate, or prodrug thereof, wherein:
L¹ and L² are independently a bond, substituted or unsubstituted alkylene, substituted or unsubstituted heteroalkylene, —C(O)—, —S—, —SO—, —SO₂—, —O—, —NHSO₂—, —NHC(O)—, or —NR⁴—;
R¹ and R² are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted fused ring aryl, or substituted or unsubstituted heteroaryl;
R⁴ is hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted fused ring aryl, or substituted or unsubstituted heteroaryl;
V is hydrogen or substituted or unsubstituted alkyl;
W is absent, hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted heteroaryl, —C(O)R⁶, —C(O)OR⁶, —SR⁶, —SOR⁶, —SO₂R⁶, —SO₂NR⁶R⁷, —OR⁶, —NHSO₂R⁶, or —NR⁶R⁷, where R⁶ and R⁷ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, or substituted or unsubstituted heteroaryl, wherein R⁶ and R⁷ can be combined if both are present to form a substituted or unsubstituted alkylene, or substituted or unsubstituted heteroalkylene;
X is a bond, substituted or unsubstituted alkylene, —O—, or —NR⁸—;
Y is a bond, substituted or unsubstituted alkylene, —O—, or —N—, provided that when Y is —O—, W is absent; and
Z is a bond, —C(O)—, substituted or unsubstituted alkylene, —O—, or —NR⁹—;
wherein R⁸ and R⁹ are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted cycloalkenyl, substituted or unsubstituted heterocycloalkenyl, substituted or unsubstituted aryl, substituted or unsubstituted heteroaryl, —C(O)R⁶, —C(O)OR⁶, —C(O)NR⁶R⁷, —SR⁶, —SOR⁶, —SO₂R⁶, —SO₂NR⁶R⁷, —OR⁶, —NHSO₂R⁶, or —NR⁶R⁷, wherein R⁶ and R⁷ are as defined above; and
provided that either at least one of X is —O— or —NR⁸—, Y is —O— or —N—, or Z is —O— or —NR⁹—.

2. The compound of claim 1, wherein X is a bond or substituted or unsubstituted alkylene.

3. The compound of claim 2, wherein Z is a bond or substituted or unsubstituted alkylene.

4. The compound of claim 3, wherein Y is —N—, and W is hydrogen, substituted or unsubstituted alkyl, substituted or unsubstituted heteroalkyl, substituted or unsubstituted aryl, —C(O)R⁶, —C(O)OR⁶, —C(O)NR⁶R⁷′—SO₂R⁶, or SO₂NR⁶R⁷, wherein R⁶ and R⁷ are independently substituted or unsubstituted alkyl, substituted or unsubstituted cycloalkyl, or substituted or unsubstituted heterocycloalkyl, or R⁶ and R⁷ can be combined if both are present to form a substituted or unsubstituted alkylene, or substituted or unsubstituted heteroalkylene.

5. The compound of claim 4, wherein X is selected from the group consisting of substituted or unsubstituted methylene, substituted or unsubstituted ethylene, substituted or unsubstituted propylene, substituted or unsubstituted butylene, and substituted or unsubstituted pentylene, and Z is a bond.

6. The compound of claim 4, wherein X is a bond, and Z is selected from the group consisting of substituted or unsubstituted methylene, substituted or unsubstituted ethylene, substituted or unsubstituted propylene, substituted or unsubstituted butylene, and substituted or unsubstituted pentylene.

7. The compound of claim 4, wherein X is selected from the group consisting of substituted or unsubstituted methylene, substituted or unsubstituted ethylene, substituted or unsubstituted propylene, substituted or unsubstituted butylene, and substituted or unsubstituted pentylene, and wherein Z is selected from the group consisting of substituted or unsubstituted methylene, substituted or unsubstituted ethylene, substituted or unsubstituted propylene, substituted or unsubstituted butylene, and substituted or unsubstituted pentylene.

8. The compound of claim 7, wherein X and Z are both branched alkylene and X and Z are covalently attached.

9. The compound of any of claims 6-8, wherein Z is selected from the group consisting of substituted methylene, substituted ethylene, substituted propylene, substituted butylene, and substituted pentylene, having one or more substituent groups selected from the group consisting of —OH, —NH₂, —SH, —CN, —CF₃, —NO₂, oxo, halogen, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

10. The compound of claim 4, wherein X is selected from the group consisting of substituted or unsubstituted methylene, substituted or unsubstituted ethylene, substituted or unsubstituted propylene, substituted or unsubstituted butylene, and substituted or unsubstituted pentylene, and wherein Z is —C(O)—.

11. The compound of any of claims 4-10, wherein W is hydrogen.

12. The compound of any of claims 4-10, wherein W is selected from the group consisting of substituted or unsubstituted alkyl, substituted or unsubstituted heteroalkyl, substituted or unsubstituted alkenyl, substituted or unsubstituted heteroalkenyl, substituted or unsubstituted cycloalkyl, substituted or unsubstituted heterocycloalkyl, —C(O)R⁶, —C(O)OR⁶, —C(O)NR⁶R⁷′—SR⁶, —SOR⁶, —SO₂R⁶, and —SO₂NR⁶.

13. The compound of claim 12, wherein W is substituted alkyl, substituted heteroalkyl, substituted alkenyl, substituted heteroalkenyl, substituted cycloalkyl, or substituted heterocycloalkyl, having one or more substituent groups selected from the group consisting of —OH, —NH₂, —SH, —CN, —CF₃, —NO₂, oxo, halogen, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

14. The compound of claim 12, wherein W is —COR⁶, —C(O)OR⁶, —C(O)NR⁶R⁷, —SO₂R⁶ or —SO₂NR⁶R⁷, and wherein R⁶ and R⁷ are selected from the group consisting of substituted or unsubstituted alkyl, substituted or unsubstituted heteroalkyl, substituted or unsubstituted aryl, and substituted or unsubstituted heteroaryl, or R⁶ and R⁷ combine to form a substituted or unsubstituted alkylene.

15. The compound of claim 1, wherein W is absent, X is —NR^{B}—, Y is a bond or substituted or unsubstituted alkylene, and Z is —NR⁹—.

16. The compound of claim 15, wherein Y is selected from the group consisting of substituted or unsubstituted methylene, substituted or unsubstituted ethylene, substituted or unsubstituted propylene, substituted or unsubstituted butylene, and substituted or unsubstituted pentylene.

17. The compound of claim 16, wherein R⁸ is selected from the group consisting of substituted or unsubstituted alkyl, substituted or unsubstituted heteroalkyl, substituted or unsubstituted alkenyl, substituted or unsubstituted heteroalkenyl, —COR⁶, —C(O)OR⁶, —C(O)NR⁶R⁷, —SR⁶, —SOR⁶, —SO₂R⁶, and —SO₂NR⁶R⁷, and wherein R⁹ is selected from the group consisting of substituted or unsubstituted alkyl, substituted or unsubstituted heteroalkyl, substituted or unsubstituted alkenyl, substituted or unsubstituted heteroalkenyl, —COR⁶, —C(O)OR⁶, —C(O)NR⁶R⁷, —SR⁶, —SOR⁶, —SO₂R⁶, and —SO₂NR⁶R⁷.

18. The compound of claim 3, wherein Y is —O—, and W is absent.

19. The compound of claim 18, wherein X is selected from the group consisting of substituted or unsubstituted methylene, substituted or unsubstituted ethylene, substituted or unsubstituted propylene, substituted or unsubstituted butylene, and substituted or unsubstituted pentylene, and Z is a bond.

20. The compound of claim 18, wherein X is a bond, and Z is selected from the group consisting of substituted or unsubstituted methylene, substituted or unsubstituted ethylene, substituted or unsubstituted propylene, substituted or unsubstituted butylene, and substituted or unsubstituted pentylene.

21. The compound of claim 18, wherein X is selected from the group consisting of substituted or unsubstituted methylene, substituted or unsubstituted ethylene, substituted or unsubstituted propylene, substituted or unsubstituted butylene, and substituted or unsubstituted pentylene, and wherein Z is selected from the group consisting of substituted or unsubstituted methylene, substituted or unsubstituted ethylene, substituted or unsubstituted propylene, substituted or unsubstituted butylene, and substituted or unsubstituted pentylene.

22. The compound of any of claims 1-21, wherein V is hydrogen or substituted or unsubstituted methyl.

23. The compound according to any of claims 1-22, wherein L¹ is —S—, —O—, —NR⁴—, substituted or unsubstituted alkylene, or substituted or unsubstituted heteroalkylene; R¹ is substituted or unsubstituted alkyl, substituted or unsubstituted aryl, substituted or unsubstituted fused ring aryl, substituted or unsubstituted heteroaryl, or substituted or unsubstituted heterocycloalkyl; and R⁴ is hydrogen, substituted or unsubstituted alkyl, or substituted or unsubstituted heterocycloalkyl.

24. The compound according to claim 23, wherein L¹ is —NR⁴— or substituted or unsubstituted heteroalkyl, and R¹ is substituted or unsubstituted alkyl or substituted or unsubstituted heteroaryl.

25. The compound according to claim 24, wherein L¹ is —NR⁴—, and R¹ is substituted alkyl having one or more substituent groups selected from the group consisting of substituted or unsubstituted heteroaryl and substituted or unsubstituted heterocycloalkyl.

26. The compound according to claim 25, wherein R¹ is substituted alkyl substituted by chloro-substituted thiophenyl.

27. The compound according to claim 24, wherein L¹ is substituted or unsubstituted heteroalkyl, and R¹ is substituted or unsubstituted heteroaryl.

28. The compound according to any of claims 1-27, wherein L² is bond, substituted or unsubstituted alkylene, —C(O)—, or —SO₂—, and R² is hydrogen, substituted or unsubstituted alkyl, substituted or unsubstituted heteroalkyl, substituted or unsubstituted cycloalkyl, substituted or unsubstituted aryl, substituted or unsubstituted fused ring aryl, substituted or unsubstituted heteroaryl, or substituted or unsubstituted heterocycloalkyl.

29. The compound according to claim 28, wherein L² is bond, and R² is hydrogen.

30. The compound according to claim 28, wherein L² is —C(O)—, and R² is substituted or unsubstituted alkyl, substituted or unsubstituted heteroalkyl, substituted or unsubstituted cycloalkyl, substituted or unsubstituted aryl, substituted or unsubstituted fused ring aryl, substituted or unsubstituted heteroaryl, or substituted or unsubstituted heterocycloalkyl.

31. The compound according to any of claims 1 to 31 as set forth in Table A, Table B, or Table C.

32. A pharmaceutical composition comprising a compound according to any of claims 1 to 31, or a compound as set forth in Table A, Table B, or Table C, and a pharmaceutically acceptable excipient.

33. A method for treating and/or preventing a disease or disorder in a subject, comprising administering a compound according to any of claims 1 to 31 or a pharmaceutical composition according to claim 32, to a subject in need thereof in an amount effective to treat or prevent said disease or disorder.

34. The method according to claim 33, wherein said disease or disorder is a thrombotic disease or disorder and/or involves a blood clot thrombus or the potential formation of a blood clot thrombus.

35. The method according to claim 34, wherein said thrombotic disease or disorder comprises acute coronary syndrome, thromboembolism, and/or thrombosis.

36. The method according to claim 35, wherein the thromboembolism comprises venous thromboembolism, arterial thromboembolism, and/or cardiogenic thromboembolism.

37. The method according to claim 36, wherein the venous thromboembolism comprises deep vein thrombosis and/or pulmonary embolism.

38. The method according to claim 37, wherein the deep vein thrombosis and/or pulmonary embolism occurs following a medical procedure.

39. The method according to claim 34, wherein said thrombotic disease or disorder involves dysfunctional coagulation or disseminated intravascular coagulation.

40. The method according to claim 39, wherein the subject is undergoing percutaneous coronary intervention (PCI).

41. The method according to claim 34, wherein said thrombotic disease or disorder involves a blood clot thrombus or the potential formation of a blood clot thrombus and further involves stroke and/or one or more transient ischemic attacks (TIA).

42. The method according to claim 41, wherein said thrombotic disease or disorder involving a blood clot thrombus or the potential formation of a blood clot thrombus further involves stroke and wherein the subject has non-valvular atrial fibrillation.

43. The method according to claim 34, wherein said thrombotic disease or disorder involves a blood clot thrombus or the potential formation of a blood clot thrombus and further involves pulmonary hypertension.

44. The method according to claim 43, wherein the pulmonary hypertension is caused by one or more left heart disorder and/or chronic thromboembolic disease.

45. The method according to claim 43, wherein the pulmonary hypertension is associated with one or more lung disease, including pulmonary fibrosis (idiopathic or otherwise), and/or hypoxia.

46. The method according to claim 33, wherein said disease or disorder comprises fibrosis, Alzheimer's Disease, multiple sclerosis, pain, cancer, inflammation, and/or Type I diabetes mellitus.

47. The method according to claim 33, wherein the disease or disorder involves recurrent cardiac events after myocardial infarction.

48. The method according to claim 36, wherein the venous thromboembolism is associated with formation of a thrombus within a vein associated with one or more acquired or inherited risk factors and/or embolism of peripheral veins caused by a detached thrombus.

49. The method according to claim 48, wherein the one or more risk factors comprise a previous venous thromboembolism.

50. The method according to claim 36, wherein the cardiogenic thromboembolism is due to formation of a thrombus in the heart associated with cardiac arrhythmia, heart valve defect, prosthetic heart valves or heart disease, and/or embolism of peripheral arteries caused by a detached thrombus.

51. The method according to claim 50, wherein the detached thrombus is in the brain (ischemic stroke).

52. The method according to claim 51, wherein the detached thrombus causes a transient ischemic attack (TIA).

53. The method according to claim 50, wherein the cardiogenic thromboembolism is due to non-valvular atrial fibrillation.

54. The method according to claim 35, wherein the thrombosis is arterial thrombosis.

55. The method according to claim 54, wherein the arterial thrombosis is due to one or more underlying atherosclerotic processes in the arteries.

56. The method according to claim 55, wherein the one or more underlying atherosclerotic processes in the arteries obstruct or occlude an artery, cause myocardial ischemia (angina pectoris, acute coronary syndrome), cause myocardial infarction, obstruct or occlude a peripheral artery (ischemic peripheral artery disease), and/or obstruct or occlude the artery after a procedure on a blood vessel (reocclusion or restenosis after transluminal coronary angioplasty, reocclusion or restenosis after percutaneous transluminal angioplasty of peripheral arteries).

57. The method according to claim 33, wherein the treatment or prevention comprises an adjunct therapy.

58. The method according to claim 57, wherein the subject has myocardial infarction, and the adjunct therapy is in conjunction with thrombolytic therapy.

59. The method according to claim 57, wherein the subject has unstable angina pectoris, thrombosis, and/or heparin-induced thrombocytopenia, and the adjunct therapy is in combination with antiplatelet therapy.

60. The method according to claim 57, wherein the subject has non-valvular atrial fibrillation, and the adjunct therapy is in conjunction with one or more other therapies.

61. The method according to claim 33, wherein said disease or disorder is a kallikrein-related disorder.

62. The method according to claim 61, wherein said kallikrein-related disorder is a thrombotic disease, a fibrinolytic disease, a fibrotic disorder, a type of cancer, an inflammatory condition, or a dermatological condition.

63. The method according to claim 61, wherein said kallikrein-related disorder is an ophthalmic disease.

64. The method according to claim 63, wherein said compound or pharmaceutical composition is administered in the form of an ophthalmic composition applied topically to the eye.

65. The method according to claim 64, wherein the ophthalmic composition is in the form of eye drops.

66. The method according to claim 63, wherein said compound or pharmaceutical composition is administered in the form of an ophthalmic composition via intravitreal injection.

67. The method according to claim 63, wherein said ophthalmic disease is diabetic macular edema, hereditary angioedema, age-related macular degeneration, or diabetic retinopathy.

68. The method according to claim 62, wherein said type of cancer is selected from the group consisting of cervical-, testicular-, or non-small-cell lung adenocarcinoma, limited small cell lung cancer, glioma, malignant breast cancer, micrometastasis (e.g. micrometastasis of blood or liver), lung metastasis, and prostatic cancer.

69. The method according to claim 62, wherein said inflammatory condition is sepsis, inflammatory bowel disease, inflammatory arthritis, systemic inflammatory response syndrome, or rheumatoid arthritis.

70. The method according to claim 62, wherein said dermatological condition is atopic dermatitis, psoriasis, or Netherton Syndrome.

71. The method according to any of claims 33-70, wherein said compound acts by inhibiting thrombin and/or kallikrein.

72. The method according to claim 71, wherein said compound acts by inhibiting tissue kallikrein and/or plasma kallikrein.

73. The method according to claim 33, wherein the amount of compound administered is a therapeutically effective dose sufficient to achieve an initial concentration of the compound or its active metabolite(s) in plasma within a range of 1-10 nM, 10-100 nM, 0.1-1 μM, 1-10 μM, 10-100 μM, 100-200 μM, 200-500 μM, or 500-1000 μM, or greater.

74. The method according to claim 73, wherein greater than 50% of the initial compound concentration persists in the plasma for 1 hour, 3 hours, or longer, after intravenous injection.

75. A compound according to any of claims 1 to 31 or a pharmaceutical composition according to claim 32 for use in a method according to any of claims 33-74.

76. The compound according to any of claims 1-31, wherein the compound has inhibitory activity against thrombin and/or plasma kallikrein within a range of 1-10 nM, 10-100 nM, 0.1-1 μM, 1-10 μM, 10-100 μM, 100-200 μM, 200-500 μM, or 500-1000 μM, or greater.
